(19) United States
(12) Patent Application Publication
(10) Pub. No.: US 2011/0092380 A1
(43) Pub. Date: Apr. 21, 2011

(54) IMPROVED MOLECULAR-BIOLOGICAL PROCESSING EQUIPMENT

(75) Inventors: Peer Stahler, Mannheim (DE); Markus Beier, Weinheim (DE); Cord Stahler, Weinheim (DE); Daniel Summerer, Weinheim (DE); Mark Matzas, Heidelberg (DE); Sonja Vorwerk, Dossenheim (DE)

(73) Assignee: FEBIT HOLDING GMBH, Heidelberg (DE)

(21) Appl. No.: 12/521,765

(22) PCT Filed: Dec. 31, 2007
(86) PCT No.: PCT/EP2007/011478

§ 371 Date: Nov. 24, 2009

(30) Foreign Application Priority Data
Dec. 29, 2006 (DE) 10 2006 062 089.5
Apr. 20, 2007 (DE) 10 2007 018 833.3

(51) Int. Cl. C40B 30/04; C40B 60/14; C12Q 1/68; C12P 19/34; C40B 50/00; C40B 30/00; C40B 50/06; C40B 40/00; C40B 40/06

(52) U.S. Cl. 506/9; 506/37; 435/6; 435/91.2; 506/23; 506/7; 506/26; 506/13; 506/16

(57) ABSTRACT

The invention relates to improved molecular-biological processing equipment and an improved method of processing biological samples. The invention combines the provision of biologically functional molecules such as nucleic acids and peptides and of derivatives or analogs of these two classes of molecules in miniaturized flow cells with the sequential addition of reagents or fluids and serves for the processing of biological samples, such as proteins, nucleic acids, biogenic small molecules such as e.g. metabolites, viruses or cells, which for this purpose are introduced into the miniaturized flow cells. The invention further relates to methods and to the use of the molecular-biological processing equipment according to the invention for the detection and/or for the isolation of nucleic acids; for sequencing; for point mutation analysis; for the analysis of genomes and/or chromosomes; for the production of synthetic nucleic acids; for the production of arrays of primers, probes and/or antisense molecules; and other analytical and synthetic methods.

### 1 INTRODUCTION

For a comprehensive understanding of the molecular biology of humans and other living organisms it is necessary to know the number, nature and the interactions of all their genes and gene products with one another and with the environment. For this we need analytical techniques that provide information about the local concentration of genes and their encoded functional biomolecules (RNA, proteins) as well as other classes of substances that result from the activity of genes (e.g. products of enzymatic reactions such as certain metabolites) at a given point of time in an organism under defined conditions. The complete information for the manner of functioning of a living organism is encoded in its DNA. The latter in its turn codes for RNA, which can code for proteins. Owing to their stability and pairing properties, DNA and RNA can be investigated relatively easily, so existing analytical techniques are aimed in particular at these two classes of molecules. The focus then is on investigation of the DNA sequence of various organisms and various individuals of a species and their comparison with one another (e.g. genome sequencing, genotyping through analysis of sites of high genetic variability such as “single nucleotide polymorphisms”, evolutionary biology, taxonomy). Another important analytical technique is characterization of the nature and concentration of mRNA (expression profiling) in order to find information about the activity of genes under defined conditions. Such methods have led in recent years to an abundance of new knowledge.

Thus, according to the present state of research, the human genome and the genomes of some other complex living organisms such as the mouse and of a large number of small organisms, viruses, bacteria etc. are considered to be elucidated in terms of the sequence. However, elucidation of the function of our genome and of all other genomes is still right at the beginning. Sequencing has shown that humans possess far fewer genes than was assumed initially, and comparison of the sequenced organisms has shown that the number of genes and hence the number of proteins do not correlate with the complexity of an organism. Moreover, the differences in the genes are also minimal. Thus, the differences between the genes of two humans are in the per-thousand range and the difference between human and ape is just in the lower, single-digit percentage range—a very small genotypic difference compared with the obviously large difference in the phenotype.

In the March 2005 issue of “Spektrum der Wissenschaft” the author John S. Mattick from the University of Queensland, Australia, describes in his report “The unrecognized genome-program” an alternative or expanded model for the functioning of DNA. The current school of thought sees DNA just as the instructions for constructing proteins, and by definition each gene codes for one protein. This is largely true for primitive organisms, but for complex organisms, and above all humans, sequencing has shown that only 1.5% of human DNA codes for proteins and these segments, the so-called exons, are not arranged continuously on the DNA. Between the exons there are the introns, which together with the exons make up about 60% of human DNA. Therefore a human gene is now defined as a region of the DNA between a start codon and a stop codon, within which there are several exons, which code for one or more similar proteins. It therefore follows that the human genes each represent, as it were, a modular construction kit for different but related proteins. Therefore a human being, with about 25 000 genes, can produce about 100 000 proteins.

The introns and the remaining, noncoding DNA are regarded according to this model as Junk DNA or genetic waste. Altogether, then, 98.5% of the human genome would have no importance or no substantial importance, and the large amount is explained by the long duration of evolution. It is precisely here that Mattick assumes and postulates that the introns at least, and possibly all of the DNA not coding for proteins, contains the information for utilization of the genes. According to this, introns code for an enormous number of RNAs of various lengths. In fact, since then a large number of regulatory RNAs has been discovered (e.g. microRNAs), which are encoded outside of the exons.

Knowledge is also now being obtained on a large scale about the complex regulatory networks of genes at the protein level. In particular, mechanisms are at the forefront that are based on various post-translational protein modifications, which are not detectable at the nucleic acid level.

All these findings open up a large number of new research areas and require new flexible analytical techniques, which take into account the high throughput and the rapid change in knowledge in these areas. It may in particular also be of great utility to develop methods that enable a large number of biomolecules to be produced in parallel and to be investigated for particular properties in high throughput.

### 2 PRIOR ART

Methods for the investigation of biological samples are often based on chemical or biochemical manipulations of the sample coupled with physical methods of analysis. The following methods, for example, are known by a person skilled in the art:

### 2.1 Capillary Electrophoresis (CE)

The predominant method of sequence analysis is capillary electrophoresis (CE). Electric current is applied to capillaries filled with special gel, causing movement of charged molecules. Smaller molecules pass more quickly than large ones through the network of the gel. If a cocktail of molecules of different sizes is fed to a CE, these emerge from the capillary sorted according to their size. For the sequencing of charged DNA, this method is used in conjunction with an enzymatic assay. This sample preparation assay produces, starting from one end of the DNA to be sequenced, the respective transcript by primer extension. During this, at each copied nucleotide position a small proportion of nucleotide analogs is incorporated, which cause chain termination and in each case contain a nucleotide-specific fluorophor. Depending on which of the 4 bases stands at the end of the particular segment, the identity of a base at a position is analyzed based on the length and color of the resultant molecule. The colors are detected optically at the outlet from the capillary and the signals are processed by computer and assembled into a sequence.

This method is extensively automated. The largest machines from the market leader Applied Biosystems in the USA have up to 96 parallel capillaries with a reading rate of 650 bases per measurement and per capillary and therefore up to 1.5 million bases per machine per day (24 h). However, the maximum length that can be read at once per capillary is about 400 bases, which reduces the throughput per day to about 1 million bases per machine.

The method is mature and is widely established. However, the high costs mean that its use only makes economic sense in initial sequencing and for checking a moderate number of samples in re-sequencing and genotyping. To be able to sequence less important organisms or larger groups or even individual patients there would have to be a considerable cost reduction.

### 2.2 Polymerase Chain Reaction (PCR)

PCR and related methods use particularly temperature-stable enzymes, which were obtained from nature, for amplification of DNA or also RNA, comparable to the processes in a cell. This is necessary because in most cases the DNA/RNA in the sample to be measured is at such a low concentration that it cannot be detected by most methods of measurement. The PCR method is a “one-pot reaction”, in which the temperature is varied cyclically: from the denaturation step up to almost 100° C., addition steps at approx. 50° C. to 65° C. to enzymatic steps at approx. 72° C. Depending on the assay, this results in a linear or even exponential amplification of the sequence between two suitably selected primers, oligonucleotides with the complementary sequence to the region to which they attach, and which therefore make the enzyme reaction possible. By selecting the primer sequences it is possible to determine which region is multiplied, i.e. amplified. In addition to sample preparation for other detection techniques, e.g. DNA arrays, this is also used for detection directly. When using PCR as an analytical technique (direct detection) the product of an amplification is stained and detected. Just the presence of one or more successful amplifications in a particular starting material permits recognition. The method is widely established and recognized and, especially in calibrated form as quantitative PCR, provides reliable results. The PCR method does, however, have two important weak points. One is the relatively high costs for a primer pair. This is less of a disadvantage when one primer set is used for several reactions. A rule of thumb is 100 reactions with one set. The second disadvantage, especially with analyses that are less well understood or complex, is that no information at all is obtained concerning the sequence between the two primers. Therefore any other sequence can also be amplified which contains the two primer sequences close enough together so that overlapping could occur. When using PCR as an analytical technique, this disadvantage is addressed with several primer sets. The probability of several very different primers successfully amplifying in an unknown material is, statistically speaking, correspondingly lower.

If the signals from PCR amplification are recorded in real time cycle by cycle (RT-PCR), the measurement can be calibrated by means of calibration curves and quantitative results can thus be obtained (qPCR or qRT-PCR). This technique is now very mature and serves as a reference standard for other measuring techniques such as DNA arrays. Using quantitative RT-PCR it is also possible to construct very precise expression profiles. However, the number of analyses is limited by the costs and the throughput. The company Applied Biosystems is once again the market leader with a system that performs 384 parallel qRT-PCR reactions in 3 hours, which can be used for genotyping or for expression profiling. In terms of availability and costs, all PCR applications are limited by the oligonucleotides that are used as primers.

### 2.3 Highly-Parallel “Sequencing by Synthesis” Methods

There are various methods for the sequence analysis of short nucleic acid-segments with very high throughputs. These methods were as a rule developed as cost-favorable alternatives to Sanger sequencing, to obtain rapid access to new genome sequences. The basic principle is the production and immobilization of a very large number of primer/template complexes, which are then processed with a DNA polymerase. Immobilization can be effected on flat chips or beads. Then by stepwise insertion of dNTPs (deoxynucleoside triphosphates) and generation of an optical signal that depends on the insertion, the sequence of the individual templates in regions of a few nucleotides is elucidated. The individual sequences are then assembled by computer-assisted evaluation and in this way the elucidation of longer, continuous sequence regions is attempted. The process can be preceded by amplification of the gene segments to be investigated, for example as in the test systems developed by 454 Life Sciences or Solexa (Bennett S. T., Barnes C., Cox A., Davies L., Brown C., Pharmacogenomics 2005 June; 6(4):373-82. Warren R. L., Sutton G. G., Jones S. J., Holt R. A., Bioinformatics 2006 Dec. 8; [Epub ahead of print]. Bentley D. R. Curr Opin Genet Dev, 2006 December; 16(6):545-52. Bennett S., Pharmacogenomics 2004 June; 5(4):433-8. Margulies, M. Eghold, M. et al. Nature 2005 Sep. 15; 437(7057):326-7. Patrick Ng, Jack J. S. Tan, Hong Sain Ooi, Yen Ling Lee, Kuo Ping Chiu, Melissa J. Fullwood, Kandhadayar G. Srinivasan, Clotilde Perbost, Lei Du, Wing-Kin Sung, Chia-Lin Wei and Yijun Ruan, Nucleic Acids Research, 2006, Vol. 34, No. 12. Robert Pinard, Alex de Winter, Gary J Sarkis, Mark B Gerstein, Karrie R Tartaro, Ramona N Plant, Michael Egholm, Jonathan M Rothberg, and John H Leamon, BMC Genomics 2006, 7:216. John H. Leamon, Michael S. Braverman and Jonathan M. Rothberg, Genes Therapy and Regulation, Vol. 3, No. 1 (2007) 15-31).

Alternatively, methods were developed that aimed at analysis of an individual molecule without prior amplification (Helicos Biosciences). Generally the signal can be generated by luminescence as a function of pyrophosphate formation during insertion (454 Life Sciences), similarly to the well-known pyrosequencing technology. Alternatively fluorescence-labeled dNTPs are used, which contain a 3 ′-OH protective group, which prevents further extension after an individual insertion. After insertion of a dNTP, the fluorescence present on the primer/template complex is detected and then the 3′-OH protective group and the fluorophor are cleaved, so that a new cycle of insertion, detection and cleavage can take place. During this, all four dNTPs with different fluorophors can be offered in parallel, or a single one sequentially in each case; in this case only detection of coloration is required.

### 2.4 DNA Microarrays

The most widely used method for expression profiling employs DNA arrays. On these arrays, short DNA or RNA segments are bound positionally resolved in rows and columns or are synthesized in situ. One or more oligos are used for each gene whose expression is to be analyzed. As in PCR, several oligos increase the level of significance of the method. Other parameters for the quality of the array measurement are the oligo quality, length, and sequence selection, and the execution of the hybridization reaction. Such arrays are available for all known genes of the human genome and for some other important model organisms. There are also various theme arrays, on which there are oligos that encode genes that are ascribed to a function or a clinical picture.

As a rule the sample material that is to be investigated with an array must be amplified by PCR. For this, a generic PCR is used, in which all genes expressed in the sample are amplified starting from the universal 3′ end. This complete method makes it possible to amplify a large number of genes with only one PCR reaction and then carry out gene-specific detection with the DNA array.

When using arrays for genotyping the main problem is in sample amplification. As the positions to be investigated lie in different regions of a genome, with a PCR reaction it is only possible to amplify one or a few measuring points. As the costs for a primer set are considerable, this greatly limits the use of arrays in the area of genotyping, as the costs for the preceding PCR reactions very quickly exceed the costs for the array-based analysis. Applied Biosystems therefore also addresses these segments with a parallel PCR system. The companies Roche and Affymetrix launched a first genotyping product in the year 2004, which was approved for diagnostics. In the Amplichip, for each PCR as many SNPs as possible are measured by means of a DNA array, so that the product can still be used economically. On technical-economic grounds, however, wide application of this procedure seems rather unlikely. The availability of the individual oligos as primers is still a bottleneck.

A person skilled in the art is familiar with the production of microarrays by in-situ synthesis (matrix arrangement of the array). The system most widely used is in-situ synthesis in an array arrangement of synthetic nucleic acids or oligonucleotides. This is carried out on a substrate which is loaded by the synthesis with a large number of different polymers. The great advantage of the in-situ synthesis techniques for microarrays is the provision of a large number of molecules of different and defined sequence at addressable locations on a common support. The synthesis then has recourse to a limited set of starting materials (in DNA microarrays as a rule the 4 bases A, G, T and C) and from these it constructs any sequences of the nucleic acid polymers.

The individual molecular species can be demarcated on the one hand by separate fluidic compartments when adding the synthesis starting materials, as is the case for example in the so-called in-situ spotting method or piezoelectric techniques, which are based on inkjet printing technology (A. Blanchard, in Genetic Engineering, Principles and Methods, Vol. 20, Ed. J. Sedlow, p. 111-124, Plenum Press; A. P. Blanchard, R. J. Kaiser, L. E. Hood, High-Density Oligonucleotides Arrays, Biosens. & Bioelectronics 11, p. 687, 1996).

An alternative method is positionally resolved activation of synthesis sites, which is possible e.g. by selective illumination or selective addition of activating reagents (deprotecting agents). The number of molecules of a species synthesized in the existing known methods is relatively small, because in a microarray by definition in each case only small reaction regions are provided for one sequence in each case, so as to be able to map as many sequences as possible in the array and therefore for the functional application.

Examples of the methods already known are photolithographic light-assisted synthesis [McGall, G. et al; J. Amer. Chem. Soc. 119; 5081-5090; 1997], projector-based light-assisted synthesis [PCT/EP99/06317], fluidic synthesis by separation of the reaction spaces, indirect projector-based light-controlled synthesis by means of photo-acids and suitable reaction chambers in a microfluidic reaction support, electronically induced synthesis by positionally resolved deprotection on individual electrodes on the support and fluidic synthesis by positionally resolved deposition of the activated synthetic monomers.

### 2.5 MicroRNAs

MicroRNAs (miRNAs) are RNA molecules with a length of approx. 22 nucleotides and represent the largest group of small RNAs in plants and animals.

Approximately 250 miRNAs in the human genome are known at present, but bioinformatic methods predict a far larger number. According to various calculations miRNAs presumably regulate more than 30% of all human genes and accordingly they are involved in various ways in the most varied of processes such as the development of cancer through the control of transposon relocations, stem cell biology or muscle and brain development.

miRNAs are cut out of primary transcripts (pri-miRNAs) through two steps of endoribonuclease-III processing, first with Drosha, which produces hairpin-shaped pre-miRNA, then with Dicer, which produces siRNA-like double-stranded complexes. Mature miRNAs can then interfere in gene regulation by various mechanisms, for instance by controlling mRNA digestion or by binding to the UTR regions.

Various methods for the detection of small RNAs such as miRNAs are known by a person skilled in the art. A simple method is for example classical Northern Blot hybridization, which in addition to the sequence also provides information on the length of an RNA, but is laborious and can only be carried out at low throughput. Another gel-based method with the corresponding disadvantages is “RNase protection assay” (RPA).

A primer extension reaction can also be used for detection. In this, a labeled primer is hybridized to the miRNA, lengthened with a polymerase and the reaction mixture is separated and analyzed by gel electrophoresis.

Methods that are much quicker and more suitable for quantitative detection are based on PCR. Reverse transcription/PCR can be used, when primers with a loop are employed, which introduce a universal sequence. This universal sequence is then employed for the PCR.

Another approach for the detection and quantification of miRNAs is the use of microarrays. Particular challenges then arise from the small length of the miRNAs both for probe design and for the labeling protocols. A great variety of methods for labeling are known by a person skilled in the art, both by direct labeling with biotin or fluorophors or indirectly during cDNA synthesis or during amplification. Both chemical and enzymatic methods are known for this, e.g. based on cisplatin compounds, periodate-hydrazine labeling, T4 RNA-ligase, poly(A) polymerase or coupling to aminomodified RNAs. With respect to probe design, particular challenges arise from the small length of miRNAs mainly with respect to the signal strength owing to low duplex melting points. For this it is possible to employ modified nucleotides or use tandem probes or probes with more than 2 binding sites for miRNAs.

### 2.6 PCR on Surfaces

For various methods of nucleic acid analysis known by a person skilled in the art, for example sequencing-by-synthesis methods for whole-genome sequencing or sequencing of large sequence segments, but also for many other methods, it is necessary to generate PCR products on surfaces. In particular, bead systems (e.g. 454 Life Sciences sequencer) or array surfaces (Illumina-Solexa) are employed for this. As it is scarcely possible at present to produce a large number of special primer sequences for experimental purposes at an economic price, these methods are restricted to universal primer sequences, which have to be introduced into the nucleic acid segments that are to be investigated. As a result, the individual sequences in a sequence mixture cannot be selected specifically. A task such as the targeted sequencing of individual segments of a genome is therefore only possible if specific oligonucleotides are generated beforehand, which can be used e.g. as primers.

For the parallel amplification of a large number of different sequences it is, moreover, necessary to create special preconditions, to prevent cross-reaction during PCR. This can be effected for example by enclosing individual beads, which only bear one target sequence, in droplets of a water-in-oil emulsion, or by spatial isolation on the surfaces of chips.

At the present state of the art there is a large demand for methods for generating a large number of oligonucleotide sequences and their simultaneous use under spatial isolation of individual PCRs within a complex sample mixture of template molecules.

### 2.7 Pathogens

In recent years there have been tremendous advances in molecular diagnostics with respect to the detection, quantification and genotyping of microorganisms and viruses.

Intensive research on genomes of pathogenic organisms has driven forward the use of their DNA/RNA as analytical target molecules and has reduced the share of phenotypic assays in this field.

Various methods are currently used as genotype-based methods. Direct hybridizations with labeled oligonucleotides are used for culture analyses.

Fluorescence in situ hybridization (FISH) is an attractive method for the detection and identification of microorganisms directly from smears. However, these methods are not sensitive and are therefore restricted to very common nucleic acid molecules, e.g. ribosomal RNAs.

Array-based methods can be employed for the analysis of a large number of target molecules, but as a rule amplification and labeling of the target molecules are required.

The introduction of homogeneous methods of detection, which integrate labeling and amplification, has contributed greatly to the acceptance of molecular-diagnostic methods. In particular, quantitative real-time PCR is now a widely disseminated method. Various techniques have also found application as signal-emitting methods, e.g. TaqMan Probes, Molecular Beacons, Scorpion Primer, Sunrise Primers, DNA-Intercalators or Minor Groove Binder. These methods permit in particular the detection of less common nucleic acids in sample mixtures, for instance for the detection of low concentrations of viruses.

Sequencing-based methods are also used, but are generally restricted to common nucleic acids such as ribosomal RNA.

In addition to the detection and identification of microorganisms with respect to their genus or species, more exact genotyping is necessary for efficiently combating pathogens, monitoring populations and assessing epidemiological risks.

The most widely used methods in this direction are macro-restriction analyses of whole genome DNA or PCR-based methods for genome typing. Other known examples are pulse-field gel electrophoresis (PFGE) and ribotyping.

### 3 OBJECT OF THE INVENTION AND THE PROBLEM IT SOLVES

The invention is based on the problem of simplifying the molecular-biological processing of a mixture of biological samples, such as proteins and nucleic acids, and being able to carry out more than one molecular-biological process step, without requiring expensive purification of the samples and transfer from one reaction vessel to the next.

In particular the invention relates to improved molecular-biological processing equipment.

The object of the invention is therefore improved molecular-biological processing equipment and an improved method of processing biological samples. This invention combines the preparation of biologically functional molecules such as nucleic acids and peptides and of derivatives or analogs of these two classes of molecules in miniaturized flow cells with the sequential addition of reagents or fluids and is used for the processing of biological samples, such as proteins, nucleic acids, biogenic small molecules, for example metabolites, viruses or cells, which are fed into the miniaturized flow cells for this purpose. The material to be processed is held, through several process steps, bound in an essentially unaltered reaction space, whose prior adaptation to specific samples takes place by a positionally resolved and/or time-resolved immobilization of biologically functional molecules such as nucleic acids, peptides and derivatives or analogs thereof in the miniaturized flow cells in an arrangement as a microarray.

In a preferred embodiment the molecular-biological processing equipment according to the invention offers a method of improved analysis of sequence, chemical or biochemical modification and quantity of nucleotide sequences. This is achieved by combining selective spatially resolved binding of the analytes on an array of hybridizable probes synthesized in the miniaturized flow cells and optional sequence-nonspecific or sequence-specific amplification, in particular DNA amplification. For this the method employs one to several washing-separation steps and amplification steps. The hybridizable probes synthesized in the miniaturized flow cell can be modified chemically or biochemically for this purpose. All steps of the method can, in a preferred embodiment, optionally be monitored optically, e.g. by a flat detector which is therefore parallel or essentially parallel to the array. While the reaction cycles are proceeding or thereafter, an optically detectable result, e.g. the localization and quantity of optical markers, e.g. fluorescence markers, can be recorded.

The processing equipment according to the invention preferably has one or more heating elements, which can increase the temperature in one or more flow cells, and preferably has one or more cooling elements, which can lower the temperature in one or more flow cells.

In the equipment according to the invention the various steps of the cycle can be automated or partially automated. Therefore it offers substantial improvements over the prior art. In another preferred embodiment the molecular-biological processing equipment according to the invention permits methods for improved analysis of sequence-specific binding and/or modification events between proteins and the hybridizable probes synthesized in the miniaturized flow cell. The hybridizable probes synthesized in the miniaturized flow cell can be modified chemically or biochemically for this purpose. For this, the method uses one to several washing-separation steps. All steps of the method can, in a preferred embodiment, optionally be monitored optically, e.g. by a flat and thus essentially parallel detector. While the cycles are running or thereafter, an optically detectable result, e.g. the localization and quantity of optical markers, e.g. fluorescence markers, can be recorded.

### 4 BRIEF DESCRIPTION OF THE DRAWINGS

FIG. 1 shows an embodiment of the invention in which probe molecules 1, to which molecules to be analyzed 2b bind, were synthesized on the reaction support. Starting from the probe molecule 1, a polymerase 4b synthesizes the respective complementary strands of the molecules to be analyzed.

FIG. 2 shows an embodiment of the invention in which probe molecules 1, to which a molecule 6 was anchored, were synthesized on the reaction support. An adapter molecule 7b was anchored to molecule 6. Starting from a primer 8b that has bound to molecule 6, a polymerase 4 synthesizes, from building blocks 9a, the strand complementary to molecule 6. Building blocks 9a carry a signal-emitting group, which can be removed during insertion or thereafter, so that the strand formed contains linked building blocks 9b with a signal-emitting group or linked building blocks 9c with signal-emitting group removed.

FIG. 3 shows a table of polymerases, whose suitability for use in the processing equipment according to the invention and the methods according to the invention was investigated.

FIG. 4 shows a fluorescence image of a reaction support, on which DNA probe molecules were synthesized, which are linked to the surface via the 5′ end and have a free 3′-OH end. The image was recorded after hybridization of the reaction support with a PCR product and incubation with various polymerases, dNTPs and dNTPs with signal-emitting groups in a suitable reaction buffer. Polymerases used, from left to right: T7 DNA polymerase, Sequenase, Phi29, T4 DNA polymerase, Klenow fragment, Klenow fragment exo- and Bst DNA polymerase.

FIG. 5 shows a fluorescence image of a reaction support, on which DNA probe molecules were synthesized, which are linked to the surface via the 5′ end and have a free 3′-OH end. The image was recorded after hybridization of the reaction support with a PCR product and incubation with various polymerases, dNTPs and dNTPs with signal-emitting groups in a suitable reaction buffer. Polymerases used, from left to right: Taq DNA polymerase, 9° N, Vent DNA polymerase, Vent-DNA polymerase, Pfu DNA polymerase, Therminator, Phusion Hotstart.

FIGS. 6A to 6F show fluorescence values (arbitrary units) of a reaction support with self-complementary hairpin probes synthesized on the surface after extension by various polymerases with insertion of signal-emitting groups and subsequent fluorescence detection. For this, inverse probes (5′-3′ synthesis) with a length between 27 and 30 nucleotides were proposed, which pair with themselves via a T-tetraloop (see FIG. 6G). The DNA polymerases used are: FIG. 6A Vent; 6B Vent-; 6C, Pfu; 6D Therminator, 6E Phusion Hotstart, FIG. 6F Klenow fragment E. coli DNA polymerase I. Various sequences were tested for the pairing region (stem) and the length of the stems was varied between 4 and 7 nucleotides (4stem-7stem). Furthermore, various single-strand template sequences were tested, which were to be copied by the respective DNA polymerase. In this template sequence it was coded, by the presence of 1-3 adenosine nucleotides, for the insertion of 1-3 biotin-labeled dUTP, in order to test the dependence of the fluorescence on the number of biotins incorporated (see 6F, 1 Bio-3 Bio). 6F shows, as an example, data for reactions with the 3′-5′-exonuclease-deficient Klenow fragment of E. coli DNA polymerase I (KF-). It can be seen that there is an increase in extension efficiency with increasing stem length. The increase in fluorescence as a function of the number of encoded biotin markers is almost linear.

FIG. 7 shows a fluorescence image of a reaction support, on which DNA probe molecules were synthesized, which are linked to the surface via the 3′ end and were hybridized to the primers. The primers then bind at the end of the probe-molecule that is nearer to surface of the reaction support (i.e. proximal end). The image was recorded after incubation with various polymerases, dNTPs and dNTPs with signal-bearing groups in a suitable reaction buffer. Polymerases used, from left to right: T7 DNA polymerase, Sequenase, Phi29, T4 DNA polymerase, Klenow fragment, Klenow fragment exo- and Bst DNA polymerase. The dark-shaded arrow indicates the direction of linkage of building blocks by the polymerase.

FIG. 8 shows the reaction support from FIG. 7 after washing with water.

FIG. 9 shows the reaction support from FIG. 8 after re-hybridization of primers onto the DNA probe molecules and incubation with various polymerases, dNTPs and dNTPs with signal-bearing groups in a suitable reaction buffer. The polymerases used in this second transcription operation are, from left to right: Taq DNA polymerase, 9° N, Vent DNA polymerase, Vent-DNA polymerase, Pfu DNA polymerase, Therminator, Phusion Hotstart.

FIG. 10 shows two variants of the preferred embodiment “on-chip ligation”. In this preferred embodiment there is linkage of two probe molecules, depending on a sample molecule to be analyzed. The dark-shaded arrow indicates the site of linkage, i.e. ligation. The ligation can take place e.g. enzymatically or chemically.

FIGS. 11A and 11B show two variants of the preferred embodiment “PCR-on-chip”. Both variants have in common the use of a locus-specific (and allele-specific) probe, which was synthesized on the reaction support and was hybridized with the sequence region to be analyzed of the sample molecule. In the variant shown in FIG. 11A a so-called “Universal Tag” is added covalently onto the sample molecule to be analyzed and/or to be amplified. Furthermore, a “Universal primer” is used, which is complementary to this “Universal Tag”. Amplification takes place between the universal primer and the locus-specific (and allele-specific) probe. A “Universal Tag” is not used in variant 11B. The universal primer used here is a mixture of so-called random primers, which bind at different sites of the sample molecule to be analyzed and/or to be amplified. Amplification takes place between the respective binding site of the universal primer and the locus-specific (and allele-specific) probe.

FIG. 12 shows an embodiment in which reverse transcription and PCR are combined. Amplification then takes place finally between the poly(A) region of the cDNA formed and a locus-specific probe, which has been synthesized on the reaction support.

FIG. 13 shows the preferred embodiment “microRNA capture-signal amplification”. In this, microRNA is bound by probe molecules. MicroRNA can then, or also previously, be labeled with a universal sequence, e.g. an adenine sequence (poly(A) tail). This sequence can be used as primer, for copying a circular DNA with a sequence binding to the primer, in a reaction that is known by a person skilled in the art as “Rolling circle amplification”. The resultant DNA can be labeled differently for detection.

FIG. 14 shows data from experiments relating to an embodiment of the invention for the analysis of microRNAs (miRSNA). The diagram in FIG. 14A shows scatter-plots, which show the reproducibility of the signal intensities of the individual spots on microarrays used for two hybridizations of miRNAs from a human heart sample (top), or the differences in the signal intensities between two hybridizations, when samples from different tissues are compared (heart and brain, bottom diagram). FIG. 14B shows two bar charts, which show the signal intensities of various microarray probes after hybridization with an miRNA sample from the human brain, which were designed for the detection of a particular miRNA. The probes are either completely complementary to the miRNA (PM) or carry one, two or three mismatches (MM, single, double, triple). In addition, intensities of analogous probes are shown, which have two successive complementary sequences of miRNA, which either follow one another directly or are separated by a spacer: tandem or tandem with spacer). The bar chart bottom left shows analogous data for another miRNA. This is expressed differently in brain and heart tissue; the signal intensities for the hybridizations of the samples from the respective tissues are compared.

FIG. 15 shows a fluorescence image of a microarray of hybridizations of miRNAs from various tissues (heart and brain) that were hybridized under various conditions, as shown in the table at the bottom.

FIG. 16 shows a fluorescence image of a microarray after hybridization with miRNAs and labeling with biotin/streptavidin-phycoerythrin (before signal amplification; recording time: 2780 ms).

FIG. 17 shows a fluorescence image of a microarray after hybridization with miRNAs and labeling with biotin/streptavidin-phycoerythrin (SAPE) and subsequent signal amplification by means of an antibody, which in its turn is biotin-labeled and re-labeling with streptavidin-phycoerythrin (recording time: 1500 ms)

FIG. 18 shows data on the dependence of the signal intensity of the fluorescence signals of array images after hybridization with RNA samples from human brain, which was performed either without prior purification or after selective enrichment of the miRNAs. (Starting material: 5 μg brain-RNA; labeled with mirVANA labeling kit (Ambion); the data were corrected for the background signals and are without “spiked” controls but with original and tandem probe sequences; normalization was not carried out.)

FIG. 19 shows data and a schematic for the theme complex “Analysis of single-nucleotide substitutions for SNP genotyping, resequencing or methylation analysis”. A diagram explaining the assay principle is shown top right. Depending on the nucleotide of the sample molecule at a particular position, a more or less efficient primer extension by a DNA polymerase takes place on different primer molecules located on the surface. A fluorescence image of a microarray after primer extension as described is shown on the left. During extension, biotin was incorporated, and was then labeled with streptavidin-phycoerythrin. The signal differences for different nucleotide pairs in the primer are clearly discernible in the enlargement. A bar chart showing quantitatively the fluorescence signals of a corresponding array is shown bottom right. (PM=perfect match of the nucleotide pair in the primer, MM=mismatch).

FIG. 20 shows data for the theme complex “PCR-on-chip”. PCR reactions were carried out in the reaction support corresponding to the two schemes with a PCR product of the GFP gene as template, with in each case one of the primers immobilized on the surface. During the reaction biotin was incorporated and labeled using SAPE. Fluorescence images of the arrays are in each case shown to the right of the respective schemes. The data points designated with PCR are from a PCR reaction, and the images designated PEX underwent the same incubations at the same temperatures, but not cyclically.

FIG. 21 shows data for the theme complex PCR-on-chip. PCR reactions were carried out in the reaction support corresponding to the scheme on the left with a PCR product of the GFP gene as template, with both primers (GFPforw01 and GFPrev01) immobilized separately on the surface within one reaction support. Various primer lengths between 10 and 30 nucleotides were used. During the reaction, biotin was incorporated and was labeled using SAPE. Fluorescence images of the arrays are in each case shown to the right of the schemes. Identical PCR reactions were used in two identical reaction supports, with exclusively GFPforw01 in one, and exclusively GFPrev01 in the other, added as soluble primer. Efficient signal generation by the amplification is only observed in the positions where a PCR-capable, oppositely directed primer pair is achieved.

FIG. 22 shows data for the theme complex “PCR-on-chip”. PCR reactions were carried out in the reaction support with a PCR product of the GFP gene as template, where there was a great variety of primers immobilized on the surface within one reaction support and in each case a primer (GFPforw01 and GFPrev01) was added. As shown in the diagram at the bottom, in each case 30 different immobilized primers were used in the sense and antisense direction. As a result, 30 different PCR products of varying length are formed in each array. Depending on which primer is added in soluble form, product formation is only observed for the sense or antisense primers.

FIG. 23 shows data relating to “on-chip primer extension” for the copying of oligonucleotides synthesized in the reaction support and immobilized. In accordance with the scheme at the bottom of the diagram, primers are hybridized to the oligonucleotides and extended by a polymerase. The resultant, noncovalently bound single strands can then be removed from the reaction support by washing, and can be used as a template in a PCR, which can be used for their amplification.

FIG. 24 shows a scheme, which shows a so-called “Strand Displacement Amplification” in the reaction support. A hairpin probe with a free 3′-nucleotide is synthesized in the reaction support, and contains a recognition sequence for a more remotely cutting Nicking Endonuclease in the double-stranded region. After primer extension by a polymerase, the newly formed strand is cut by the nicking endonuclease and is now available for a repeat primer extension. Both enzymes, polymerase and nuclease, can be present in the solution simultaneously, and can bring about isothermal, linear amplification.

FIG. 25 shows a scheme, which shows a so-called “Strand Displacement Amplification” in the reaction support. A probe synthesized in the reaction support is hybridized with a primer, so that a recognition sequence for a more remotely cutting Nicking Endonuclease is formed in the double-stranded region. The primers can optionally be linked chemically to the probe synthesized in the reaction support. After primer extension by a polymerase, the newly formed strand is cut by the nicking endonuclease and is now available for a repeat primer extension. Both enzymes, polymerase and nuclease, can be present in the solution simultaneously and can bring about isothermal, linear amplification.

FIG. 26 shows a scheme in which amplification takes place on the surface of the reaction support. Two adjacent probe molecules with different sequence (primer A and primer B) cannot be extended template-dependently by a polymerase, as they are too far apart to bind to one another (no formation of primer homo- or hetero-dimers known by a person skilled in the art). If soluble molecules that are not attached to the surface of the reaction support are added, the primer can bind selectively to desired molecules from a complex mixture of molecules (e.g. DNA fragments from genomic DNA) and are extended by the polymerase. They then reach a length that permits binding of an adjacent primer, so that the latter can also be extended by the polymerase. After an initial extension step the reaction support is washed under stringent conditions, so that all molecules and ions not bound covalently to the surface of the reaction support are removed from the reaction support. After again adding reagents that are necessary for a PCR reaction known by a person skilled in the art, the reaction support is submitted to a temperature-time profile that makes a PCR reaction possible.

FIG. 27 shows a scheme in which amplification takes place on the surface of the reaction support. Two adjacent probe molecules with different sequence (primer A and primer B) cannot be extended template-dependently by a polymerase, as they are too far apart to bind to one another (no formation of primer homo- or hetero-dimers known by a person skilled in the art). If soluble molecules that are not linked to the surface of the reaction support are added, the primers can bind. A hybridization and a washing profile are now carried out, permitting the selective binding of desired molecules from complex sample mixtures (e.g. fragments from genomic DNA). Unwanted molecules are removed from the reaction support by washing. The primers that have bound molecules are extended by the polymerase, after adding the reagents necessary for PCR. They then reach a length that permits binding of an adjacent primer, so that the latter can also be extended by the polymerase. After an initial extension step the reaction support is optionally washed under stringent conditions, so that all molecules and ions not bound covalently to the surface of the reaction support are removed from the reaction support. After again adding reagents that are necessary for a PCR reaction known by a person skilled in the art, the reaction support is submitted to a temperature-time profile that makes a PCR reaction possible.

FIG. 28 shows a principle for the detection of microRNAs. These bind selectively to probe molecules synthesized in the reaction support and can, by hybridization and washing steps known by a person skilled in the art, be retained selectively in the reaction support out of a complex sample mixture. Unwanted molecules are removed by washing. Optionally, a single-strand-specific (ssDNA) nuclease is now added, which processes all unbound, single-stranded probe molecules. The probe molecules bound to microRNAs are not processed. The bound microRNAs now function as primers and are extended by a polymerase, incorporating building blocks with signal-emitting groups or haptenes. After washing, these can be detected directly or after binding a haptene-specific ligand, which in its turn contains one or more signal-emitting groups.

FIG. 29 shows a principle for the detection of microRNAs. These bind selectively to probe molecules synthesized in the reaction support and can, by hybridization and washing steps known by a person skilled in the art, be retained selectively in the reaction support out of a complex sample mixture. Unwanted molecules are removed by washing. The microRNAs were labeled beforehand with one or more signal-emitting groups or haptenes. After washing, these can be detected directly or after binding a haptene-specific ligand, which in its turn contains one or more signal-emitting groups.

FIG. 30 shows a principle for the detection of microRNAs. These bind selectively to probe molecules synthesized in the reaction support and can, by hybridization and washing steps known by a person skilled in the art, be retained selectively in the reaction support out of a complex sample mixture. Unwanted molecules are removed by washing. The microRNAs were labeled beforehand with one or more signal-emitting groups or haptenes. After washing, these can be detected directly or after binding a haptene-specific ligand, which in its turn contains one or more signal-emitting groups.

FIG. 31 shows a principle for the detection of microRNAs. These bind selectively to probe molecules synthesized in the reaction support and can, by hybridization and washing steps known by a person skilled in the art, be retained selectively in the reaction support out of a complex sample mixture. In this case the probe molecules contain several sites for the binding of a microRNA, preferably two, three, four or five. Unwanted molecules are removed by washing. The microRNAs were labeled beforehand with one or more signal-emitting groups or haptenes. After washing, these can be detected directly or after binding a haptene-specific ligand, which in its turn contains one or more signal-emitting groups.

FIG. 32 shows a principle for the detection of microRNAs. These bind selectively to probe molecules synthesized in the reaction support and can, by hybridization and washing steps known by a person skilled in the art, be retained selectively in the reaction support out of a complex sample mixture. In this case the probe molecules contain several sites for the binding of a microRNA, preferably two, three, four or five. Unwanted molecules are removed by washing. The microRNAs were labeled beforehand with one or more signal-emitting groups or haptenes. After washing, these can be detected directly or after binding a haptene-specific ligand, which in its turn contains one or more signal-emitting groups.

FIG. 33 shows a principle for the detection of microRNAs. These bind selectively to probe molecules synthesized in the reaction support and can, by hybridization and washing steps known by a person skilled in the art, be retained selectively in the reaction support out of a complex sample mixture. Unwanted molecules are removed by washing. The microRNAs are then labeled by one or more enzymes, preferably polymerases and/or ligases, with one or more signal-emitting groups or haptenes. After washing, these can be detected directly or after binding a haptene-specific ligand, which in its turn contains one or more signal-emitting groups.

FIG. 34 shows a principle for the detection of microRNAs. These bind selectively to probe molecules synthesized in the reaction support and can, by hybridization and washing steps known by a person skilled in the art, be retained selectively in the reaction support out of a complex sample mixture. Unwanted molecules are removed by washing. The microRNAs are then labeled by one or more enzymes, preferably polymerases and/or ligases, with one or more signal-emitting groups or haptenes. After washing, these can be detected directly or after binding a haptene-specific ligand, which in its turn contains one or more signal-emitting groups.

FIG. 35 shows a principle for the detection of microRNAs. These bind selectively to probe molecules synthesized in the reaction support and can, by hybridization and washing steps known by a person skilled in the art, be retained selectively in the reaction support out of a complex sample mixture. In this case the probe molecules contain several sites for the binding of a microRNA, preferably two, three, four or five. Unwanted molecules are removed by washing. The microRNAs are then labeled by one or more enzymes, preferably polymerases and/or ligases, with one or more signal-emitting groups or haptenes. After washing, these can be detected directly or after binding a haptene-specific ligand, which in its turn contains one or more signal-emitting groups.

FIG. 36 shows a principle for the detection of microRNAs. These bind selectively to probe molecules synthesized in the reaction support and can, by hybridization and washing steps known by a person skilled in the art, be retained selectively in the reaction support out of a complex sample mixture. In this case the probe molecules contain several sites for the binding of a microRNA, preferably two, three, four or five. Unwanted molecules are removed by washing. The microRNAs are then labeled by one or more enzymes, preferably polymerases and/or ligases, with one or more signal-emitting groups or haptenes. After washing, these can be detected directly or after binding a haptene-specific ligand, which in its turn contains one or more signal-emitting groups.

FIG. 37 shows a flowchart for the detection and typing of viruses and other pathogens. After quantitative real-time PCR in the case of a positive test the resultant PCR product is used directly, without repeat PCR for hybridization in the reaction support. This serves for the typing of the detected virus or for the discovery of new mutants, strains or types of a virus.

FIG. 38 shows an embodiment in which a probe molecule, synthesized in the reaction support of the processing equipment according to the invention, forms a hairpin structure. There are preferably two possible recognition sequences in the stem of the hairpin structure: one near the surface (i.e. proximal) and one remote from the surface (i.e. distal).

FIG. 39 shows an embodiment in which a probe molecule, which forms a hairpin structure and has two sequences A and A*, which are connected by a linker, is used for binding a sequence A (figure A) or A* (figure B). This causes a change in the secondary structure of the probe molecule, which can be detected.

FIG. 40 shows an embodiment as in FIG. 39, except that in each case a sequence X and Z is added on to sequence A (figure A) and A* (figure B), moreover these do not pair with one another and have special properties, explained in more detail in the following.

FIG. 41 shows an embodiment as in FIG. 38, except that in each case a fluorophor (figure A) or a quencher molecule (figure B) is added on to sequence A and A*, which simplify detection of the change of the secondary structure in the probe molecule.

FIG. 42 shows an embodiment as in FIG. 39, which is used for binding the two strands of a double-stranded sample molecule (target).

FIG. 43 shows an embodiment in which a probe molecule, synthesized in the reaction support of the processing equipment according to the invention, forms a hairpin structure. Two possible recognition sequences are present, and the probe molecule is not linked terminally, but internally to the surface of the reaction support. In type A the recognition sequence or sequences is/are in the loop and in type B the recognition sequence or sequences is/are in the stem. The recognition sequence is in each case shown with dark shading.

FIG. 44 shows a so-called RAKE assay for the detection of miRNA (miRNA-RAKE assay). In RAKE assay (“RNA primed, array-based Klenow enzyme assay”) an array-based extension reaction is carried out with the aid of the Klenow fragment of DNA polymerase I starting from an RNA primer. In the embodiment shown in FIG. 44, the miRNA to be detected binds to a DNA probe immobilized on the surface of the array or microarray. Starting from the DNA-RNA heteroduplex, the miRNA is extended by means of Klenow enzyme and nucleotides (NTP), i.e. the miRNA functions as a primer. A proportion of the nucleotides can be replaced with labeled nucleotides, e.g. with biotin (bio) labeled nucleotides (bio-NTP), so that the miRNA can be detected. In the embodiment shown in FIG. 44, the DNA probe is immobilized with its 5′ end on the support surface and the 3′ end of the DNA probe is free. After binding of the miRNA the extension reaction therefore takes place in the direction towards the support surface. Advantages of the method shown are that the information contained in the analyte molecule (miRNA) is not copied onto the chip, so that the chip is reusable. Furthermore, the stability of the duplex of probe and miRNA is increased by the elongation.

FIG. 45 shows further details of the embodiment of the miRNA-RAKE assay shown in FIG. 44. The DNA probe consists of two regions: the first region comprises a hybridization sequence (light gray), which is reverse-complementary to the miRNA sequence to be detected; the second region comprises a labeling sequence (medium-gray). The hybridization sequence is located at the 3′ end of the DNA probe. The labeling sequence is located at the 5′ end of the DNA probe. This labeling sequence determines the insertion of the labeled nucleotides, e.g. the insertion of biotin-labeled uridine. In preferred embodiments the various DNA probes immobilized on the support surface have identical labeling sequences, but different hybridization sequences.

FIG. 46 shows a so-called “inverse” RAKE assay for the detection of miRNA. In contrast to the assay shown in FIG. 44, the DNA probe is immobilized with its 3′ end on the support surface and the 5′ end of the DNA probe is free. The hybridization sequence is located at the 3′ end of the DNA probe. The labeling sequence is located at the 5′ end of the DNA probe. After binding of the miRNA, the extension reaction therefore takes place in the direction away from the support surface. As in the method shown in FIGS. 44 and 45, this also has the advantages that the information contained in the analyte molecule (miRNA) is not copied onto the chip, so that the chip is reusable. Furthermore, the stability of the duplex of probe and miRNA is increased by the elongation.

FIG. 47 shows an inverse tandem-miRNA-RAKE assay. In this embodiment the DNA probe comprises at least three regions: two hybridization regions, which are localized immediately one after another, i.e. in tandem arrangement, at the 3′ end of the DNA probe, and a third region, which includes a labeling sequence, localized at the 5′ end of the DNA probe. This DNA probe is immobilized with its 3′ end on the support surface, whereas the 5′ end is free. In preferred embodiments the two hybridization regions have identical hybridization sequences in each case. The hybridization sequences are reverse-complementary to the miRNA to be detected. During execution of the assay, two miRNA molecules bind directly adjacent, i.e. in tandem, on the DNA probe. Owing to the binding of two miRNA molecules, the DNA-RNA heteroduplex that forms has increased stability compared with embodiments in which only one miRNA molecule can bind to the DNA probe. As in the embodiments shown in FIGS. 44 to 46, an extension reaction by Klenow enzyme and nucleotides is carried out starting from the 3′ end of an miRNA molecule hybridized to the DNA probe. A proportion of the nucleotides can be replaced with labeled nucleotides, e.g. with biotin-labeled nucleotides, so that the miRNA can be detected. As in the method shown in FIGS. 44 to 46, the information contained in the analyte molecule (miRNA) is not copied onto the chip, so that the chip is reusable. Furthermore, the stability of the duplex of probe and miRNA molecules is additionally increased by the elongation.

FIG. 48 shows a variant of the RAKE assay, in which a ligation reaction is used. This assay is designated in the present application as RALE assay (“RNA-primed, array-based ligase enzyme assay”). In the RALE assay, a DNA probe which comprises two hybridization regions is immobilized on the support surface: the first hybridization region at the 3′ end of the DNA probe comprises a hybridization sequence that is reverse-complementary to the miRNA to be detected; the second hybridization region at the 5′ end of the DNA probe comprises a hybridization sequence that is reverse-complementary to a ligation probe. During execution of the RALE assay, the miRNA to be detected and the ligation probe bind to the DNA probe. The ligation probe has a free 5′-phosphate group at its 5′ end. In addition it has a marker, e.g. a fluorescence marker, which is preferably located at the 3′ end of the ligation probe. After hybridization of miRNA and ligation probe to the immobilized DNA probe, by means of an added ligase the 3′ end of the miRNA is bound covalently to the 5′ end of the ligation probe. The miRNA is detected from the marker present in the ligation probe. In a second embodiment (not shown) the two hybridization regions on the DNA probe are exchanged, i.e. the first hybridization region is localized at the 5′ end of the DNA probe and the second hybridization region is localized at the 3′ end of the DNA probe. In this second embodiment, after hybridization of the miRNA and the ligation probe to the DNA probe, the 5′ end of the miRNA is bound covalently to the 3′ end of the ligation probe. In the second embodiment the marker is preferably located at the 5′ end of the ligation probe.

FIG. 49 shows a variant of the inverse tandem-miRNA-RAKE assay shown in FIG. 47. In this case, between the step of hybridization of the two miRNA molecules to the DNA probe and the elongation step, another process step takes place, in which the two miRNA molecules are bound to one another covalently by a ligase. The additional ligation step leads to further stabilization of the heteroduplex of DNA probe and miRNA molecules. As in the method shown in FIG. 47, the information contained in the analyte molecule (miRNA) is not copied onto the chip, so that the chip is reusable.

FIG. 50 shows an enzyme-free method of detection for miRNA molecules. In this assay a DNA probe which comprises two hybridization regions is immobilized on the support surface: the first hybridization region of the DNA probe comprises a hybridization sequence that is reverse-complementary to the miRNA to be detected; the second hybridization region of the DNA probe comprises a hybridization sequence, which is reverse-complementary to a so-called helper-oligo. This helper-oligo is a short RNA oligonucleotide with a length of 10 to 25 nucleotides, which has a marker, e.g. biotin. In a first process step, miRNA and helper-oligo hybridize to the immobilized DNA probe. In a second process step an activated nucleotide is added, which links the miRNA and helper-oligo covalently to one another in a chemical reaction. This chemical reaction is known as chemical ligation and was described for example in international patent application WO 2006/063717 (the contents of this application, with respect to chemical ligation, are fully incorporated by reference in the present application). Finally, excess nonligated helper-oligo is removed in a stringent washing step. The miRNA is detected from the marker in the helper-oligo. FIGS. 50A and 50B show two different embodiments, which differ in the orientation of the DNA probe: in the embodiment in FIG. 50A, the DNA probe is immobilized via its 3′ end on the support surface and the 5′ end is free; in the embodiment in FIG. 50B the DNA probe is immobilized via its 5′ end on the support surface and the 3′ end is free.

FIG. 51 shows a variant of the method of detection for miRNA molecules shown in FIG. 50. In this assay, a DNA probe that comprises three hybridization regions in the following order is immobilized on the support surface: the first hybridization region of the DNA probe comprises a hybridization sequence that is reverse-complementary to a first helper-oligo; the second hybridization region of the DNA probe comprises a hybridization sequence that is reverse-complementary to the miRNA to be detected; the third hybridization region of the DNA probe comprises a hybridization sequence that is reverse-complementary to a third helper-oligo. In a first process step, the first helper-oligo, the second helper-oligo and the miRNA hybridize to the DNA probe. The first and the second helper-oligos are RNA oligonucleotides with length of 10 to 25 bp. In a second process step, activated nucleotides are added, which link the miRNA covalently at one end with the first helper-oligo and at the other end with the second helper-oligo in a chemical reaction. This chemical reaction is known as chemical ligation and was described for example in international patent application WO 2006/063717. In a subsequent denaturation step, the miRNA extended by the two helper-oligos is separated from the DNA probe and is amplified in an amplification reaction (e.g. PCR or “Whole Genome Amplification” (WGA)). For this it is possible to use a primer pair in which the first primer has the same sequence as the first helper-oligo and the second primer has the sequence that is reverse-complementary to the second helper-oligo. In an alternative embodiment the first primer has the same sequence as the second helper-oligo and the second primer has the sequence reverse-complementary to the second helper-oligo. In the amplification reaction, markers can be incorporated in the amplification products, e.g. using labeled nucleotides such as bio-NTP. In a subsequent step the amplification products are hybridized back to the microarray. The miRNAs can then be detected from the marker introduced in the amplification reaction.

FIG. 52 shows a variant of the RAKE assay for the detection of miRNA. In this case two different DNA probes are immobilized on the support surface of a microarray. The first of the two DNA probes contains, at its 5′ end, the recognition sequence for an RNA polymerase, for example the T7 promoter sequence, when T7-RNA polymerase is used. At its 3′ end, this first DNA probe is reverse-complementary to an miRNA to be detected. The second DNA probe on the support surface is reverse-complementary to the first DNA probe. After hybridization of the miRNA on the first DNA probe, the miRNA is extended by Klenow enzyme and nucleotides (NTP) in an extension reaction. A proportion of the nucleotides used can carry a marker such as biotin. For example, biotin-labeled uridine-nucleotides (bio-UTP) can be used. The miRNA is detected from the inserted labeled nucleotides. After detection, selected, extended miRNA molecules can be removed from the support surface by denaturation. Reverse transcriptase (=RT-polymerase) and RNA polymerase (e.g. T7-polymerase) are added to these single-stranded DNA-RNA chimeras, which consist of the original miRNA and the DNA strand added on in the extension reaction. In the amplification reaction brought about by this enzyme, the strand reverse-complementary to the DNA-RNA chimeras is amplified linearly at about 1000-times amplification. The amplification products are hybridized back to the support surface of the microarray and in so doing they hybridize to the aforementioned second DNA probe.

FIG. 53 shows probes with a Cap group for use in the microarray systems of the invention. The probe molecules immobilized on the support surface have a Cap group at their 5′ end. During hybridization of a target molecule, e.g. an miRNA, the Cap group interacts with the duplex and increases its thermal stability. The chemical structure of an example of a Cap group is shown to the right of the diagram. Interaction of the Cap group with the duplex intensifies differences in melting point between completely Watson-Crick-paired duplexes and duplexes that contain base mismatches. This is useful in particular for differentiating miRNAs that only differ in one or a few nucleotides near the 3′ end or at the 3′ end, e.g. members of the let-7 family.

### 5 ESSENTIAL FEATURES OF THE PROPOSED SOLUTION

### 5.1 Definitions

Before describing the present invention in the detail, it is to be noted that the invention is not limited to the special, preferred methods, experimental instructions and reagents described herein, as these can vary. It is also evident that the terminology used herein only serves the purpose of describing special embodiments and is not intended to limit the scope of the present invention, which is only limited by the appended patent claims. Unless stated otherwise, all technical and scientific terms have the same meanings as they are usually understood by a person skilled in the art.

Preferably the terms used herein have the meanings assigned to them in “A multilingual glossary of biotechnological terms: (IUPAC Recommendation)”, Leuenberger, H. G. W, Nagel, B. and Kölbl, H. Eds. (1995), Helvetica Chimica Acta, CH-4010 Basel, Switzerland).

In the complete description and the following patent claims, the word “comprise” and variations such as “comprises” and “comprising”, unless the context requires otherwise, signify the inclusion of a given integer or a step or a group of integers or steps, but not the exclusion of any other integer or a step or of a group of integers or steps.

Numerous documents are cited in the complete text of this description. Each of the documents cited herein (including all patent specifications, patent applications, scientific publications, manufacturer information, instructions, deposits of sequences in GenBank under an accession number etc.), regardless of whether cited hereinbefore or hereinafter, is hereby included in its entirety by reference. Nothing in this description is to be interpreted as an admission that the present invention is not entitled to precede such a disclosure by virtue of prior invention.

A “receptor” in the sense of the present invention is any molecule that is able to bind an analyte. Preferably the binding to the analyte is specific and selective. In preferred embodiments of the invention the “receptor” is immobilized, preferably on a supporting material or simply “support”. Preferred receptors of the invention comprise oligopeptides or polypeptides, also summarized briefly with the term “peptide” in the following. These oligopeptides or polypeptides can be composed of the known, naturally occurring 20 amino acids, but they can also contain naturally occurring or synthetic amino acid analogs and/or derivatives. These amino acids, amino acid analogs and/or derivatives can optionally carry markers, for example dyes. Oligopeptides typically consist of up to 30 amino acids, amino acid analogs and/or derivatives, whereas polypeptides consist of more than 30 amino acids, amino acid analogs and/or derivatives, and there is no sharp demarcation between oligopeptides and polypeptides. Oligopeptides or polypeptides that are used as receptors in the sense of the invention preferably comprise at least 8, 9, 10, 11, 12, 13, 14, 15, 16, 17, 18, 19, 20, 25, 30, 35, 40, 45, 50, 55 or 60 amino acids, amino acid analogs and/or derivatives.

Especially preferred “receptors” of the invention comprise oligonucleotides or polynucleotides, in the following also designated together as nucleic acids. These oligonucleotides or polynucleotides preferably consist of deoxyribonucleotides or ribonucleotides or mixtures thereof and can be single-stranded or double-stranded. These oligonucleotides can furthermore contain additionally or exclusively nucleic acid analogs and/or derivatives, for example peptide nucleic acids (PNA), locked nucleic acids (LNA), etc. In preferred embodiments the nucleobases of these deoxyribonucleotides, ribonucleotides, nucleotide analogs and nucleotide derivatives are selected from adenine (A), cytosine (C), guanine (G), thymine (T) and uracil (U), where deoxyribonucleotides typically contain the nucleobases A, C, G or T and ribonucleotides typically contain the nucleobases A, C, G or U. Apart from the aforesaid nucleobases, the receptors of the invention can also contain variants and derivatives of these nucleobases, for example methylated nucleobases or those bearing covalently bound markers, for example dyes or haptenes. Oligonucleotides typically consist of up to 30 nucleotides, nucleotide analogs or derivatives, whereas polynucleotides consist of more than 30 nucleotides, nucleotide analogs or derivatives, and there is no sharp demarcation between oligonucleotides and polynucleotides. Oligonucleotides or polynucleotides used as receptors in the sense of the invention preferably comprise at least 8, 9, 10, 11, 12, 13, 14, 15, 16, 17, 18, 19, 20, 25, 30, 35, 40, 45, 50, 55 or 60 nucleotides, nucleotide analogs or derivatives.

In the following, the units linked to receptors in each case are termed “building blocks”. As a rule these are individual amino acids or individual nucleotides or nucleotide analogs. In certain embodiments a building block can, however, also consist of 2, 3, 4, 5, 6, 7, 8, 9, 10 or more amino acids, nucleotides or nucleotide analogs. In this case the synthesis time of the receptor, for example when using building blocks consisting of two nucleotides, can be halved. The free building blocks preferably have an activated or linkable group, via which the building block can be linked to the support or to a building block already previously attached to the support, and at least one activatable or deprotectable group. The term “activation” is used here in the usual sense as a modification of a chemical group, which enables this group under suitable conditions—i.e. the conditions attainable in microfluidic molecular-biological processing equipment—to form a covalent bond to another group. A great many suitable methods of activation are known in the prior art, which make it possible for example to attach nucleotides to a free OH group or amino acids to a free amino or carboxyl group.

The term “asymmetric receptors”, as used herein, designates receptors that consist of at least 2 different types of receptor building blocks, i.e. contain more than 1, 2, 3, 4, 5, 6, 7, or 8 different types of receptor building blocks. A “type of receptor building blocks” or also a “set of receptor building blocks” comprises in each case a group of receptor building blocks that have a structural feature in common, but differ in another structural feature. If the receptor consists of nucleic acids, nucleic acid analogs and/or nucleic acid derivatives, then for example a “set of receptor building blocks” comprises all deoxyribonucleotides regardless of which nucleobase is borne by the deoxyribonucleotide. A second “set of receptor building blocks” comprises for example all locked nucleic acids, i.e. all LNA-nucleotides, regardless of which nucleobase is borne by the respective LNA-nucleotide. Accordingly, this means that an “asymmetric receptor”, which consists of nucleic acids, comprises at least two different nucleotide types, for example DNA+LNA or DNA+PNA or DNA+RNA.

The receptors of the invention can form one or more “secondary structures”. A receptor of the invention can have one or more secondary structures in its entirety or also only in partial regions. For the case when the receptors of the invention are oligopeptides or polypeptides, these can form, among others, the secondary structures α-helix, β-sheet and β-turn, which are known by a person skilled in the art. These secondary structures require a minimum length of the relevant oligo- or polypeptide, for example in the case of α-helices at least 4 amino acids, in the case of β-sheets at least 4 amino acids. These secondary structures preferably comprise at least 5, 6, 7, 8, 9, 10, 11, 12, 13, 14, 15, 20, 25 or 30 amino acids. For the case when the receptors of the invention are oligo- or polynucleotides, they can have secondary structures such as hairpin structures, internal loops, so-called “bulges” and/or so-called pseudonodes. It is especially preferable for the receptor to be a single-stranded oligo- or polynucleotide and the secondary structure to be a hairpin structure. The hairpin structure is characterized by a stem, which consists of a self-complementary helix, and a loop, which consists of a single-stranded, unpaired region. Preferably the loop has a length of at least 3, 4, 5, 6, 7, 8, 9, 10, 11, 12 or more nucleotides. Furthermore it is preferable for the loop to have a length of at most 100, 90, 80, 70, 60, 50, nucleotides. The stem preferably has a length of 3, 4, 5, 6, 7, 8, 9, 10, 11, 12, 13, 14, 15 or more base pairs. Furthermore, it is preferable for the stem to have a length of at most 40, 35, 30, or 25 base pairs. The total length of the receptor, which forms a hairpin structure, is preferably at least 9, 10, 11, 12, 13, 14, 15, 16, 17, 18, 19, 20, 21, 22, 23, 24, 25, 26, 27, 28, 29, 30, 32, 35, 40, 45, 50, 60, 80, 100 or more nucleotides. It is obvious that an oligo- or polynucleotide can only form a hairpin structure when it has self-complementary regions. Methods, algorithms and computer programs for the determination of these self-complementary regions and for the construction of oligo- or polynucleotides that have hairpin structures or other secondary structures, are known by a person skilled in the art. Furthermore, experimental or mathematical methods, algorithms or computer programs for determining physical properties of said hairpin structures are known by a person skilled in the art; in particular, a person skilled in the art knows experimental or mathematical methods for determining the melting point of said hairpin structures, or more precisely the stem of the hairpin. In preferred embodiments of the invention, the melting point of the hairpin structure is lower than the melting point of the hybridization product from receptor and specifically bindable analyte. In other words: in the presence of a specifically bindable analyte the hairpin structure opens, and the receptor and the specifically bindable analyte hybridize to one another.

A “light source matrix” in the sense of this invention is preferably a programmable light source matrix, e.g. selected from a light valve matrix, a mirror array, a UV-laser array and a UV-LED-(diode)-array. The programmable light source matrix or illumination matrix can be a reflection matrix, a light valve matrix, e.g. an LCD-matrix, or a self-emitting illumination matrix. In preferred embodiments the light valve matrix can control a source of radiation, which preferably can select predetermined positions. Such light matrixes are for example disclosed in WO 00/13018. Preferably the light valve matrix is selected from the group comprising DLP, LCoS panels, and LCD panels and the source of radiation, which can select predetermined positions, is selected from an LED-array and an OLED-array.

A “miniaturized flow cell” in the sense of this invention is a three-dimensional microcavity, in each case having at least one inlet and one outlet. Preferably the interior is designed so that, like a single long channel, it leads from one or more inlets to one or more outlets and therefore permits rapid pressure-operated filling (overpressure and/or underpressure) with reagents and other media. This channel preferably has a diameter in the range from 10 to 10 000 μm, especially preferably from 50 to 250 μm and can basically be constructed in any shape, e.g. with circular, oval, square or rectangular cross-section. The length of a flow cell can vary between 10 μm and 10 cm. In the case of flow cell lengths that exceed the width or length of the support, it can also be meander-shaped.

A “primer extension reaction” in the sense of the invention denotes any reaction in which a primer molecule, which is hybridized to a template, is extended according to the sequence of the template. The template can be a nucleic acid, i.e. DNA or RNA, or a nucleic acid analog. If the template is DNA, the primer extension reaction can be accomplished with any suitable DNA-dependent polymerase known by a person skilled in the art. Preferably the DNA-dependent polymerase is a DNA polymerase, but suitable DNA-dependent RNA polymerases can also find application in the “primer extension reactions” of the invention. If the template is RNA, the primer extension reaction can be accomplished with any suitable RNA-dependent polymerase known by a person skilled in the art. Preferably the RNA-dependent polymerase is an RNA-dependent DNA polymerase. These RNA-dependent DNA polymerases are also known as “reverse transcriptases”.

“Amplification” of a nucleic acid in the sense of this invention denotes any production of a new nucleic acid strand starting from an existing nucleic acid strand. The term “amplification” therefore also includes the synthesis of a single complementary strand in a primer extension reaction. The term “amplification” preferably also comprises the doubling or further multiplication of nucleic acid strands in methods such as polymerase chain reaction or Multiple Displacement Amplification.

### 5.2 Summary of the Invention

In a first aspect the invention relates to molecular-biological processing equipment comprising (a) an apparatus for the in-situ synthesis of arrays of receptors, (b) one or more elements for the execution of fluidic steps, such as sample addition, addition of reagents, washing steps and/or sample withdrawal, (c) a detection unit for detecting an optical or electrical signal, (d) a programmable unit for controlling the synthesis, and (e) a programmable unit for controlling the fluidics, detection and the storage and management of the measurement data.

In preferred embodiments of this first aspect, the molecular-biological processing equipment is characterized in that it has one or more miniaturized flow cells and in that a fluidic step in this one or these several miniaturized flow cells takes 1 min or less, more preferably 30 s or less, still more preferably 10 s or less, still more preferably 1 s or less, still more preferably 0.1 s or less, still more preferably 0.01 s or less, still more preferably 0.001 s or less and most preferably 0.0001 s or less. In preferred embodiments of this first aspect the molecular-biological processing equipment is characterized in that it has one or more miniaturized flow cells and in that the fluid volume in this one or these several miniaturized flow cells is 40% or less, more preferably 25% or less, still more preferably 10% or less, still more preferably 5% or less, still more preferably 1% or less, still more preferably 0.5% or less, still more preferably 0.1% or less, still more preferably 0.01% or less, still more preferably 0.001% or less and most preferably 0.0001% or less of the volume of the feed line to the fluid reservoir. In preferred embodiments of this first aspect the molecular-biological processing equipment is characterized in that it has one or more miniaturized flow cells and in that in the execution of the fluidic steps at least 2, more preferably at least 5, still more preferably at least 10, still more preferably at least 100, still more preferably at least 500 and most preferably at least 1000 different reagents are brought into this one or these several miniaturized flow cells. In especially preferred embodiments, in the execution of the fluidic steps these various reagents are brought into one or more miniaturized flow cells in 10 min or less, preferably in 1 min or less, more preferably in 30 s or less, still more preferably in 10 s or less, still more preferably in 1 s or less, still more preferably in 0.1 s or less, still more preferably in 0.01 s or less and most preferably in 0.001 s or less.

In a second aspect the invention relates to a method of analysis of the nucleic acid sequence of a nucleic acid analyte comprising the steps: (a) in-situ synthesis of at least one oligonucleotide probe in at least one synthesis region in a miniaturized flow cell; (b) addition of at least one single-stranded or double-stranded nucleic acid analyte to the miniaturized flow cells; (c) ligation or sequence-specific hybridization of the nucleic acid analyte on the oligonucleotide probe; and (d) at least one template-dependent nucleic acid synthesis step, which is accompanied by a change in an optical or electrical signal.

In preferred embodiments of this second aspect the internal volume of the flow cell from step (a) is preferably 40% or less, more preferably 25% or less, still more preferably 10% or less, still more preferably 5% or less, still more preferably 1% or less, still more preferably 0.5% or less, still more preferably 0.1% or less, still more preferably 0.01% or less, still more preferably 0.001% or less and most preferably 0.0001% or less of the volume of the feed line to the fluid reservoir. In preferred embodiments of this second aspect the flow cell from step (a) is characterized in that a fluidic step preferably takes 1 min or less, more preferably 30 s or less, still more preferably 10 s or less, still more preferably 1 s or less, still more preferably 0.1 s or less, still more preferably 0.01 s or less, still more preferably 0.001 s or less and most preferably 0.0001 s or less.

In a third aspect the invention relates to a method of amplification of a target nucleic acid comprising the steps: (a) in-situ synthesis of at least one oligonucleotide probe in at least one synthesis region in a miniaturized flow cell; (b) addition of at least one single-stranded or double-stranded nucleic acid analyte to the miniaturized flow cells; (c) ligation or sequence-specific hybridization of the nucleic acid analyte to the oligonucleotide probe; and (d) at least one cycle of nucleic acid amplification.

In preferred embodiments of this third aspect step (d) comprises the step of a template-dependent nucleic acid synthesis and/or the ligation of an oligonucleotide primer or of an adapter nucleotide to the nucleic acid analyte and/or the step of digestion with a restriction endonuclease. In preferred embodiments of this third aspect, one or more of steps (a) to (d) are accompanied by a change in optical or electrical properties. Preferably this change of an optical property is a change in the localization, the emission, the absorption, or the amount of an optical marker. In preferred embodiments of this third aspect the method additionally comprises the step of an in-situ synthesis of at least one oligonucleotide primer in the miniaturized flow cell, which is secured detachably in its synthesis region. In preferred embodiments of this third aspect the method additionally comprises the release of two or more oligonucleotide primers and their hybridization to form a double-stranded adapter oligonucleotide. It is moreover preferable for the amplification method to be selected from strand-displacement amplification, PCR and rolling-circle amplification. In preferred embodiments of this third aspect the amplification product is released from the surface of the miniaturized flow cell. It is moreover preferable for the amplification product to undergo one or more further processing and/or analysis steps, which are selected from PCR, gel electrophoresis, ligation, restriction digestion, phosphatase treatment, kinase treatment, in-vitro protein translation and in-vivo protein translation.

In a fourth aspect the invention relates to a method of amplification of a target nucleic acid comprising the steps: (a) in-situ synthesis of at least one oligonucleotide probe in at least one synthesis region in a miniaturized flow cell; wherein the oligonucleotide probe has intramolecular hybridization regions; wherein one of the intramolecular hybridization regions is positioned at the 3′ end of the oligonucleotide probe; and wherein a recognition sequence for a nicking endonuclease (I) is present in the hybridization region at the 3′ end of the oligonucleotide probe, or (II) can be generated by a sequence-dependent extension of the hybridization region at the 3′ end of the oligonucleotide probe, or (III) is partially present in the hybridization region at the 3′ end of the oligonucleotide probe and can be completed by a sequence-dependent extension of the hybridization region at the 3′ end of the oligonucleotide probe; (b) sequence-specific hybridization of the intramolecular hybridization regions of the oligonucleotide probe with one another; (c) addition of a DNA polymerase; (d) sequence-dependent synthesis of a complementary DNA strand by the DNA polymerase starting from the 3′ end of the oligonucleotide probe; (e) addition of a nicking endonuclease; (f) production of a recognition-sequence-specific single-strand break by the nicking endonuclease; (g) sequence-dependent synthesis of a new complementary DNA strand by the DNA polymerase starting from the single-strand break produced in (f) with displacement of the previously synthesized complementary DNA strand; and (h) optionally single or multiple repetition of steps (f) and (g); wherein step (c) can take place before, during or after step (b); and wherein step (e) can take place before, during or after one of steps (b), (c) or (d).

In a fifth aspect the invention relates to a method of amplification of a target nucleic acid comprising the steps: (a) in-situ synthesis of at least one oligonucleotide probe in at least one synthesis region in a miniaturized flow cell; (b) addition of a primer molecule; wherein the primer is designed so that, at least at its 3′ end, it has a region that is complementary to the oligonucleotide probe; and wherein a recognition sequence for a nicking endonuclease (I) is present in the region complementary to the oligonucleotide probe of the primer, or (II) can be generated by a sequence-dependent extension of the region complementary to the oligonucleotide probe, or (III) is partially present in the region complementary to the oligonucleotide probe of the primer and can be completed by a sequence-dependent extension of the region complementary to the oligonucleotide probe of the primer; (c) sequence-specific hybridization of the primer molecule to the oligonucleotide probe; (d) addition of a DNA polymerase; (e) sequence-dependent synthesis of a complementary DNA strand by the DNA polymerase starting from the 3′ end of the primer molecule; (f) addition of a nicking endonuclease; (g) production of a recognition-sequence-specific single-strand break by the nicking endonuclease; (h) sequence-dependent synthesis of a new complementary DNA strand by the DNA polymerase starting from the single-strand break produced in (g) with displacement of the previously synthesized complementary DNA strand; and (i) optionally single or multiple repetition of steps (g) and (h); wherein step (d) can take place before, during or after one of the steps (b) or (c); and wherein step (f) can take place before, during or after one of steps (b), (c), (d) or (e).

In a sixth aspect the invention relates to a method of amplification of a target nucleic acid comprising the steps: (a) in-situ synthesis of a plurality of at least one first oligonucleotide probe in at least one synthesis region in a miniaturized flow cell; (b) in-situ synthesis of a plurality of at least one second oligonucleotide probe in at least one synthesis region in a miniaturized flow cell; wherein the distance between any two oligonucleotide probes in each case is selected so that they cannot bind to one another; wherein in each case appropriate first and second oligonucleotide probes are synthesized in the same synthesis region; (c) addition of at least one single-stranded or double-stranded nucleic acid analyte to the miniaturized flow cells; (d) ligation or sequence-specific hybridization of the nucleic acid analyte to a first oligonucleotide probe; (e) addition of a DNA polymerase; (f) sequence-dependent synthesis of a complementary DNA strand by the DNA polymerase starting from the 3′ end of the first oligonucleotide probe; (g) ligation or sequence-specific hybridization of the DNA strand newly synthesized in (f) to a second oligonucleotide probe; (h) sequence-dependent synthesis of a complementary DNA strand by the DNA polymerase starting from the 3′ end of the second oligonucleotide probe; (i) optionally ligation or sequence-specific hybridization of the DNA strand newly synthesized in (h) to a first oligonucleotide probe; and (j) optionally single or multiple repetition of steps (f) to (i); wherein step (e) can take place before, during or after one of steps (b), (c) or (d).

In preferred embodiments of the methods according to the invention for the amplification of a target nucleic acid, the methods contain one or more stringent washing steps, preferably a stringent washing step after step (d) and/or after step (f) of the sixth aspect.

In preferred embodiments of the methods according to the invention for the amplification of a target nucleic acid, the amount of the newly synthesized nucleic acids is determined in real time.

In a seventh aspect the invention relates to a method of production of a support for the determination of nucleic acid analytes by hybridization, comprising the steps: (a) provision of a supporting material and (b) stepwise construction of an array of several different receptors selected from nucleic acids and nucleic acid analogs on the support by spatially-specific and/or time-specific immobilization of receptor building blocks at respective predetermined positions on the or in the supporting material, wherein several different sets of synthesis building blocks are used for the synthesis of the receptors, in order to obtain receptors that are asymmetric, i.e. consisting of several different types of receptor building blocks.

In an eighth aspect the invention relates to a method of production of a support for the determination of nucleic acid analytes by hybridization, comprising the steps: (a) provision of a supporting material and (b) stepwise construction of an array of several different receptors selected from nucleic acids and nucleic acid analogs on the support by spatially-specific and/or time-specific immobilization of receptor building blocks at respective predetermined positions on the or in the supporting material, wherein, in one or more of the predetermined positions the nucleotide sequences of the receptors are selected in such a way that the receptors, in the absence of an analyte specifically bindable thereto, are at least partially in the form of a secondary structure.

In a ninth aspect the invention relates to a method of determination of analytes, comprising the steps: (a) provision of a support with several predetermined regions, on which in each case different receptors, selected from nucleic acids and nucleic acid analogs, are immobilized, wherein in one or more of the predetermined regions the receptors consist of several different types of receptor building blocks, (b) contacting the support with a sample containing analytes and (c) determining the analytes from their binding to the receptors immobilized on the support, wherein the binding of an analyte to a receptor specifically bindable thereto leads to a detectable change in signal.

In a tenth aspect the invention relates to a method of determination of analytes, comprising the steps: (a) provision of a support with several predetermined regions, on which in each case different receptors, selected from nucleic acids and nucleic acid analogs, are immobilized, wherein in one or more of the predetermined regions the receptors, in the absence of an analyte specifically bindable thereto, is at least partially in the form of a secondary structure, (b) contacting the support with a sample containing analytes and (c) determining the analytes from their binding to the receptors immobilized on the support, wherein the binding of an analyte to a receptor that is specifically bindable thereto, comprises the detection of opening of the secondary structure that is present in the absence of the analyte.

In an eleventh aspect the invention relates to a method of determination of analytes, comprising the steps: (a) provision of a support with several predetermined regions, on which in each case different receptors, selected from nucleic acids and nucleic acid analogs, are immobilized; wherein each individual receptor comprises at least one hybridization region, to which an analyte can hybridize specifically; (b) contacting the support with a sample containing analytes; (c) execution of a primer extension reaction; wherein the analyte functions as primer; wherein building blocks carrying one or more signal-emitting groups and/or one or more haptenes, are incorporated in the primer extension reaction; and (d) determination of the analyte from the incorporation of building blocks containing signal groups or haptenes.

In a twelfth aspect the invention relates to a method of determination of analytes, comprising the steps: (a) provision of a support with several predetermined regions, on which in each case different receptors, selected from nucleic acids and nucleic acid analogs, are immobilized; wherein each individual receptor comprises at least one hybridization region, to which an analyte can hybridize specifically; (b) contacting the support with a sample containing analytes; wherein the analytes in the sample were linked, before, during or after the contacting, to one or more signal-emitting groups and/or to one or more haptenes; (c) determination of the analytes by detecting the signal-emitting group(s) or the haptene or haptenes in the analyte.

In a thirteenth aspect the invention relates to a method of amplification of analytes, comprising the steps: (a) provision of a support with several predetermined regions, on which in each case different receptors, selected from nucleic acids and nucleic acid analogs, are immobilized; wherein each individual receptor has, at its 3′ end, a hybridization region to which an analyte can hybridize specifically; (b) contacting the support with a sample containing analytes; and (c) execution of a primer extension reaction; wherein the various receptors function as primers, so that a double-stranded nucleic acid, consisting of analyte and extended receptor, is obtained.

In preferred embodiments of this thirteenth aspect the method additionally contains the following process steps, following on from step (c): (d) thermal denaturation of the double-stranded nucleic acid obtained in step (c); (e) setting of reaction conditions that permit hybridization of analyte and nonextended receptors; (f) execution of a primer extension reaction, with the various nonextended receptors functioning as primers; and (g) optionally repetition of steps (d) to (f).

In preferred embodiments, in primer extension reaction (c) and/or in primer extension reaction (f), building blocks are incorporated that carry one or more signal-emitting groups and/or one or more haptenes.

In further preferred embodiments the method additionally contains the following process step, which is carried out during one of steps (c) to (g) or after one of steps (c) to (g): determination of the analyte from the incorporation of the signal-group-containing and/or haptene-containing building blocks.

In preferred embodiments of the thirteenth aspect, the analyte is an RNA; wherein the various receptors additionally have a region with a primer sequence 1, in the 5′ position to the hybridization region, and wherein the method additionally has the following process steps, which follow on from step (c): (d) ligation of a nucleic acid cassette, which has a region with a primer sequence 2, to the double-stranded nucleic acid obtained in step (c); (e) execution of a two-strand synthesis; (f) execution of at least one cycle of an amplification reaction with addition of a primer with primer sequence 1 and a primer with primer sequence 2.

In preferred embodiments, in step (e) and/or in step (f), building blocks will be incorporated that carry one or more signal-emitting groups and/or one or more haptenes.

In further preferred embodiments the method additionally contains the following process step, which is carried out during one of steps (e) to (f) or after one of steps (e) to (f): determination of the analyte from the incorporation of the signal-group-containing and/or haptene-containing building blocks.

In a fourteenth aspect the invention relates to a method of production of a support for nucleic acid analysis and/or synthesis, comprising the steps: (a) provision of a supporting material and (b) stepwise construction of an array of several different receptors selected from nucleic acids and nucleic acid analogs on the support by spatially specific and/or time-specific immobilization of receptor building blocks at respective predetermined positions on the or in the supporting material, wherein in at least one synthesis region, at least 2 different receptors are synthesized by orthogonal chemical methods.

In a fifteenth aspect the invention relates to a reagent kit, comprising a supporting material and at least two different sets of building blocks for the synthesis of receptors on the supporting material.

In a sixteenth aspect the invention relates to the use of the molecular-biological processing equipment according to the first aspect for the detection and/or for the isolation of nucleic acids; for sequencing; for point mutation analysis; for the analysis of genomes, genome variations, genome instabilities and/or chromosomes; for the typing of pathogens; for genotyping; for gene-expression or transcriptome analysis; for the analysis of cDNA libraries; for the production of substrate-bound cDNA libraries or cRNA libraries; for the production of arrays for the production of synthetic nucleic acids, nucleic acid double strands and/or synthetic genes; for the production of arrays of primers, ultra-longmers, probes for homogeneous assays, molecular beacons and/or hairpin probes; for the production of arrays for the production, optimization and/or development of antisense molecules; for further processing of the analytes or target molecules for logically downstream analysis on the microarray, in a sequencing process, in an amplification process or for analysis in gel electrophoresis; for the production of processed RNA libraries for subsequent steps, selected from: translation in vitro or in vivo or modulation of gene expression by iRNA or RNAi; for the production of sequences that are then cloned by vectors or in plasmids or directly; and/or for the ligation of nucleic acids in vectors or plasmids.

In preferred embodiments of the aforementioned methods and applications, the analyte or nucleic acid analyte or the nucleic acid to be detected and/or to be isolated is selected from the group comprising: a microRNA, a cDNA corresponding to a microRNA, a nucleic acid with a pathogenic action, and a nucleic acid obtained from a pathogen.

A preferred embodiment for the specific detection of nucleic acids or poly-nucleotides is described below. In this embodiment the specificity of hybridization is integrated with the parallel nature of a microarray and the amplification as in a conventional PCR amplification in the method according to the invention.

The miniaturized reaction support used is a microstructure with three-dimensional microcavities, each having at least one inlet and one outlet. Preferably the interior is designed in such a way that it leads as a single long channel from one inlet to one outlet and therefore permits rapid pressure-operated filling with reagents and other media.

This reaction support is first charged by in-situ synthesis e.g. with oligonucleotides, oligonucleotide derivatives or oligonucleotide analogs, which are arranged in rows and columns of separate reaction fields. The individual reaction fields preferably have dimensions of less than 100×100 μm.

Thus, in the reaction support, functional biological molecules are available that can, by specific hybridization, now selectively bind nucleic acids that are contained in a sample that is added. These target molecules are accordingly bound depending on the sequences that were produced previously during the in-situ synthesis. In the next step, all nucleic acids that were not bound to the desired extent are washed away. Only the specifically bound nucleic acids still remain. However, the amount of these bound nucleic acids may be below the limit of detection of a confocal, e.g. based on a scanning laser, or parallel, e.g. based on CCD chips, optical detector according to the prior art.

The bound material is now submitted to nonspecific or specific enzymatic amplification, which is not directed at additional specific primers. Numerous methods for this are known by a person skilled in the art. For amplification by PCR, a cassette containing the necessary primer sequences can be ligated to the bound nucleic acids. For this it may be useful to treat the sample first with one or more restriction nucleases, so that the specific sequences known by a person skilled in the art form at the cleavage sites. Alternatively it is possible to employ commercial kits, such as the “GenomePlex Whole Genome Amplification WGA Kit”, available from Rubicon Genomics, USA, or from Sigma-Aldrich, USA.

Following the amplification step, the nucleic acid material is again hybridized to the oligonucleotides of the array. It may be useful to carry out other intermediate steps, such as a heating step for separating the strands. What is important in all steps is that, in each case before a washing step or after a processing step, there is the opportunity for those target molecules that are to be processed further to bind to the matrix of functional biological molecules, thus in this embodiment to the microarray of oligonucleotides. After the hybridization step, washing is carried out again, so that all nonspecific material is removed completely or partially.

Detection can now be carried out; this can be performed as described above, with various methods and devices for the use of microarrays known by a person skilled in the art. Examples that may be mentioned are microscopes, optical scanners, laser scanners, confocal scanners, or parallel, e.g. CCD-chip-based, optical detectors, which record more than one measurement point at a time, or can even record the whole reaction support in its entirety, and mixed forms of the devices described above, e.g. scanners with CCD-lines. Examples of signals that can be used in the analysis of the reaction results on the reaction support or array include the following signals that are well known in the industry:

Optical signals
Fluorescence (organic and inorganic fluorophors, fluorescent biomolecules),
Light scattering (e.g. gold particles in nm-dimensions),
Chemiluminescence,
Bioluminescence;

Electrical signals
Flow of current,
Redox reactions.

Alternatively, for detection, first by repeating the washing-separation steps, further enrichment of the material relevant to the result can be achieved, and moreover the signal-noise ratio can be improved.

An important technical feature of the equipment required for this is suitable changing of fluids or reagents. In particular, equipment with the possibility of changing of the fluids or reagents that is rapid and can be automated is therefore an object of this invention. Such equipment is described in WO 00/13017 and in WO 00/13018, to which reference is hereby made.

### 6 PREFERRED EMBODIMENTS

The present invention will now be described more precisely. Various aspects, features and embodiments of the invention are described in greater detail in the following sections. Every aspect, feature, and embodiment thus defined can be combined with any other aspect, feature or embodiment, unless expressly stated otherwise. This also includes multiple combinations of aspects, features or embodiments. In particular, any preferred feature or any preferred embodiment can be combined with one or more preferred features or preferred embodiments.

### 6.1 Primers in DNA Processor

Oligonucleotides, oligonucleotide derivatives or oligonucleotide analogs are synthesized in the reaction support in such a way that their 3′-OH end can be extended with a polymerase. This can be effected e.g. by linkage of the 5′-end to the reaction support, with the 3′-OH end remaining free. Nucleic acids that are to be analyzed and can be copied by a polymerase are hybridized to the anchored molecules and the 3′-OH end of the probes is extended by the polymerase through linkage of nucleotides or nucleotide analogs. During extension, a copy of the hybridized molecule can be made, but does not have to be made. In particular, the newly formed strand can belong to another class of compounds than the hybridized strand, thus, for example, nucleic acid derivatives and analogs can be inserted into the strand or can be linked on. The course of the reaction can optionally be monitored optically, e.g. by the incorporation of modified nucleotides or the presence of additional signal-emitting substances, which for example interact with DNA. Alternatively the hybridized molecule, not produced in the reaction support, can function as primer and can be extended. Once again, a copy of the molecule produced in the reaction support can be made, but does not have to be made. An example of extension of the molecule functioning as primer, without making a copy of the hybridized strand, are template-independent extension reactions such as are known by a person skilled in the art. For example, this can be the production of poly(A) tails, which are formed by some polymerases.

### 6.2 Integrated Sample Preparation

Methods for the processing or preparation of an analytical method can also be carried out directly in the reaction support. These include for example the removal or conversion of interfering accompanying substances (for instance by enzymatic processing), attachment of signal-emitting groups or their precursor stages and attachment of certain groups for the binding of ligands such as proteins, nucleic acids, signal-emitting molecules or their precursor stages. Said attachment can take place by chemical or for example also enzymatic methods that are known by a person skilled in the art. The reaction support can moreover be used for the purification of sample molecules that is based on the affinity of the desired sample molecules from the biological sample mixture for probe molecules located on the surface of the reaction support. This method, which is similar to affinity chromatography, is based on the binding of the sample molecules to said probe molecules and one or more washing steps, in which the temperature can also be varied.

6.3 Method of Solid-Phase Production of Full-Length cDNA Libraries

For this, “capture oligos” are synthesized in the reaction support in such a way that, with their sequence, they are specific for all or a selection of genes, to a region downstream of the poly(A) tail. It may be advantageous to select this region near the 5′ end. In this way, transcripts are extracted specifically, on a solid-phase support, from an mRNA preparation or an mRNA population that has already undergone further processing (e.g. a cDNA library).

As the next step, in the synthesis of capture oligos with distal 3′ end, complementary strands to the isolated strand can be synthesized. This is effected by adding appropriate enzymes and other feed materials known by a person skilled in the art.

In a further step, copies of the strand linked covalently to the solid phase can now be made. All full-length sequences (starting from the binding site of the capture oligos) have by definition a poly(T) segment at the distal end of the strand. This can be used for linear amplification with corresponding poly(A) primers. An advantage of said linear amplification is little distortion of the concentration ratios of individual transcripts to one another. Alternatively, in the capture oligo, conservative primer sequences can also be inserted proximally to the support, permitting exponential amplification of the isolated strands.

6.4 Combination of Solid-Phase cDNA Libraries with Analysis on an Array

In another embodiment, the isolation and amplification of transcripts or of genomic or other sequences, as described above, are combined with analysis on a polymer probe array synthesized in situ, so that either both types of oligo (capture oligo and analysis oligo) are accommodated in a common support or in compartments of the support that are connected to one another automatically.

In one example, with 35-mer capture oligos, at relatively high stringency or temperature, target molecules can be isolated and can be amplified as described above. In the same reaction support, far shorter analysis oligos with length of e.g. 20 nucleotides were also synthesized beforehand. It can be seen by a person skilled in the art that because of the difference in length of the oligos on the basis of stringency or temperature, serial execution of the process steps of isolation, amplification and analysis can be carried out.

Compartments that contain individual process steps sequentially can be created by means of hydrophobic barriers, valves, separate reaction chambers or similar technical details of the reaction support, which are known from microreactor technology.

### 6.5 “Sequencing by Synthesis” in the Processing Equipment According to the Invention

The method according to the invention can, in another embodiment, be used for carrying out “sequencing by synthesis”. First, a microarray of oligonucleotides, oligonucleotide derivatives or oligonucleotide analogs (probes) is prepared in the reaction support and is hybridized to a nucleic acid sample to be analyzed. The molecules produced in the microarray contain free 3′-OH ends, so that—as is known by a person skilled in the art—extension of the ends by a polymerase will be possible. Several methods are known that permit attachment of just one nucleotide and joining of the phosphate backbone, as the nucleotides still contain a blocking group. This blocking group can be split off inside the miniaturized reaction support, so that a polymerase-extendable nucleotide is formed. For detection, the nucleotide can contain e.g. signal-emitting groups or precursors thereof, which can also be split off inside the miniaturized reaction support (for instance fluorophors). Alternatively, the cleavable blocking group can be bound to a ligand that is linked to a signal-emitting group or a precursor thereof (e.g. fluorescence-labeled antibody). With cycles of nucleotide addition, optionally ligand binding, detection, splitting-off of the blocking group (and optionally of the signal-emitting group) and further nucleotide addition, it is thus possible to elucidate sequences of bound nucleic acid molecules that are to be analyzed.

The processing equipment according to the invention offers considerable advantages for this technology in comparison with the testing formats known by a person skilled in the art.

For example, in the test systems developed by 454 Life Sciences, Helicos or Solexa, which were described in more detail in section 2.3 (Bennett S T, Barnes C, Cox A, Davies L, Brown C. Pharmacogenomics. 2005 June; 6(4):373-82. Warren R L, Sutton G G, Jones S J, Holt R A. Bioinformatics. 2006 Dec. 8; [Epub ahead of print]. Bentley D R. Curr Opin Genet Dev. 2006 December; 16(6):545-52. Bennett S. Pharmacogenomics. 2004 June; 5(4):433-8. Margulies, M. Eghold, M. Etal. Nature. 2005 Sep. 15; 437(7057):326-7. Patrick Ng, Jack J. S. Tan, Hong Sain Ooi, Yen Ling Lee, Kuo Ping Chiu, Melissa J. Fullwood, Kandhadayar G. Srinivasan, Clotilde Perbost, Lei Du, Wing-Kin Sung, Chia-Lin Wei and Yijun Ruan Nucleic Acids Research, 2006, Vol. 34, No. 12. Robert Pinard, Alex de Winter, Gary J Sarkis, Mark B Gerstein, Karrie R Tartaro, Ramona N Plant, Michael Egholm, Jonathan M Rothberg, and John H Leamon BMC Genomics 2006, 7:216. John H. Leamon, Michael S. Braverman and Jonathan M. Rothberg, Gene Therapy and Regulation, Vol. 3, No. 1 (2007) 15-31), the gene segments to be investigated without information about their identity are immobilized on surfaces so that they can then be sequenced by the method described. The information about longer gene segments is then obtained by assembly of the small individual data bioinformatically. This means that always the complete genome must be analyzed and the number and length of the individual sequenced regions must exceed a critical size, if assembly is to be made possible at all, by sufficient overlapping of the segments. In many cases, however, we are only interested in the sequence of a part of the genome. In the processing equipment according to the invention it is possible for desired gene segments to be specially selected for sequencing through sequence-specific immobilization (hybridization of the desired segment to a probe specific thereto, synthesized in the reaction support of the processing equipment according to the invention). Thus, by choosing the number and sequence of the probes synthesized and prepared in the reaction support of the processing equipment according to the invention, the number and identity of the desired gene segments of the sample can be established. There is no limitation as to the number, nature or minimum length of the sequenced segments, as no subsequent bioinformatic assembly has to be carried out.

In this preferred embodiment, the processing equipment according to the invention can in particular be used for multistep processing and analysis of sample material in the following way: by providing probes in the reaction support of the processing equipment according to the invention that are specific to gene segments to be analyzed, first it is possible to select desired gene segments by binding to the probes. A washing step can optionally take place, to remove undesirable sample material from the reaction support. Amplification of the sample material can then take place, and this can already provide information about the sequence of the bound. Numerous methods for this are known by a person skilled in the art. Then, optionally, sequencing of the bound and optionally amplified sample molecules can take place by the method described. This sequential processing and analysis of sample material is greatly simplified by the design of the reaction support as microfluidic unit and therefore offers a fundamental improvement over the prior art.

6.6 Amplification of the Signal Instead of the Target Molecule in One of the Steps after the First Initial Binding

Examples of said signal amplification are known by a person skilled in the art, and include, among others, Rolling Circle amplification, tyramide-mediated amplification, chemiluminescence and bioluminescence, phosphatase-induced amplification or the decoration of the bound target molecules with one or more further oligonucleotides, which for their part have already been labeled, e.g. when using “branched DNA” or “bDNA” from the company Genospectra, USA (Collins M. L. et al.; Nucleic Acids Res. 25(15); 2979-2984; 1997). Conjugates of streptavidin and an oligonucleotide linked to it via the 5′ end can preferably be used. This can bind to biotin units previously applied on the sample molecule or on probe molecules that bind to the sample molecule. Then after adding a circular nucleic acid, a rolling-circle amplification known by a person skilled in the art can take place, using the oligonucleotides bound to the streptavidin. The use of a process step (after the last binding deemed sufficient or in between) that permits amplification of the measured signal instead of a further amplification of the target molecule, can have a favorable effect on the costs of the assay. Another possible advantage is minimal distortion of the ratio of the target molecules in the sample.

### 6.7 Reaction Supports

Several reaction supports can be used for most of the embodiments of the methods and molecular-biological processing equipment according to the invention. What is important is the targeted feed of reagents or fluids and the corresponding provision of functional biological molecules by positionally resolved and/or time-resolved immobilization. The reaction supports can in principle be flat glass plates, such as are used as microscope slides and for microarrays, where the surfaces can be prepared with one of the numerous configurations known by a person skilled in the art for the binding of molecules, for example with reactive or activatable functional groups (epoxy groups, amino groups etc.). Alternatively the reaction supports can be coated with another layer, e.g. a gel, a polyacrylamide or a porous coating, which can also increase the loading capacity of the reaction support.

The reaction supports can be in the form of three-dimensional microstructures, as described for example in WO 00/13018, in WO 02/46091 and in WO 01/08799. According to these, the reaction supports can contain a large number of small holes or pores, which can be arranged parallel or orthogonal to feed lines and discharge lines. Alternatively it may be useful to use a support that immobilizes a set of beads, microspheres or microparticles physically, electrostatically, fluidically or chemically, as described e.g. in WO 02/32567 or known from the company Illumina, USA.

Apart from glass, many other organic and inorganic materials are known for the reaction supports, for example silicon, plastics, polypropylene, resins, polycarbonate, cyclic olefin copolymers or mixtures of these materials.

Three-dimensional structures can be integrated directly in the equipment according to the invention with suitable connecting techniques. Flat or unenclosed reaction supports are accommodated correspondingly in a flow cell or some other three-dimensional reaction space, so that the necessary exchange of reagents or fluids can take place. These constructions can be permanent, so that for normal operation no changing of the actual flat or unenclosed reaction support is envisaged. This can be effected by gluing, screwing, indirect holding, clipping or clamping. Reversible fitting of the reaction support in the three-dimensional reaction space can also be provided. Methods of holding reaction supports in flow cells and measuring devices are known by a person skilled in the art.

The three-dimensional reaction spaces or closed structures are then provided with corresponding connections for supply with fluids and reagents.

### 6.8 Oligos are Copied Enzymatically

The molecules produced in the reaction support can function as a template and are copied. This can be utilized not only for analysis of the reaction support, if signal-emitting building blocks are incorporated during copying, but can be used to produce a copy of the reaction support in the form of a mixture of soluble copies of the molecules synthesized in the reaction support. After that, the reaction support can be reused, e.g. for copying again. An example of such a process is the copying of DNA molecules in the reaction support by a primer extension reaction by means of a polymerase. During this, by using a thermostable polymerase, an amplification can also be carried out, if for example by means of an excess of primer and suitable changes in temperature during the reaction, repeated binding and extension of the primers is carried out. The resultant copies can then be isolated from the reaction support by washing. A primer extension reaction can also be used without washing, e.g. to convert DNA single strands synthesized in the reaction support to double strands. These can be used for the analysis e.g. of proteins that bind or modify double-stranded DNA.

During the operation of copying of the molecules of the reaction support, another type of molecule may also form. For example, DNA synthesized in the reaction support can be transcribed to RNA. This can for example be effected by the prior conversion of the DNA single strands synthesized in the reaction support to double strands, as described, and subsequent transcription. Numerous methods for this are known by a person skilled in the art. It is moreover possible to incorporate or attach nucleic acid analogs or derivatives, which are not natural DNA or RNA building blocks.

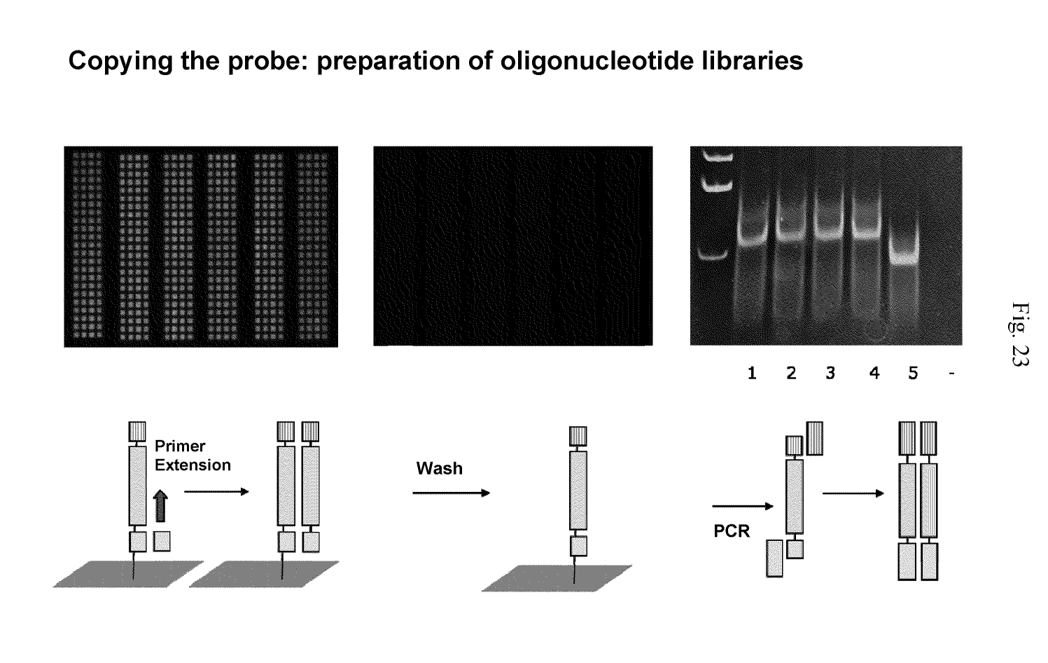

FIG. 23 illustrates the embodiment described and presents data from experiments that provide evidence of successful copying of probe molecules synthesized on the surface of the reaction support in the manner of primer extension. The copies prepared can then be detached from the reaction support by washing, and successfully used as the template in a PCR reaction, so that they are amplified.

### 6.9 Configurations of the Molecules Synthesized in the Reaction Support

The molecules synthesized in the reaction support can belong to various classes of compounds. For example, DNA or RNA molecules, even peptides, can be synthesized in the reaction support. Furthermore, it is possible to synthesize various derivatives and/or analogs of these classes of compounds in the reaction support. These include peptide nucleic acids (PNA), locked nucleic acids (LNA), various nucleobase-modified nucleic acid derivatives and analogs, e.g. nucleic acids with altered hybridization behavior or attached functional groups such as haptenes, fluorescent dyes, luminescent groups or precursors thereof, photoreactive groups, inorganic particles, photoisomerizable groups or groups with a particular desired binding or reaction behavior or a desired optical behavior. These include among others but not exclusively gold nanoparticles, stilbenes, azobenzenes, nitrobenzyl compounds, biotin, digoxigenin, quantum dots, phosphate, phosphorus thioate, groups that increase the resistance of the molecule e.g. to enzymes, groups that are substrates for enzymes, etc.

It is also possible for mixtures of molecules to be produced in the reaction support. The molecules can also contain branchings or dendritic structures. It is moreover possible to synthesize molecules in the reaction support that belong to several classes of compounds or consist of various linked parts, which in each case belong to different classes of compounds. Linkage can take place directly or via particular linker groups. For example, nucleic acids can be linked to peptides and/or proteins. Generally, for production and modification of the desired molecules it is possible to use not only e.g. organic-chemical methods, but also e.g. enzymatic methods.

### 6.10 Production of Various Reagents for the New Molecular-Biological Method In the Same Reaction Support (Specific Primers or Other Functional Oligonucleotide Probes)

The specific primers, aptamers, ribozymes, aptazymes or other oligonucleotide probes or functional oligonucleotides or polynucleotides can be produced in the same reaction support and in several embodiments also on the same array and can be dissolved in one of the process steps. For this, they can either be provided with suitable labile linkers or can be produced as copies of oligonucleotide probes produced on the reaction support. By using known methods of production of such arrays from nucleic acid polymers, e.g. in the form of a so-called microarray, it is possible to produce very many (typically more than 10) different nucleic acid polymers with length of at least more than 2, typically more than 10 bases.

In one embodiment, a portion of the microarray or of the nucleic acids that were immobilized thereon is provided as copyable matrixes for enzyme-based synthesis by a copying operation. After their actual synthesis, they are available in a copyable state and can be amplified in an enzyme-based method by adding appropriate reagents and auxiliary substances, such as nucleotides.

The next step in the method according to the invention now consists of copying the molecules synthesized on the solid phase by means of appropriate enzymes. For this, numerous enzyme systems are known and commercially available. Examples are DNA polymerases, thermostable DNA polymerases, reverse transcriptases and RNA polymerases.

The reaction products are characterized by great diversity of the sequence that can be programmed at will, indirectly via the matrix molecules during the preceding synthesis operation. A microarray from Geniom-Instrument can synthesize, in a micro-channel as the reaction space, 6000 freely selectable oligonucleotides with a sequence of up to 30 nucleotides in a microarray arrangement. After the copying step there are correspondingly up to 6000 freely programmable DNA-30-mers or RNA-30-mers in solution that can be made available as reactants for a subsequent process step.

For the start of the copying step, in some embodiments it will be necessary to add so-called primer molecules, which serve as the initiation point for polymerases. These primers can consist of DNA, RNA, a hybrid of the two, or modified bases. The use of nucleic acid analogs, for example PNA or LNA molecules, is envisaged in some embodiments. For the creation of a recognition site for the primer, it may be desirable to add, at the end of each nucleic acid polymer on the support, a uniform sequence, either as part of the synthesis or in an additional step by means of an enzymatic reaction, such as ligation of a previously prepared nucleic acid cassette. In one variant the distal end of the sequence synthesized on the support is self-complementary and can thus form a hybrid double strand, which is recognized as the initiation point by the polymerases.

Examples of embodiments of the method according to the invention and of process steps using said nucleic acid polymers freely present in solution are:
the production of primers for primer-extension methods, strand-displacement amplification, polymerase chain reaction, site directed mutagenesis or rolling circle amplification,
further processing of the analytes or target molecules for the logically downstream analysis on the microarray, in a sequencing process, in an amplification process (strand displacement amplification, polymerase chain reaction or rolling circle amplification) or for analysis in gel electrophoresis,
production of processed RNA libraries for subsequent steps, such as translation in vitro or in vivo or the modulation of gene expression by iRNA or RNAi,
production of sequences that are then clonable by vectors or in plasmids or directly,
ligation of the nucleic acids in vectors or plasmids.

The use of nucleic acids as hybridizable reagent is common to all of these methods. Furthermore, there are also methods in which nucleic acid polymers are not used, or are not used exclusively, via a hybridization reaction. These include aptamers, ribozymes and aptazymes.

Production of the nucleic acid polymers for the method according to the invention via a copying reaction offers, as an additional advantage at several points of the method, the possibility of introducing modifications or markers in the reaction products by known methods. These include labeled nucleotides, which are modified e.g. with haptenes or optical markers, such as fluorophors and luminescence markers, labeled primers or nucleic acid analogs with special properties, for example special melting point or accessibility for enzymes.

Initiation on the matrix nucleic acids can in principle be carried out by all methods that are known by a person skilled in the art for the initiation of an enzymatic copying operation of nucleic acids, thus for example from the applications polymerase chain reaction, strand displacement and strand displacement amplification, in-vitro replication, transcription, reverse transcription or viral transcription (representatives of this are T7, T3 and SP6).

In one embodiment a T7, T3 or a SP6 promoter is inserted in a part or all nucleic acid polymers on the reaction support.

According to another embodiment, nucleic acid molecules are synthesized in the reaction support, and serve for the binding of microRNAs. The nucleic acid molecules can consist of DNA, but also of nucleic acid analogs that have a modified hybridization behavior.

According to another embodiment, nucleic acids that are bound to the molecules synthesized in the reaction support are linked by enzymatic methods to a universal group. This can be effected by extension by template-independent polymerases, e.g. poly(A) polymerase or telomerase.

In another embodiment a primer extension reaction is used for generating double strands from single-stranded nucleic acid molecules synthesized in the reaction support. These can serve for the analysis of binding or modification events by means of e.g. proteins that bind to the double strand. For this, it may be desirable to incorporate general sequence segments in the molecules synthesized in the reaction support, which for example serve as the binding site for one or more primers. In addition, chemical groups can be inserted that make covalent linkage of the two strands possible. Numerous examples of this, e.g. the use of psoralen, are known by a person skilled in the art.

In another embodiment, the proportion of the array of nucleic acids that is provided for these reaction products serves for the initiation of an isothermal copying reaction. The strand-displacement reaction is a representative of these methods. Numerous variants of this are known by a person skilled in the art. For example, a primer is selected that binds to the matrix polymers at their distal end and can then be extended there in the 3′ direction. All or a certain proportion of the nucleic acid polymers on the support contain this primer sequence distally. Next, an enzyme is added, for which the primer contains a recognition site, so that a single-strand break is induced. The usual procedure envisages for this the use of a restriction nuclease, e.g. N.BstNB I (obtainable e.g. from the company New England Biolabs), which by its nature only introduces single-strand breaks (so-called nicks), as it cannot form dimers.

According to another embodiment of the present invention, double-stranded, circular nucleic acid fragments are prepared, wherein one strand is anchored on the surface of the support and the other strand comprises a self-priming 3′ end, so that elongation of the 3′ end can take place. The enzymatic synthesis comprises, in this variant of the method according to the invention, a replication analogous to the known rolling-circle mechanism for the replication of bacteriophages, wherein one strand of the circular nucleic acid fragments is anchored on the surface of the support and can be copied many times. If at first a double-stranded closed nucleic acid fragment is present, the second strand can first be opened by a single-strand break, with formation of a 3′ end, starting from which the elongation takes place. Splitting-off of the elongated strand can take place enzymatically, for example. By adding nucleotide building blocks and a suitable enzyme, synthesis of the partial sequences that are complementary in each case to the nucleic acid strands that are anchored to the base sequences on the surface of the support then takes place.

According to another embodiment, single-stranded, circular DNA molecules are used for copying in a rolling-circle amplification. The primer used for this can be nucleic acid molecules synthesized in the reaction support, or nucleic acid molecules that hybridize to the molecules synthesized in the reaction support. Preferably, oligonucleotides linked to streptavidin can also be used as primer. The streptavidin-biotin conjugate can have been bound to biotin units beforehand, which were previously linked to hybrids of probe molecules and sample molecules. These nucleic acid molecules that hybridize to the molecules synthesized in the reaction support can contain a universal group, for instance a poly(A) tail. For this method, it may be desirable to use universal binding sites in the circular DNA molecule for the binding of the primer. There is then formation of long concatemers, into which signal-emitting molecules are incorporated and which can be employed for the analysis.

According to another embodiment, the resultant concatemers can serve as template for further extendable molecules. These hybridize to the strand formed by rolling-circle amplification and are extended by a polymerase. As several molecules can bind one after another, as a result of its extension a molecule can reach a length at which it adjoins the end of a molecule bound to the same strand. In this case extension can continue if the hybridization of the second molecule is displaced by the continuing extension (“strand displacement”) and the hybridization region of the second molecule is copied again by the extension of the first molecule. Through dissociation of the hybridization of a molecule, single-strand regions form once again, which can serve as template for an extendable molecule. This results in the formation of complex, branched dendritic structures. During the extension of the bound molecules, in particular signal-emitting groups or precursor stages thereof or haptenes can also be incorporated in the growing strand. Molecules that bind to the extended strands can also contain signal-emitting groups or precursor stages thereof or haptenes. The structures formed can also be bound by substances which, through binding to the structures formed by extension, experience a change in one or more of their optical properties.

The products of the copying operation can in various ways acquire labels, binding sites or markers that are required for further processing or for use in further assays or processes.

These include markers and labels that permit direct detection of the copies and are known by a person skilled in the art from other methods of copying nucleic acids. Fluorophors are an example of this. Furthermore, binding sites can be provided for methods of indirect detection or for purification processes. Examples include haptenes, such as biotin or digoxigenin.

The labels, binding sites or markers can, in one variant, be introduced by modified nucleotides. Another route is possible when using primers for the initiation of the copying operation. The primers can be introduced into the reaction already with label, binding sites or marker.

Labels, binding sites or markers can be introduced subsequently, by treating the reaction products of a subsequent marking reaction with generic agents that react with the nucleic acids. Examples are cisplatin reagents or nanogold particles, as are available for example from the company Aurogen, USA. As an alternative, labels, binding sites or markers can also be introduced by means of a further enzymatic reaction, for example catalyzed by a terminal transferase.

In a preferred embodiment the copies of the matrix nucleic acids are in their turn used for reaction with the bound target nucleic acids. Initiation of their synthesis as copying products of nucleic acid probes can take place during or after the specific binding of the target molecules. In an especially preferred embodiment, first the nonspecifically bound or unbound sample material is washed away. The sequences of the nucleic acid probes to be copied are selected so that the sequence that is to be analyzed later in a hybridization reaction does not form until there is successful extension of the individual copied, dissolved nucleic acid polymers. These segments can then in their turn be detected by another region of the array.

In a variant, for production of the signal it can be envisaged that for initiation of the copying operation the primers already bear a modification that assists the production of the signal. An example of such a modification is a primer that carries a branched-DNA structure in its 5′-segment in a region that is not required for the hybridization to the matrix (regarding bDNA, see above).

Another variant envisages that two primers with oppositely directed specificity are prepared for each target sequence, e.g. an individual gene or exon, so that efficient exponential amplification takes place in a PCR or isothermal amplification.

With simultaneous reaction of copying operation, amplification and hybridization to the analytical probes, the complete analysis of a mixture of target nucleic acids can be carried out are in a very compact and simplified format. Said complete analysis can for example elucidate the detection of all expressed genes—without prior sample amplification and with very simple sample preparation.

An associated device, as a preferred embodiment of the equipment according to the invention, consists of
a) equipment for the in-situ synthesis of the arrays of matrix polymers and analytical nucleic acid probes,
b) elements for the execution of fluidic steps, such as sample addition, addition of reagents, washing steps and/or sample withdrawal
c) a detection unit for detecting an optical or electrical signal,
d) a programmable unit for controlling the synthesis,
e) a programmable unit for controlling the fluidics, the detection and the storage and management of the measurement data.

In a further embodiment the extended polymers are brought into contact with analytical nucleic acid probes, which can be used again for extension in the form of a primer extension. The setup of a primer extension experiment is known from the technical literature. The signal of the primer extension to these analysis probes is then evaluated to determine the result of analysis. Said analysis can be, for example, determination of single nucleotide polymorphisms (SNPs) in genomic DNA. For this, extendable primers are first copied on matrix nucleic acids. The sequence is selected so that the SNPs to be investigated are localized in the 3′ region after the primer sequence on the target nucleic acid. In the next step, these primers are extended beyond the sequence of the SNPs to be detected. Then the reaction products of this elongation are investigated by primer extension or directly by hybridization and the results are recorded for determination of the SNPs queried in the analysis. In the programmable device, for the user of the device according to the invention the data are presented in such a way that, for example, the user receives directly a report with the positions of the bases and the bases found.

The great advantage of the invention is that for these genotyping or SNP-analysis assays, still only a universal, generic sample preparation is necessary. Primers and reagents that are specific to individual genotypes or SNPs are not required, as all sequence specificity arises from the in-situ synthesis of the underlying matrix arrays and the analysis array. In the embodiment in which these two are combined in one reaction support, the genotyping and SNP analysis is accordingly maximally simplified.

6.11 Production of Synthetic Genes and Other Synthetic Nucleic-Acid Double Strands Using the Method According to the Invention by Processing Nucleic Acids that were Produced Outside of the Reaction Support

For this, high-quality nucleic acids freely programmable in the sequence are prepared in the form of oligonucleotides, in order to produce synthetic coding double-stranded DNA (synthetic genes). The method according to the invention is used for this.

The oligonucleotides serving as building blocks of the synthetic gene are produced by synthesis in the reaction support. The use of support-bound libraries of nucleic acid probes for the synthesis of synthetic genes is described in PCT/EP00/01356. The synthesis of oligonucleotides by copying support-bound nucleic acids e.g. for gene synthesis or for the production of reagents such as siRNAs or aptamers is described in DE 103 53 887.9. In both methods, oligonucleotides with a freely selectable sequence in a range of 10-100, optionally even up to 500 nucleotides, are prepared for the subsequent processes, such as the construction of synthetic genes. Furthermore, oligonucleotides that were produced outside of the reaction support can be linked to the oligonucleotides synthesized in the reaction support by methods known by a person skilled in the art.

In one embodiment of the method according to the invention, further process steps, which comprise the utilization, purification, modification or refinement of the oligonucleotides or the partial or complete construction of the target sequence, thus optionally of the finished synthetic gene, are carried out according to the method in a corresponding reaction support.

6.12 Production of Synthetic Genes and Other Synthetic Nucleic-Acid Double Strands Using the Method According to the Invention by Processing Nucleic Acids that were Produced Directly in the Reaction Support or in The Microarray
a. synthesis and detachment by labile linkers
b. synthesis via copying of nucleic acid probes

In one embodiment, high-quality nucleic acids freely programmable in the sequence are prepared in the form of oligonucleotides, in order to produce synthetic coding double-stranded DNA (synthetic genes). The method according to the invention is used for this.

The oligonucleotides serving as building blocks of the synthetic genes are produced by synthesis and detachment by means of a labile linker or by synthesis via copying of nucleic acid probes. The use of support-bound libraries of nucleic acid probes for the synthesis of synthetic genes is described in PCT/EP00/01356. The synthesis of oligonucleotides by copying of support-bound nucleic acids e.g. for gene synthesis or for the production of reagents such as siRNAs or aptamers is described in DE 103 53 887.9. In both methods, oligonucleotides with freely selectable sequence in a range of 10-100, optionally even up to 500 nucleotides, are prepared for the subsequent processes, such as the construction of synthetic genes.

In one embodiment of the method according to the invention, further process steps, which comprise the utilization, purification, modification or refinement of the oligonucleotides or the partial or complete construction of the target sequence, thus optionally of the finished synthetic gene, are carried out according to the method in a corresponding reaction support.

6.13 Ligation Mediated by Probes from Arrays

In one embodiment, two strands are linked, one of which is a probe molecule synthesized in the reaction support. Linkage is made possible by a template strand, which brings the two strands to be linked in close proximity. Alternatively the probe molecule synthesized in the reaction support can serve as template, which brings two further strands in close proximity and thus makes linkage possible. In this process, ligation can for example by catalyzed by a ligase, but can also take place by chemical coupling reactions known by a person skilled in the art. In all the cases mentioned, the probe molecule synthesized in the reaction support can either be immobilized on the surface of the reaction support or can have been detached prior to linkage. Alternatively, copying of the molecules of the reaction support can also take place prior to linkage and linkage with the molecules represented by the copy can take place. For the copying, it is possible to use methods known by a person skilled in the art, for instance a primer extension reaction.

### 6.14 Lab on a Chip

The special design of the reaction support as microfluidic system in combination with pumping systems is ideally suitable for sequentially executing modifications of molecules produced on the reaction support or of molecules that bind to the molecules produced on the reaction support. As the molecules to be modified are immobilized on the reaction support or bind to it, washing steps between the various modification events are greatly simplified relative to current methods. A great many molecular-biological processes known by a person skilled in the art contain several successive individual steps of particular modifications, between which a purification step takes place. These comprise e.g. enzymatic modifications such as amplification, primer extension, ligation, phosphorylation or dephosphorylation, nuclease treatments etc. The purification steps are for example binding and washing of the sample using affinity columns, precipitation steps, gel electrophoresis methods etc.

In one embodiment the invention is employed for carrying out successive modification events of molecules. In this case the design of the reaction support, whose microfluidic channels can be irrigated with solutions and mixtures, provides a considerable simplification of such processes in comparison with methods of the prior art. Optionally, the individual modification steps can in each case be followed by a washing step, for example to remove substances of a modification step that might interfere with a subsequent step.

### 6.15 Improved Polymer Probe Arrays

In one embodiment, in particular asymmetric polymer probes are used for the polymer probes. These make it possible to carry out the invention in a variant in which such probes, which represent full-length products, are thermodynamically preferred owing to other factors, rather than merely from the fact that they are full-length products. This is achieved in that the probes contain individual building blocks with an especially strong binding behavior. These special building blocks are inserted asymmetrically or in a later step during the polymer synthesis. This leads to an asymmetry that endows the probes with thermodynamic properties which influence the binding behavior.

In the case of nucleic acids, analogs are employed that contribute to stronger binding to the complementary bases. Alternatively, distally to the probe-molecules, building blocks are inserted that influence the binding behavior and contribute to stronger binding of these probes. Peptide derivatives are an example of this. For example “Minor Groove Binders”, which are also used in the polymerase chain reaction, are known by a person skilled in the art.

Various methods are available for the in-situ synthesis of polymer probe arrays on a support. They have the common purpose of providing an elegant route for the production of these arrays that conserves resources and is economical, and generally provides an especially well defined substrate for the subsequent analyses. Moreover, by means of in-situ methods it is possible to produce arrays with an especially large number of different receptor probes on a reaction support.

A substantial disadvantage of such methods is, however, that in the case of an in-situ synthesis process the product of the synthesis cannot then be purified. To avoid this disadvantage, in so-called “off-chip” syntheses of polymer probes the corresponding DNA molecules are produced using conventional methods and then are purified in such a way that almost exclusively the full-length product is available for the synthesis. Only this population of molecules is then arranged as an array on the substrate. This method has the drawback, however, that arrangement of the prepared polymer probes on the array is very expensive.

Deficient purification can, in conjunction with the specific yield of the in-situ synthesis, lead to marked losses in terms of quality of analysis, if the proportion of full-length product is comparatively low. This is particularly important in the case of DNA microarrays, as the length of an immobilized DNA probe molecule determines the specificity of the potential hybridization reaction with a sample molecule. This specificity is in its turn a determining parameter for the analytical potential of a DNA microarray.

In existing methods for the in-situ production of polymer probe arrays, all successive addition steps are carried out with individual building blocks of these probes. None of these steps has a coupling rate of 100%. It is evident to a person skilled in the art that a chemical sequential condensation, such as in the case of an in-situ synthesis of polymer probes, cannot result in 100% full-length product. In DNA synthesis, the coupling rates for the conventional column techniques, after many years of optimization and using the most efficient known chemical method (phosphoroamidite method according to Caruthers), are still below 100%.

With comparatively low yields in the sequential attachment of synthetic building blocks, the proportion of full-length product can, after a certain number of synthesis cycles, fall below a critical value, so that the analysis result is certainly not characterized by this full-length product. In the photolithographic in-situ synthesis of DNA with MeNPOC protective groups it has been stated, for example, that the coupling rate of the individual addition steps is below 95% (Beier, M., Hoheisel, J. D., Production by quantitative photolithographic synthesis of individually quality checked DNA microarrays, Vol. 28, No. 4, p. 1-6, 2000). With such methods it is only sensible to produce DNA polymer probes up to a length of 25 bases. On such an array, if the rate actually is 95%, there are still only approx. 27% full-length products. With a coupling rate of 90% per addition step, we get a value of just 7%.

To date, only the synthesis of polymer probes before arrangement on the array using a suitable purification step followed by application on the reaction support can provide almost 100% full-length probes. However, this procedure has other disadvantages, mainly logistical costs and the run-up to production including the investment in the polymer probes of a particular selected design.

Against this background, in one embodiment with asymmetric probes the objective is to avoid the aforementioned disadvantages that can arise from a population of molecules of various lengths on the individual positions of a microarray, without having to accept the disadvantages of “off-chip” synthesis.

This embodiment of the invention with asymmetric probes thus describes a method for improving the application of in-situ synthesis techniques in the production of polymer probe arrays for the method according to the invention, in which the contribution that full-length products from the synthesis process make to the analysis result is increased. This is achieved with an asymmetric configuration of the polymer probes. Especially in the last synthesis steps, modified building blocks are used, which differ in certain thermodynamic properties, e.g. binding stability, from the building blocks used previously. Alternatively or additionally, the same effect can be achieved through suitable modification of the distal end of the polymer probes, e.g. with a hybridization intensifier. Such a molecule is for example a so-called “Minor Groove Binder” (Epoch Biosciences 2000 Annual Report, pages 4-5), which markedly increases the stability of binding to the last 4-5 bases of the polymer probe. Examples of these “minor groove binders” are some natural antibiotics with a configuration that permits folding in the minor groove of a DNA helix. This replaces the purification of the polymer probes prior to application in a polymer probe array, which is lacking in in-situ syntheses. In this way the qualitative disadvantage of in-situ synthesis techniques is remedied partially or completely.

With the method described as an embodiment of the invention, the usability of polymer probe arrays synthesized in situ is improved with respect to the quality and informativeness of the analysis.

Especially for application in analyses and processes that must operate with very accurate analysis results or very precise differentiation of very similar test material, this provides further improvement of the method.

Methods and molecules for the synthesis of polymer probes with modified thermodynamic properties using modified nucleotide building blocks are known from U.S. Pat. No. 6,156,501 A. Furthermore, modifications to the finished polymer probe, which alter the binding properties of the polymer probes, e.g. intercalation of “minor groove binders” (MGB), are known from the literature. Modified synthetic building blocks are for example ribonucleoside analogs, such as LNAs (locked nucleic acids), modified purine or pyrimidine bases, such as superstabilizing adenosine analogs (e.g. 2,4-diaminoadenosines), pyrazolopyrimidines (e.g. PPG) and phosphate backbone analogs, e.g. methyl phosphonates, phosphorothionates, phosphoroamidates etc.

Other duplex stabilizers that can be used are building blocks that can lead to formation of a triple helix by a third nucleic acid or peptide strand, and stabilizing molecules, for example intercalators, which insert between the base stacking of a DNA double strand.

Another aspect of the invention is combination of the asymmetric probe design with in-situ purification methods, in which the termination products of probe synthesis are removed in situ. Post-synthetic array optimization is made possible in this embodiment by the modified building blocks at the end of the polymer probes extended right up to the end. For the most part, shorter probes do not have these modified building blocks and can be removed by suitable methods, e.g. chemical and/or enzymatic digestion.

One object of the invention is therefore a method of production of a support for the determination of nucleic acid analytes by hybridization, comprising the steps: (a) provision of a supporting material and (b) stepwise construction of an array of several different receptors selected from nucleic acids and nucleic acid analogs on the support by spatially specific and/or time-specific immobilization of receptor building blocks at respective predetermined positions on the or in the supporting material, wherein several different sets of synthetic building blocks are used for the synthesis of the receptors, in order to obtain receptors that are asymmetric, i.e. consist of several different types of receptor building blocks.

The different sets of building blocks are selected in such a way that the individual building blocks are equal with respect to the specificity for complementary nucleic acid building blocks from the analyte, but have different affinity for complementary nucleic acid building blocks from the analyte, so that preference for full-length products of a polymer probe array synthesized in situ is achieved by a special distribution of different types of building blocks along the polymer probes during the synthesis.

Preference for full-length products of a polymer probe array synthesized in situ is preferably achieved by a special distribution of different types of building blocks along the polymer probes during the synthesis. For this, for the method according to the invention, sets of synthetic building blocks are used that display the same behavior with respect to certain parameters, but differ from one another in certain, e.g. thermodynamic, properties. The distribution of the building blocks along the growing polymer during in-situ synthesis is selected in such a way that the full-length products with number of building blocks n or at least the synthesis products from the last addition steps of polymer extension contain modified building blocks. The number of building blocks n can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70.

In a preferred embodiment, at least for the last step or the last steps, e.g. the last two, three or four steps, during construction of the receptors a set of synthetic building blocks is used that has a higher affinity for complementary nucleic acid building blocks from the analyte than those used previously.

Furthermore, in one embodiment it is possible for the set of synthetic building blocks used for the last step or steps in the construction of the receptors additionally to have greater resistance to degradation reagents, e.g. enzymes, such as nucleases and/or chemical reagents, such as acids or bases, in comparison with the set of synthetic building blocks used for the first steps of the construction of the receptors. In this case, for example after completion of receptor synthesis, a special degradation step can be carried out, by which the proportion of non-full-length products is reduced relative to the proportion of full-length products. The insertion of “degradation-resistant” building blocks and a subsequent degradation step can moreover also take place once or several times during earlier steps of receptor synthesis.

An alternative or supplementary procedure envisages the production of different hybridization affinities for individual sets of building blocks by using modifications of the receptors, e.g. by means of hybridization intensifiers, by which their properties are altered in the desired manner in favor of the full-length products. The incorporation of hybridization intensifiers is spatially specific, i.e. an increased hybridization affinity for complementary nucleotide building blocks from the analyte is provided for a predetermined number (i.e. a set) of individual building blocks from the receptor. Preferably the hybridization intensifier is attached at the distal end of the receptor, for example with the last 3-5 bases of the receptor being modified with respect to hybridization affinity.

In a preferred embodiment of the method according to the invention, a nucleic acid array, selected from DNA or RNA arrays, in particular a DNA array, is constructed, wherein a first set of synthetic building blocks, consisting of unmodified DNA or RNA synthetic building blocks, which are advantageously in the form of suitable derivatives with phosphoroamidites, H-phosphonates etc., is used. A set of synthetic building blocks selected from N3′-P5′-phosphoroamidate (NP) building blocks, locked nucleic acid (LNA) building blocks, morpholinophosphorodiamidate (MF) building blocks, 2′-O-methoxyethyl (MOE) building blocks, 2′-fluoro-arabino-nucleic acid (FANA) building blocks, phosphorothioate (PS) building blocks, 2′-O-methyl (OMe) building blocks or peptide nucleic acid (PNA) building blocks is then used as the second set for the last step or steps of receptor construction. However, the method according to the invention is of course also suitable for the construction of modified nucleic acid arrays, using a first modified set of building blocks as the first set of building blocks and a second modified set of building blocks as the second set, wherein the two sets of building blocks, as previously described, differ with respect to affinity for complementary nucleic acid building blocks of the analyte and optionally additionally with respect to resistance to degradation reagents.

This variant of the method according to the invention avoids the purification problems for in-situ polymer probes by means of asymmetric configuration of the probes, which in the case of nucleic acids leads to an increased contribution of the full-length products to the binding energy in the double strand during subsequent application on the biochip.

This variant of the method according to the invention is suitable, along with the other uses described in this disclosure, particularly for the detection and/or isolation of nucleic acids, e.g. for carrying out de novo sequencing, resequencing and point mutation analyses, e.g. SNP analyses and the detection of new SNPs. Furthermore the method can be used for the analysis of genomes, genome variations, genome instabilities and chromosomes and for gene expression or transcriptome analysis or for the analysis of cDNA libraries. The method is also suitable for the production of substrate-bound cDNA libraries or cRNA libraries. Furthermore, arrays can be constructed for the production of synthetic nucleic acids, nucleic acid double strands and synthetic genes.

Moreover, arrays of PCR primers, probes for homogeneous assays, molecular beacons and hairpin probes can be produced.

Finally, arrays can also be made for the production, optimization or development of antisense molecules.

The method according to the invention is especially suitable for the production of supporting materials with channels, e.g. with closed channels. In one embodiment the channels are microchannels with a cross-section of e.g. 10-1000 μm. Examples of suitable supporting materials with channels are described in WO 00/13018. Preferably, a supporting material is used that is at least partially optically transparent and/or electrically conductive in the region of the positions that are to be fitted with receptors.

This variant of the method according to the invention is also especially suitable as an integrated method of synthesis and analysis, i.e. the finished support is used in situ for analyte determination and then optionally is used for further synthesis-analysis cycles, as described in WO 00/13018.

Furthermore, this variant of the method according to the invention also relates to a support for the determination of analytes that contains a large number, preferably at least 100 and especially preferably at least 500, of different immobilized receptors, wherein the receptors are in each case constructed from several different, e.g. two or even more sets of synthetic building blocks, and wherein the individual synthetic building blocks are the same with respect to the specificity for complementary nucleic acid building blocks from the analyte, but have different affinity for complementary nucleic acid building blocks from the analyte.

Furthermore, this variant of the method according to the invention relates to a reagent kit, comprising a supporting material and at least two different sets of building blocks for the synthesis of receptors on the support. Furthermore, the reagent kit can also contain reaction fluids.

This variant of the method according to the invention also relates to a device for integrated synthesis and analyte determination on a support, comprising a programable light source matrix, optionally a detector matrix, a support preferably arranged between the light source and detector matrix when using a detector matrix and means for feeding fluids into the support and for withdrawing fluids from the support and optionally reservoirs for synthesis reagents and samples. The programable light-source or exposure matrix can be a reflection matrix, a light valve matrix, e.g. an LCD matrix, or a self-emitting exposure matrix. These light matrices are disclosed e.g. in WO 00/13018. The detector matrix, e.g. an electronic CCD matrix, can be integrated in the supporting material as an option.

The construction of the receptors on the support can comprise fluid-chemical synthesis steps, photochemical synthesis steps, electrochemical synthesis steps or combinations of two or more of these steps. An example of the electrochemical synthesis of receptors on a support is described in DE 101 20 663.1. An example of a hybrid method, comprising a combination of fluid-chemical steps and photochemical steps, is described in DE 101 22 357.9.

The invention will be explained further with the following example.

A DNA microarray is synthesized to a length of the DNA probes of 25 building blocks. For the last building block, instead of a natural nucleotide, an analog with suitable properties is condensed on the probe. This can be an LNA (locked nucleic acid) building block, for which it is known that on the one hand it can be produced for all four bases of DNA (and therefore a set of suitable building blocks is provided), and on the other hand for all four bases, with significantly higher melting point, it hybridizes to its complementary target molecule. The discrimination between hybridization or binding to the full-length product with length of 25 building blocks in comparison with the termination products with 24 or fewer nucleotides is thereby improved. This has a beneficial effect on the analysis result.

In another embodiment, DNA or other nucleic acid polymer probes are used, which are all or partially capable of forming desirable three-dimensional structures. These three-dimensional structures can be hairpin structures or other structures known by a person skilled in the art. In this embodiment the invention comprises arrays of nucleic acids immobilized on a support, which are at least partially in the form of secondary structures, such as hairpin structures. Furthermore, methods of production of said arrays and uses thereof are claimed.

A binding event between immobilized receptor and analyte is usually detected by detection of a labeling group that is bound to the analyte. A support and a method of analyte determination, which permit integrated synthesis of receptors and analysis, are for example described in WO 00/13018. In order to use receptor arrays, e.g. DNA chips, for tackling complex biological problems (gene expression studies, target validation, sequencing by hybridization, resequencing), it is of fundamental importance that execution of the hybridization between receptor and target can be as error-free as possible. The detection system must therefore be able to differentiate between a so-called “full match”, i.e. when probe and target are completely complementary, and a “mismatch”, when one or more defective base pairings are present. Naturally it is especially difficult to differentiate between a “single mismatch”, when only 1 base pairing is defective, and a “full match”. Moreover, for thermodynamic reasons, terminal base mispairings in particular can only be detected inadequately or with difficulty. Conversely, mispairings in the middle of a sequence are easier to detect, for the same reasons.

On known DNA chips, nucleic acid receptors are in single-stranded form as far as possible. During selection of the sequences for the receptors attention is therefore directed at avoiding possible formation of secondary structures. Base mispairings are now detected by applying, on the DNA chip, not only the actual sequence requiring elucidation, but also as comparison the corresponding sequence with a mispairing in the middle of the succession of bases as negative control. Whether it is a “full match” or “mismatch” can be detected from the signal intensities, different in each case, which are produced by hybridization of the sample (target) to the probe or its negative-control sequence (mismatch sequence). However, as it is once again not always possible to make an unambiguous decision, for detecting a particular DNA sequence within the target it is necessary to use not just a single sequence, but several sequences (e.g. 20 sequences per gene) (R. Lipschutz et al., Nature Genetics, 1999, 21, 20 ff.), which are produced in each case as fragments of the sample to be detected, with the associated control sequences in each case (mismatch sequences). Accordingly, detection of a single sample sequence requires not only a single nucleic acid sequence on the DNA chip, but it usually also requires 20 sequences plus the respective 20 negative control sequences (mismatch sequences). This results in a considerable extra expense in the production of DNA chips and lowers their information density significantly.

At present there are still no possible means of detecting terminal base mispairings on DNA chips.

A problem facing this embodiment of the invention is therefore to provide a system that makes it possible to detect base mispairings very accurately. Furthermore, the system according to the invention should recognize not only base mispairings in the middle of a sequence, but also at the end (terminally) on an array in highly parallel conditions.

This problem is solved by providing receptor arrays that contain nucleic acid receptors at least partially in the form of hairpins.

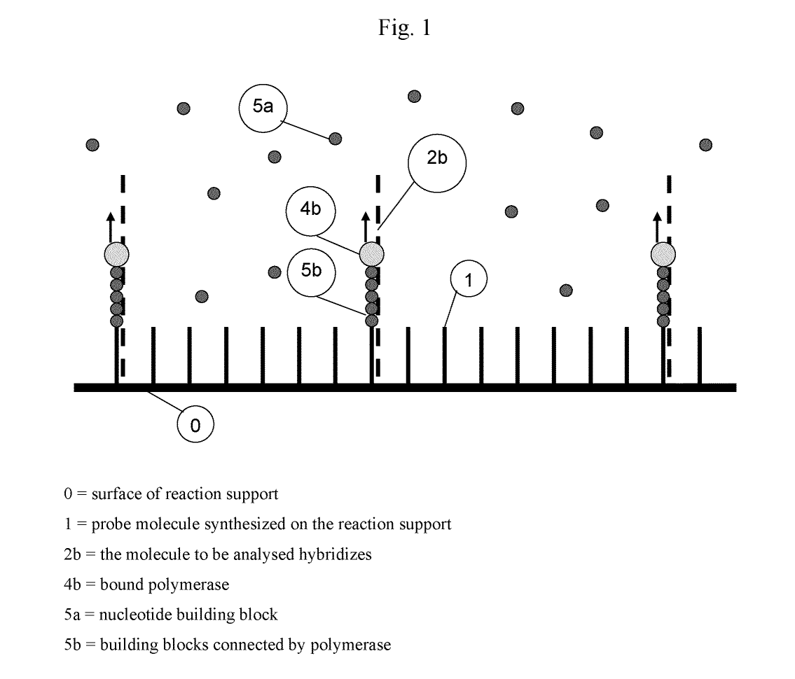

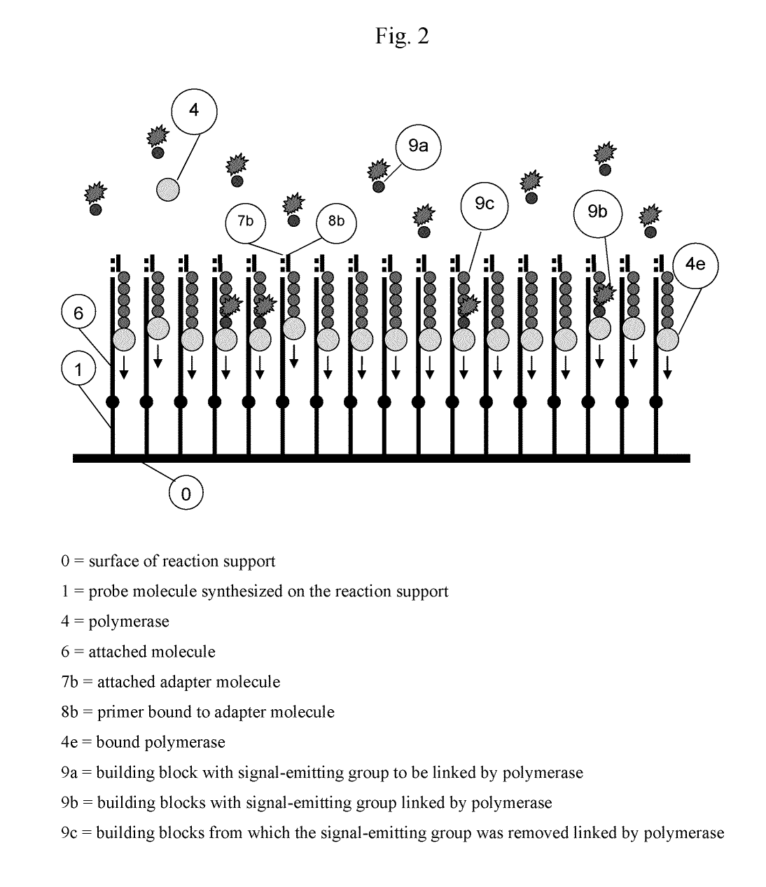

Hairpins are a special form of secondary structures in nucleic acids, which are composed of two complementary sequence segments in the so-called stem and another sequence segment in the so-called loop (FIG. 1a). There is equilibrium between the closed form and the open form (FIG. 1b). Hairpin structures have already been used in solution for marker-free detection of hybridization events (Tyagi et al. Nature Biotechnology 1995, 14, 303-308). These hairpin structures (FIG. 2 type A) are characterized in that the recognition sequence is located in the loop of the hairpin (Marras et al. Genetic Analysis; Biomolecular Engineering, 1999, 14, 151-156). In a special embodiment, in the closed state a quencher molecule and a fluorophor molecule are in close proximity, so that no fluorescence is emitted. If a hybridization event now occurs with the recognition sequence located in the loop, the hairpin opens, so that fluorophor and quencher are spatially separated from one another. Consequently a fluorescence signal can be observed. In addition to known dyes, poly-deoxyguanosine sequences can also function as quencher (M. Sauer, BioTec, 2000, 1, 30 ff.). This has the advantage that the hairpin structure only has to be labeled with one fluorophor (M. Sauer et al., Anal. Chem. 1999, 71 (14), 2850 ff.), and incorporation of a quencher molecule is unnecessary.

Studies of the behavior of hairpin structures on a solid phase—i.e. arrays with hairpin structures—are certainly known (U.S. Pat. No. 5,770,772), but they only utilize the presence of the double-stranded structure of a hairpin as a recognition site e.g. for proteins. Detection of analyte binding through opening of the hairpin structure has not been disclosed. In particular, no hairpin structures are known that utilize the sequence information in the stem of the hairpin as the recognition sequence for hybridization. Moreover, to date, no hairpin structures are known whose binding to the solid phase does not take place via a terminal end.

One object of the invention, in this embodiment, is a method for the determination of analytes, comprising the steps:

a) provision of a support with several predetermined regions, on which in each case different receptors, selected from nucleic acids and nucleic acid analogs, are immobilized, wherein in one or more of the predetermined regions, the receptors, in the absence of an analyte that can bind specifically to them, are at least partially in the form of a secondary structure. Partially relates in this case to each individual receptor, in that the presence of the respective analyte that can bind specifically to it causes a change or removal of the secondary structure in the receptor;

b) contacting the support with a sample that contains analytes, and

c) determining the analytes from their binding to the receptors immobilized on the support, wherein the binding of an analyte to a receptor that can bind specifically to it comprises the detection of the opening of the secondary structure that is present in the absence of the analyte.

Another object of the invention is a device for the determination of analytes, comprising

a) a light source matrix,

b) a support with several predetermined positions, on which receptors that are different in each case are immobilized on the support,

c) means for supplying fluids to the support and for withdrawing fluids from the support and

d) a detection matrix comprising several detectors, which are assigned to the predetermined positions on the support.

The hairpin structures according to the invention can be used, surprisingly, for very precise discrimination of base mismatches on a solid phase, in particular on an array. The hairpin structures according to the invention can be produced in situ on the solid phase, but also, if prepared previously, can be immobilized thereon, in highly parallel conditions.

The receptors are selected from nucleic acid biopolymers, e.g. nucleic acids such as DNA and RNA or nucleic acid analogs such as peptide nucleic acids (PNA) and locked nucleic acids (LNA) and combinations thereof. Especially preferably, nucleic acids are determined as analytes, wherein the binding of the analytes comprises a hybridization. However, the method also makes possible the detection of other receptor-analyte interactions, e.g. the detection of nucleic acid-protein interactions.

This variant of the method according to the invention preferably comprises a parallel determination of several analytes, i.e. a support is prepared that contains several different receptors, which in each case can react with different analytes in a single sample. The number of different receptors on one support is preferably at least 50, more preferably at least 100, still more preferably at least 200, still more preferably at least 500, still more preferably at least 1000, still more preferably at least 5000, still more preferably at least 10 000, still more preferably at least 50 000. Preferably at least 50, preferably at least 100 and especially preferably at least 200 analytes are determined in parallel.

The receptors can be immobilized on the support by covalent bonding, noncovalent self-assembly, charge interaction or combinations thereof. Covalent bonding preferably comprises the provision of a support surface with a chemically reactive group, to which the initial building blocks for receptor synthesis can be bound, preferably via a spacer or linker. Noncovalent self-assembly can take place for example on a precious metal surface, e.g. a gold surface, by means of thiol groups, preferably via a spacer or linker.

The present invention is preferably characterized in that the detection system for analyte determination combines a light source matrix, a microfluidic support and a detection matrix in an at least partially integrated structure. This detection system can be used for integrated synthesis and analysis, in particular for the construction of complex supports, e.g. biochips, and for the analysis of complex samples, e.g. for genome, gene expression or proteome analysis.

In an especially preferred embodiment the receptors are synthesized in situ on the support, for example by directing fluid with receptor-synthesis building blocks over the support, immobilizing the building blocks on respective predetermined regions on the support spatially and/or time-specifically, and repeating these steps until the desired receptors have been synthesized on the respective predetermined regions on the support. This receptor synthesis preferably comprises at least one fluid-chemical step, a photochemical step, an electrochemical step or a combination of said steps and online process monitoring, for example using the detection matrix.

The light source matrix is preferably a programmable light source matrix, e.g. selected from a light valve matrix, a mirror array, a UV-laser array and a UV-LED (diode) array.

The support is preferably a flow cell or a microflow cell, i.e. a microfluidic support with channels, preferably with closed channels, in which the predetermined positions with the respective differently immobilized receptors are located. The channels preferably have diameters in the range from 10 to 10 000 μm, especially preferably from 50 to 250 μm and can basically be configured in any shape, e.g. with circular, oval, square or rectangular cross-section.

As already mentioned, in this embodiment of the invention the secondary structures preferably comprise a hairpin structure, which is composed of a stem and a loop. In a first embodiment of the method according to the invention, the sequence of the receptor that is able to bind to the analyte can be located in the region of the loop of a hairpin. Binding of the loop to the receptor causes the hairpin structure to open. This opening of the hairpin can in its turn be detected by suitable means (e.g. see above). In an especially preferred embodiment, however, the sequence of the receptor that binds specifically to the analyte is located in the stem of the hairpin structure. Also in this embodiment, the binding of the analyte to the receptor causes a detectable opening of the hairpin structure.

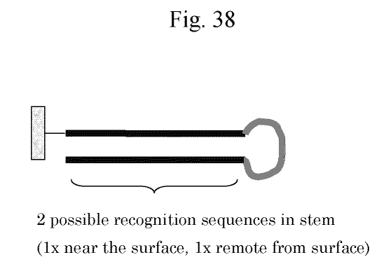

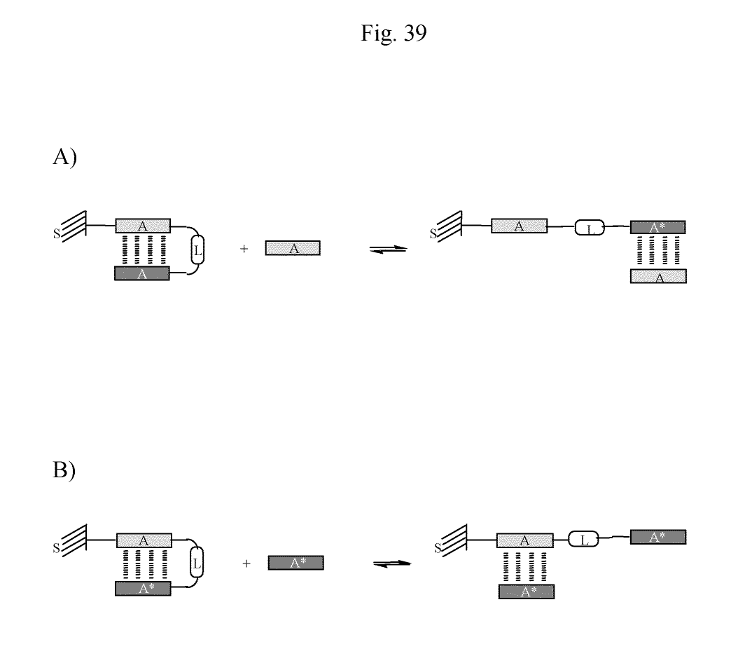

According to a preferred embodiment, the hairpin structures according to the invention with a recognition sequence in the stem (FIGS. 38, 39A and 39B) comprise complementary sequences A and A* in the stem and a linker unit L in the loop. The loop of the hairpin contains building blocks that cannot enter into any base pairings (e.g. polyethylene glycol, alkyl, polyethylene glycol phosphate or alkyl phosphate units) or building blocks that can only enter into weak base pairings (e.g. a Tn-loop with n=2-8). Both sequence segments A (FIG. 39B) or A* (FIG. 39A) in the stem can serve as recognition sequences. If a hybridization experiment is carried out, then for example a sequence A contained in the sample to be investigated competes with the reference sequence A in the stem of the hairpin for the sequence A* (FIG. 39A). This competitive situation is utilized for increasing the specificity of hybridization. If for example the sequence A contained in the sample is not completely complementary to A* (i.e. mispairings occur), the pairing between the two sequences A and A* in the hairpin is more stable, with the result that the hybridization equilibrium is displaced to the left side to the closed form of the hairpin (FIG. 39A). Thus, if a labeled sample A is used for the hybridization, this means that no signal or only a small signal can be detected, as the equilibrium is on the side of the closed hairpin. Signals can only be detected when the hairpin structure is in the open state, i.e. stable pairing is possible between A in the sample to be investigated and A* in the hairpin, and the equilibrium is on the right, i.e. with an open hairpin.

Therefore apart from the usual variables of the conditions of stringency, e.g. salt concentration, temperature, concentration of probe and target, another variable is introduced, which can have an influence on the stringency of a hybridization experiment. Furthermore, the hybridization equilibrium (and therefore the stringency) of the reference probe or of the recognition sequence, among others, can be varied so that these sequences contain building blocks of nucleic acid analogs, which are characterized in that they bind more strongly to DNA, than DNA to DNA. For this, consideration can be given to, among others, PNA or LNA building blocks or other building blocks with the described characteristics known by a person skilled in the art.

With the procedure described, this means that in contrast to the usual procedure (use of 1 perfect-match probe+1 single-base-mismatch probe) for discrimination between perfect-match and single-base-mismatch, only a single probe needs to be used, and therefore fewer positions are required on the array, or more information can be acquired with a given quantity of positions. Moreover, this also means that terminal mismatches can be queried, because owing to the presence of the reference sequence in the same molecule, higher conditions of stringency can be established, than when 2 separate probes are used for the discrimination of perfect matches and single-base mismatches.

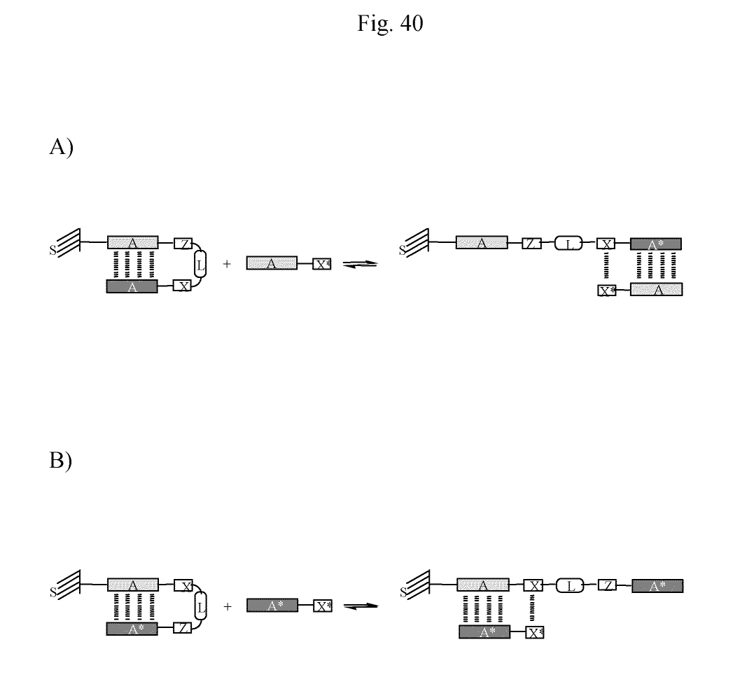

In another preferred embodiment the hairpin structures comprise two complementary sequences (A, A*) and two noncomplementary units (Z, X) in the stem and a linker unit (L) in the loop (FIG. 40). Both the sequence A-Z near the solid phase (FIG. 40A) and the sequence A*-Z remote from the solid phase (FIG. 40B) can serve as recognition sequences. What is of decisive importance is the fact that X and Z do not pair with each other. For this, it is proposed according to the invention that X represents one or more nucleic acid building blocks capable of pairing, and Z represents one or more building blocks not capable of pairing. Z can for example be an “abasic site” (DNA or RNA building block without heterobase) or a building block known by a person skilled in the art, which does not enter into base pairing, but does not disturb the DNA structure. L is to be understood as a linker that preferably consists of nucleic acid building blocks not capable of pairing, e.g. polyethylene glycol phosphate units (R. Micura, Angew. Chemie, 2000, 39(5), 922 ff.) or building blocks that can only engage in weak base pairings (e.g. a Tn loop with n=2-8). As a result, terminal mismatches in particular are more easily detected. This is because in this embodiment (FIG. 40A) further bases are available for pairing for the target A-X*, but not for the reference A-Z. As a result, the equilibrium between closed form of the hairpin (left) is displaced advantageously to the right side (open hairpin), if additional base pairing of the target A-X* with the sequence region X in the hairpin can take place. If mispairings occur between target A-X* and the sequence region X in the hairpin, this is not so, and the equilibrium is displaced unintensified to the right (open) side.

Owing to the presence of additional segments X, capable of pairing, in the hairpin structure of the receptor, which are complementary to the analyte to be detected, the stringency of the hybridization experiment can therefore be further increased (FIGS. 40A and 40B). Once again, nucleic acid analogs, as previously described, which bind more strongly to DNA, than DNA to DNA, can be used.

In a further embodiment, Z can also be a mixture of the 4 bases adenosine, guanosine, cytidine and thymidine or uracil.

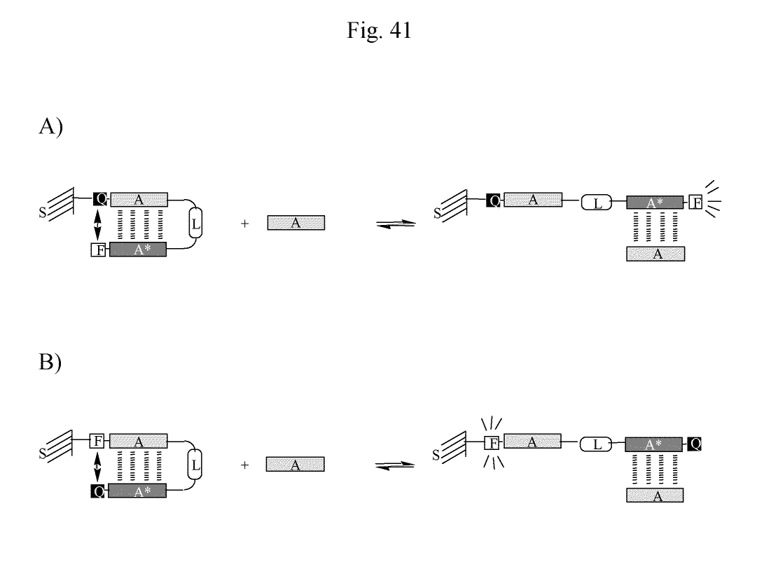

In yet another embodiment, the hairpin structure contains a labeling group that is at least partially quenched in the closed state, e.g. a fluorophor. When the hairpin structure opens, the signal originating from the labeling group increases and this increase in signal is detected. Thus, a hairpin structure according to the invention (FIG. 41) contains for example a quencher (Q) and a fluorophor (F), which are located at opposite ends of the nucleic acid sequence of the hairpin. According to the invention, the fluorescence in the closed hairpin is quenched by the close proximity of Q and F. In the open state, fluorescence is detectable. Combinations of molecules Q and F are sufficiently well known by a person skilled in the art.

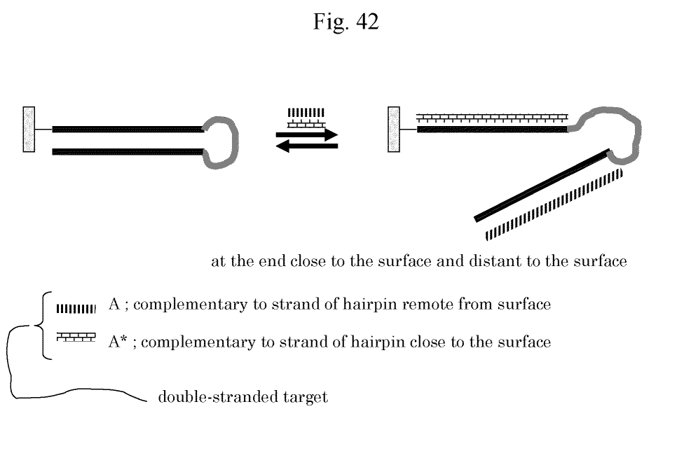

In yet another embodiment, hybridization can also take place with double-stranded targets (FIG. 42). If both strands are labeled, this can intensify the luminous intensity detectable for one position.

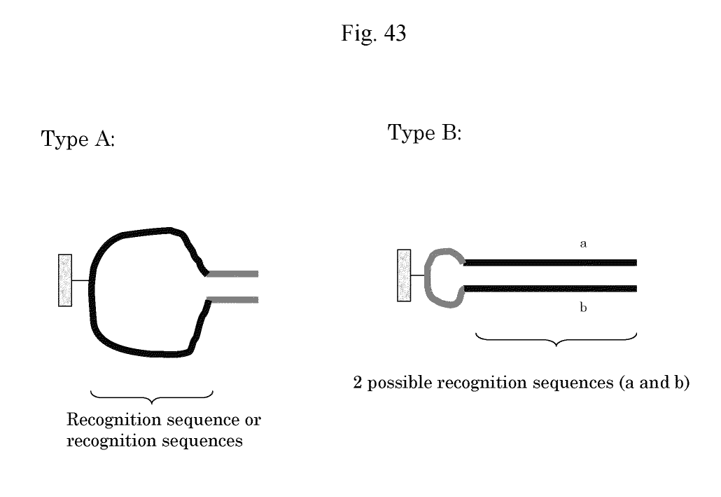

In another embodiment, the attachment of the hairpin structures to the support, both of type A (recognition sequence in the loop) and of type B (recognition sequence in the stem), can take place not only terminally, but also internally (FIG. 43). Hairpin structures of type A bound internally to the support are also disclosed hereby. Combinations of terminally and internally immobilized receptors on a support are also possible.

An improvement relative to the prior art is achieved in one embodiment especially in that the hairpin structure according to the invention has the recognition sequence in the stem. The complementary strand to the recognition sequence is used as reference sequence for mismatch discrimination. In a hybridization experiment, a target sequence present in the sample solution competes with the reference sequence (A*) for the probe sequence (A). If desired, the stringency can be further increased by inserting special nucleic acid building blocks (PNA, LNA) in the reference strand. That is, only one hybridization will take place—i.e. the hairpin will change to the open form—if the target sequence present in the sample solution is exactly complementary to the probe sequence. If this is not so, the reference sequence integrated in the hairpin ensures that the hairpin does not change to the open form, and therefore hybridization to a target sequence cannot take place.

Moreover, the hairpin structure according to the invention permits the discrimination of terminal base mismatches. These become possible through the hybridization of the target sequence to position X. Base pairings complementary to the terminal position X determine whether the hairpin changes more intensively to the open form and as a result a hybridization event can be detected.

As a result, in contrast to conventional DNA arrays for deciding whether it is a “full match” or “mismatch”, far fewer sequences have to be applied. The result is that a larger quantity of different target sequences can be processed with the maximum density of positions of an array defined a priori. This increases the information density of the array significantly.

By incorporating fluorescence (F) and/or quencher (Q) building blocks in the hairpin structure according to the invention, moreover, a marker-free detection of DNA arrays can be achieved (FIG. 41).

In another embodiment of the invention using the probes described above with secondary structures and hairpins, an integrated system for the determination of analytes is prepared, which permits highly parallel in-situ production of complex populations of hairpin receptors immobilized in microstructures for the detection of analytes.

This employs, advantageously, a device comprising:

a) a light source matrix,

b) a microfluidic support with several predetermined positions, on each of which different receptors, selected from nucleic acids and nucleic acid analogs, are immobilized on the support, wherein in one or more of the predetermined regions the receptors, in the absence of an analyte that can bind specifically to them, are at least partially in the form of a secondary structure,

c) means for supplying fluids to the support and for withdrawing fluids from the support and

d) a detection matrix, comprising several detectors, which are assigned to the predetermined regions on the support.

In the device according to the invention, preferably any two or any three or all four of the components a), b), c) and d) are present in integrated form. Especially preferably, the support is arranged between light source matrix and detection matrix. The detectors of the detection matrix are preferably selected from photodetectors and/or electronic detectors, e.g. electrodes.

The device according to the invention can be used for the controlled in-situ synthesis of nucleic acids, e.g. DNA/RNA oligomers, wherein photochemical, fluid-chemical, and/or electronically cleavable protective groups can be used as temporary protective groups. Positionally resolved and/or time-resolved receptor synthesis can take place by directed control of electrodes in the detection matrix, directed feed of fluid into defined regions or groups of regions on the support and/or directed illumination via the light source matrix.

All three-dimensional and completely or partially controlled or completely or partially uncontrolled secondary and three-dimensional polymer probe structures can be used for binding studies and the multistage molecular-biological processes according to the invention, in such a way that the binding of proteins, peptides, cells, cell fragments, organelles, saccharides, low-molecular active substances, complex molecules, nanoparticles, synthetic organisms or molecules or cells from synthetic biology can be analyzed and optionally optimized. In an embodiment derived from this, the binding of proteins from cell extracts or in-vitro production can be investigated, and their binding pattern can be investigated on a set of sequence motifs. This set of sequence motifs can consist of a set of binding sites for transcription factors. Furthermore, this set can consist of hypothetical or empirically validated binding sites. A mixture of hypothetical or empirically validated binding sites can also be provided.

In another embodiment, the three-dimensional and completely or partially controlled or completely or partially uncontrolled secondary and three-dimensional polymer probe structures are used for binding studies and the multistage molecular-biological processes according to the invention, in such a way that the binding pattern of microRNA, other not-protein-encoding RNA molecules or complexes consisting partially of RNA, partially of proteins or peptides with the double-stranded hairpin structures, other structures or double strands derived from the hairpin structures on the reaction support are investigated and optionally are detected from markers on the analytes.

In another embodiment, as part of the multistage molecular-biological process, a FRET reaction can be produced and can be used for further analysis or information gathering. The FRET effect can be brought about by a polymer probe-target interaction. In order to make local excitation of fluorescence possible, an acceptor or donor molecule is coupled to the constructed polymer probes. This can take place during synthesis (by means of the building blocks) or after synthesis, e.g. with reagents such as cisplatin (see e.g. KREATECH ULS-Cy5). The sample material carries the corresponding other marker, to make the FRET possible, e.g. the Cy5/Cy3 pair or phycoerythrin, or a quencher suitable for the donor. Energy transfer can only take place near the surface, so that self-fluorescence of the solution or unbound, free labeled sample material does not cause any interfering background fluorescence. In one embodiment, which can for example be used in the genotyping of nucleic acids, the probe with a free 3′ end can represent the fluorescence acceptor, and by means of a polymerase reaction as described above (e.g. a primer extension) or another enzyme reaction (e.g. a ligation), a suitable donor (EX), which makes the FRET possible, is attached to the bound complex or to the polymer probe itself. The “Big Dye” principle for “four-color sequencing” can be made possible in this way. A donor exciter molecule (fluorescein etc.) is excited and transfers its energy to the ddNucleotide dye molecule attached by primer extension. In another embodiment with FRET, the FRET reaction is made possible by inserting acceptor and donor molecule pairs in the sample material after or attachment to the polymer probe, e.g. during or by means of a primer extension reaction. This leads in this variant to high marker density, so that many FRET transfers can proceed. A particular advantage is offered by an embodiment with a combination of FRET and CCD detection, as this permits direct detection of the course of the reaction.

In another embodiment of the invention polymer probes are immobilized in or on a reaction support. This can take place covalently or noncovalently. These polymer probes are constructed from nucleic acids or analogs thereof. On these polymer probes, the sample to be investigated, which consists of at least 2 nucleic acid sequences, is immobilized by hybridization. In the controlled loading of individual reaction fields with various particular polymer probes according to the invention, the specificity of the subsequent attachment of nucleic acids from the sample may be influenced by the sequence. Thus, a reaction support can for example address the 3′ or 5′ sequence motifs of all exons of a gene family or a whole genome. In the next step, one more amplification can take place on the reaction support. In the next step the sequence of building blocks along the bound nucleic acids from the sample is determined by an enzymatic reaction. This can take place directly on the nucleic acid strands from the sample or via their amplificates or via the complementary sequence after a primer extension on the polymer probes. Methods for determining the building blocks along the nucleic acid strands are known by a person skilled in the art, by the term “sequencing by synthesis”, among others. For this, polymerases, kinases and ligases, among others, are used as enzymes. Technical implementations have been presented by the companies Agencourt, 454 and Solexa. These methods all have in common an end point of the decoding reaction, in which the reaction no longer allows sufficiently precise discrimination of correct from other signals and therefore no longer allows unambiguous classification, e.g. caused by nonspecific side reactions. This completes a first cycle in the decoding process.

Later in the process, possibly after a calibration, purification or initialization step, a mixture of at least 2 short nucleic acid strands is added. These short nucleic acid strands have a function similar to a primer molecule in the PCR reaction. They serve for a second cycle of the various embodiments of the decoding reaction along the bound nucleic acids from the sample. The sequence of the short nucleic acid strands, which perform a function similar to a primer molecule in the PCR reaction, can already have been determined before the first cycle of the decoding reaction along the bound nucleic acids. In a preferred alternative embodiment, this sequence is matched to the results of the first cycle of the decoding reaction. In this connection, “primer walking” is known by a person skilled in the art from the conventional sequencing techniques. In the method described here, an especially preferred embodiment of the invention is highly parallel primer walking on 2 or more nucleic acids from the sample by means of 2 or more short nucleic acids with sequences that are selected on the basis of the sequence determined for the 2 or more nucleic acids from the sample. In one embodiment, it may be envisaged that the sequence determination in the second cycle at first still comprises a short sequence motif that was also already determined in the first cycle, in order to derive a quality feature from it.

In a preferred embodiment it is envisaged that the short nucleic acids are obtained from a method of parallel synthesis. These parallel synthesis techniques are known by a person skilled in the art from the area of biochips and microarrays. There are electrochemical, optically controlled and fluidically controlled methods for the production of 2 or more nucleic acid sequences on one support. What is claimed here is the use according to the invention of 2 or more nucleic acid sequences from a parallel method of production, in which the 2 or more nucleic acid sequences are either produced on a common support or are produced in a process in which at least in one step 2 or more reaction sites for the production of the 2 or more nucleic acid sequences are contacted simultaneously with a reagent. Examples of said parallel methods of production for 2 or more nucleic acid sequences for the method according to the invention are DNA microarrays, which are produced by photolithography, projector technology, LED technology, emitting semiconductor components or LCOS Projection. Other examples are methods of production that use direct photochemistry, and methods of production that use indirect photochemistry, such as light-induced bases or acids. Further examples are electrochemical processes. Reference is also made to fluidic processes, which either apply the building blocks arranged on a support (printing technology) or which apply the synthetic reagents selectively.

In a variant of “parallel primer walking”, the nucleic acids from the sample can also be immobilized directly on the solid phase, e.g. on beads or particles, on a reaction support, a microscope slide, or in a layer of gel. Next there is a first cycle of the decoding reaction, followed by the second cycle described above. Other process steps can take place in-between.

### 6.16 Several Different Probes are Synthesized Per Position of the Reaction Support

By using orthogonal chemical methods for the production of molecules in the reaction support it is possible, per addressable location for the synthesis, to synthesize more than just one sequence of a particular type of molecule. For example, it is possible to produce two mutually suitable, hybridizable molecules at one location, that bind to one another and form a hybrid. These can be, for example, a DNA double strand or double strands from nucleic acid analogs or derivatives. The synthesis of DNA oligonucleotides or derivativized DNA oligonucleotides or DNA oligonucleotide analogs with different sequences at one location is preferred. It is possible for 2, 3, 4, 5, or 6 different sequences to be synthesized per addressable location for the synthesis. The synthesized molecules can be synthesized with different total surface concentration or different individual surface concentrations. The ratio of the amounts of the different molecules is variable. For example, for the production of such locations, containing DNA oligonucleotides or derivativized DNA oligonucleotides or DNA oligonucleotide analogs with different sequences, various building blocks with mutually orthogonal chemical protective groups are applied first, as known by a person skilled in the art. The ratio of these building blocks is entirely optional and is preferably 10/1, 9/1, 8/1, 7/1, 6/1, 5/1, 4/1, 3/1, 2/1, 1/1 or vice versa, but can also be between these values (1/1-10/1). One type of the mutually orthogonally protected building blocks applied to the surface is now selectively deprotected, i.e. the protective group is split off, so that only this one type of building block is available for further reaction. On these deprotected building blocks, a sequence of DNA oligonucleotides or derivativized DNA oligonucleotides or DNA oligonucleotide analogs is now constructed by methods of nucleic acid solid-phase synthesis known by a person skilled in the art, and then protected once again. This protection is orthogonal to the deprotection conditions of the other mutually orthogonally protected building blocks previously linked to the surface of the reaction support. Now another type of these building blocks previously linked to the surface of the reaction support is deprotected, wherein other protective groups in the reaction support are preferably not also split off. Now only this one type of building block is available for further reaction. On these deprotected building blocks, a second sequence of DNA oligonucleotides or derivativized DNA oligonucleotides or DNA oligonucleotide analogs is now constructed by methods of nucleic acid solid-phase synthesis known by a person skilled in the art. This process can be repeated for each individual type of mutually orthogonally protected building blocks previously linked to the surface of the reaction support, and thus several different sequences are produced at one location.

6.17 Probes with a Labile Linker are Synthesized in the Reaction Support

In another preferred embodiment, molecules are synthesized on the surface of the reaction support, which are bound to the surface of the reaction support via a unit that is cleavable under defined conditions (labile linker, labile spacer). Using particular methods, these units can now be cleaved and the molecules synthesized on the surface of the reaction support are thus removed from the surface. Cleavage can for example be effected by changes in temperature or pH value, by irradiation with light and/or by adding chemicals, for example acids, bases, nucleophiles, electrophiles, radicals, ions or catalysts, enzymes and others. The rate of the cleavage reaction can be controlled and the complete cleavage reaction can take hours and/or days. The cleavage reaction is preferably carried out in such a way that it is carried out simultaneously with an analytical method in the reaction support. For example, an enzymatic reaction can be carried out in the reaction support, while a proportion of the molecules is slowly split off from the surface and is only then available to the enzyme as reaction partner or binding partner or as substrate. In this way the supply of molecules can be controlled during the reaction. For example, it is thus possible to control the rate of the reaction or the amount of end product formed. The cleavable molecule can preferably be a DNA oligonucleotide or derivativized DNA oligonucleotide or DNA oligonucleotide analog, which can function as primer in an enzyme reaction in the reaction support when it is split off. Especially preferably, it is also possible to use enzymes for cleavage. Several methods that use enzymes such as uracil-DNA-glycosylase (UNG, UDG) for splitting-off DNA oligonucleotides or derivativized oligonucleotides or oligonucleotide analogs, are known by a person skilled in the art.

6.18 PCR in the Reaction Support: Control of the Binding Events by Covalent Linkage of Participating Molecules with the Reaction Support

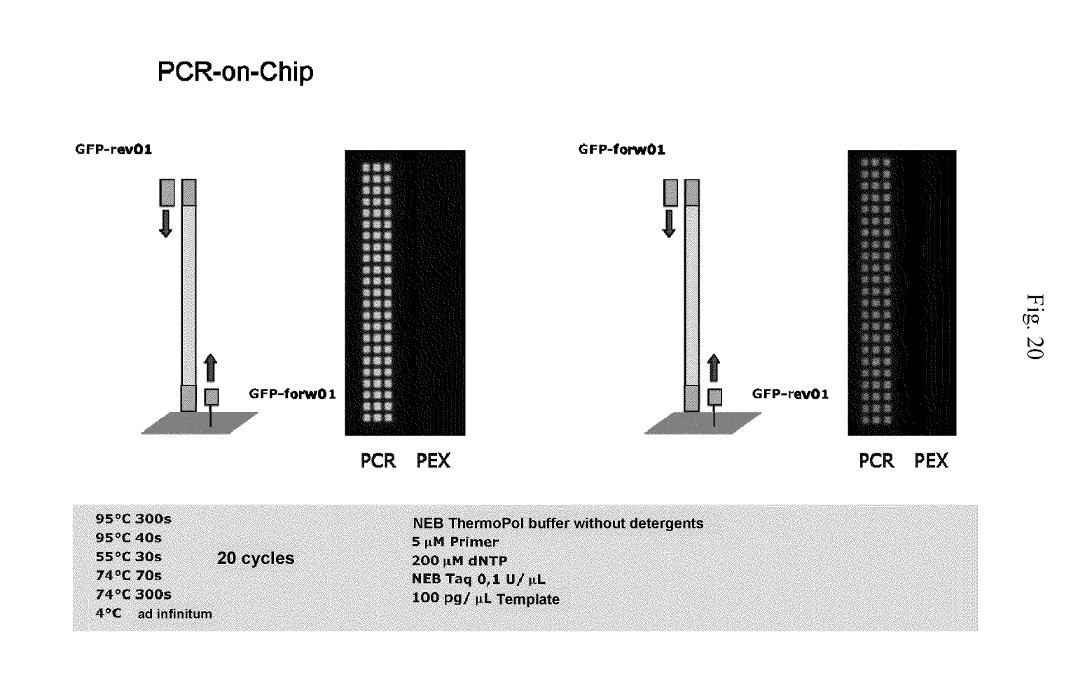

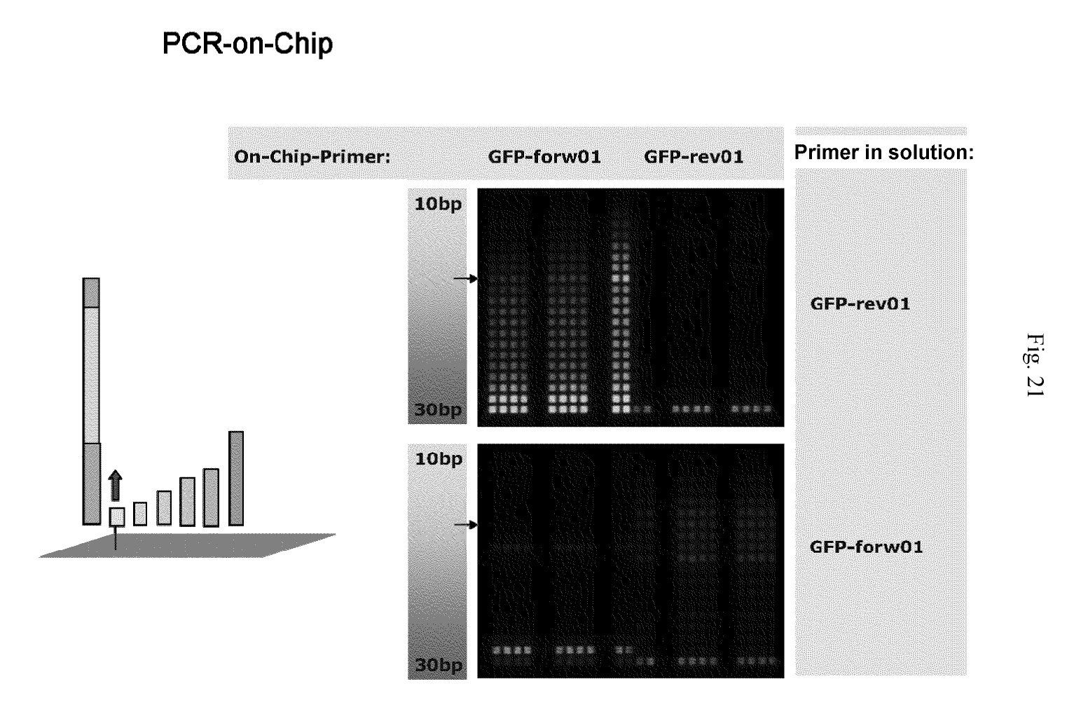

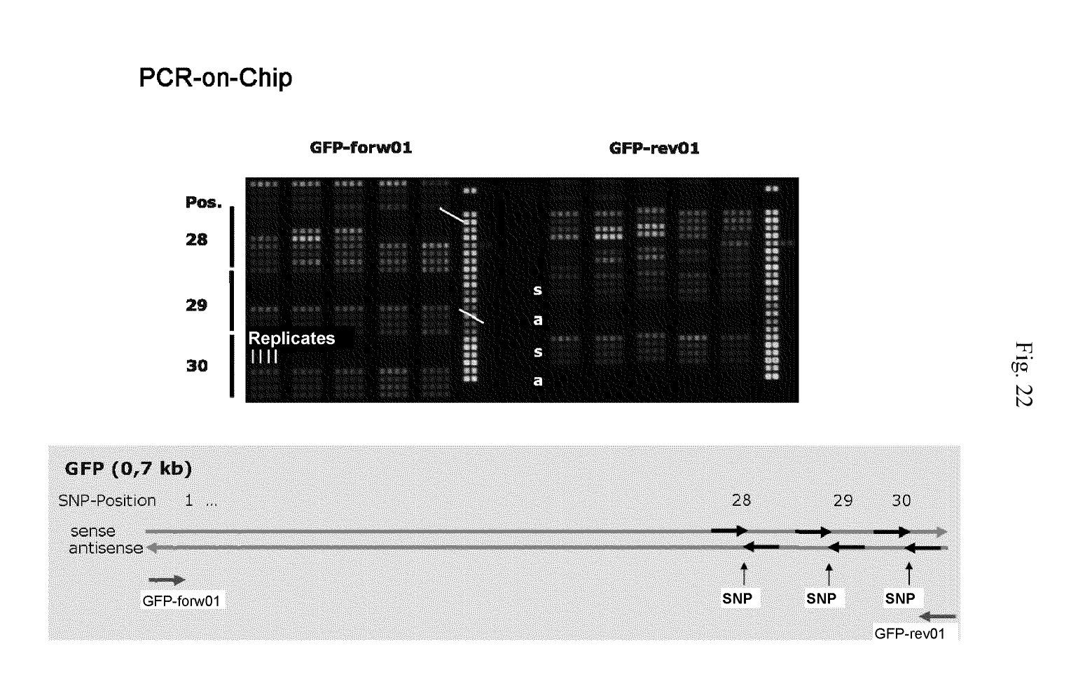

In another preferred embodiment, reactions for amplification in the reaction support, as known by a person skilled in the art, are carried out. In particular, the methods described in 6.1, 6.5, 6.6, 6.9, 6.15, 6.16, 6.18, 6.19, 6.20 and 6.21 can be used for this. A PCR is preferred, in which DNA oligonucleotides or derivatized DNA oligonucleotides or DNA oligonucleotide analogs bound to the surface of the reaction support act as primers. FIGS. 20-22 illustrate these uses and show data from successfully executed PCR reactions on the surface of the reaction support. Primers can be used which, as described in 6.16, are present at the same addressable location for the synthesis. Preferably 1, 2, 3, 4, 5, 6, 7, 8, 9 or 10 primers are used per location. Use of 2 primers is especially preferred. These primers can be linked covalently to the surface of the reaction support in such a way that they can no longer bind directly to one another. Thus, no primer-dimers known by a person skilled in the art, neither homodimers nor heterodimers, can be formed during the PCR. If longer molecules are now added to the reaction support, which are able to bind to the primers and can serve as template in the PCR, the primers are extended by a polymerase and then reach a length that permits binding of a second primer in the opposite direction. Without addition of longer molecules, this primer extension does not take place. After a second extension step, a PCR is possible in which all primers and all molecules including the longer ones added originally, functioning as template, are linked covalently to the surface of the reaction support. Therefore between different addressable locations there cannot be any cross-reactions or interactions between primers, template molecules, substrates or products of the PCR reaction. The only constituents of the reaction mixture capable of diffusion are in this case enzymes, nucleotides and other buffer constituents known by a person skilled in the art, but not oligonucleotides or other nucleic acids that are contained in the mixture. In a preferred embodiment there are two primers with different sequences at one location, which in each case bind specifically to particular sequences in complex sample mixtures. The complex sample mixtures used are preferably partially or completely purified or unpurified fragmented or unfragmented genomic DNA or partially or completely purified or unpurified fragmented or unfragmented RNA extracts from sample material. As in a PCR known by a person skilled in the art, the primers are oppositely directed and after binding on the desired sample molecule they are not more than 20000 nucleotides apart. One primer binds to the sense strand of the sample molecules known by a person skilled in the art, and the other one to the antisense strand.

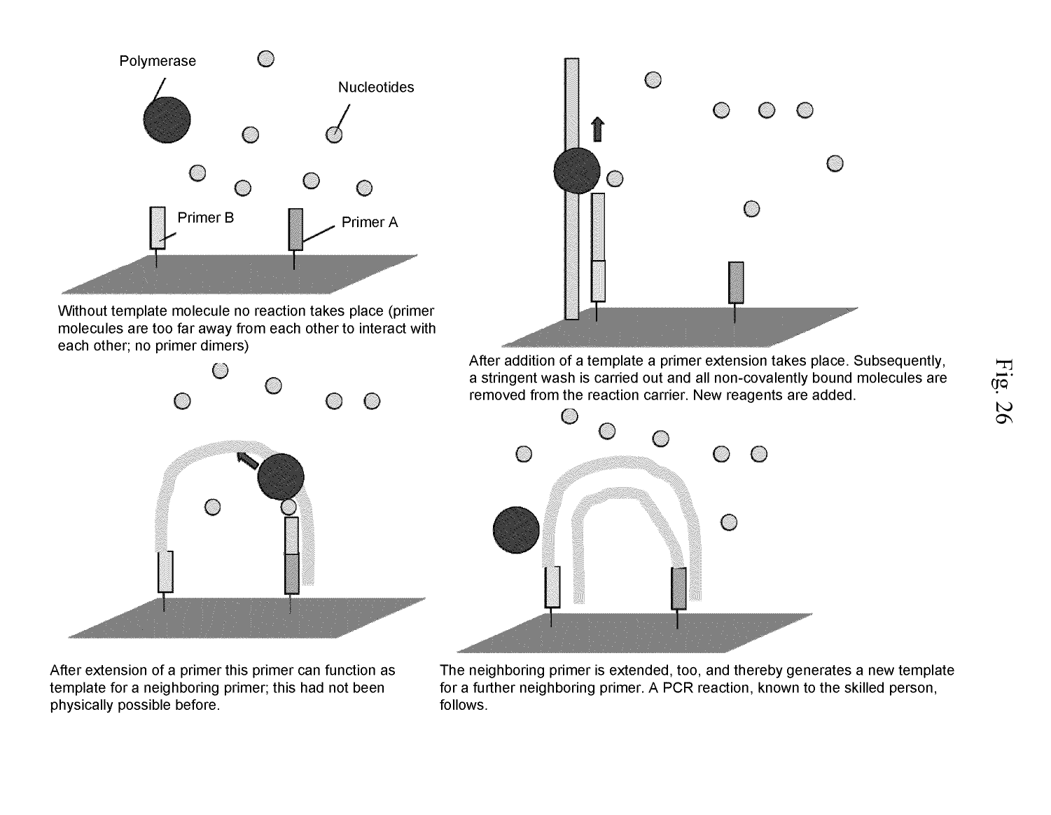

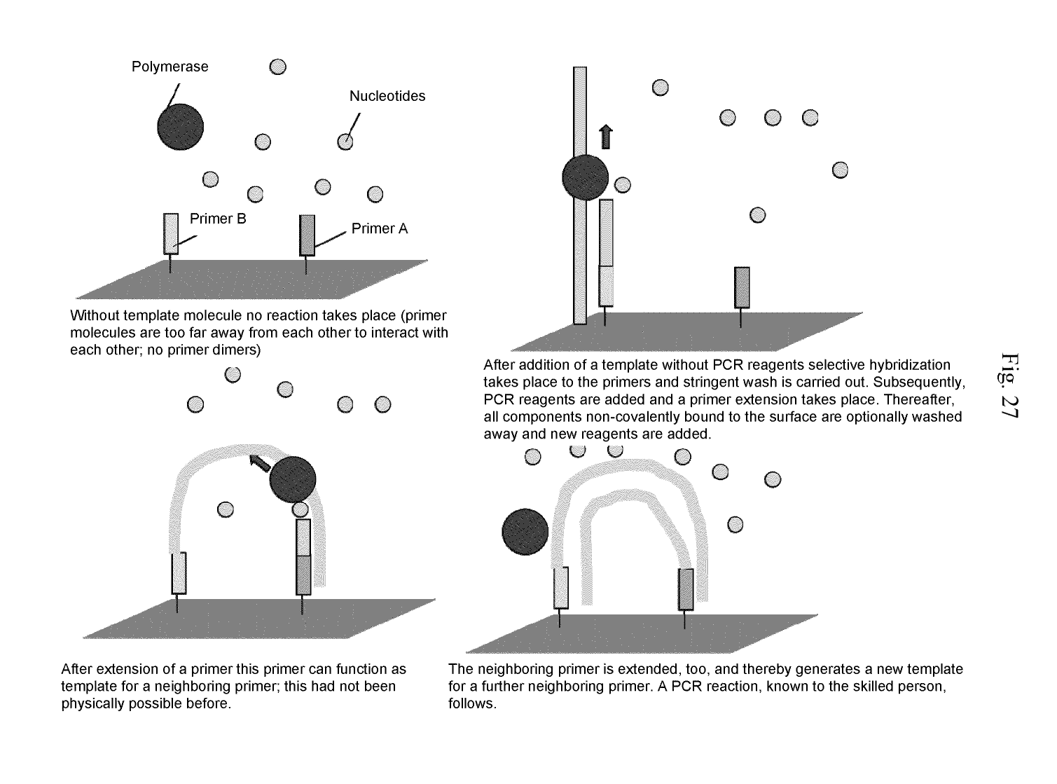

In a preferred embodiment, as a result of combining hybridization and washing steps, the desired sample molecules bind to the primer and are retained in the reaction support, whereas those not wanted are removed by washing, so that the complexity of the sample mixture can be reduced by this process. Then a so-called primer extension can be carried out by means of the primers bound to the desired sample molecules. During this, the primers are extended until they can serve as template for further, oppositely directed primers. Washing is now performed under stringent conditions in such a way that all constituents of the mixture not bound covalently to the surface of the reaction support are removed from the reaction support. Next, constituents that are necessary for a PCR and are known by a person skilled in the art are introduced into the reaction support and a PCR is carried out. FIGS. 26 and 27 illustrate this embodiment. Overall, this strategy represents a considerable improvement over the prior art, because undesirable interactions that are as a rule unavoidable, such as occur in multiplex PCRs known by a person skilled in the art, are suppressed. These include mis-hybridizations of primers to sample molecules (i.e. to undesirable sites) and of primers to one another (primer-dimers). Without requiring a prior bioinformatic calculation of primers (e.g. for the exclusion of primer-dimers), in such systems primer-dimers can in fact no longer occur, because owing to their covalent bonding to the surface, the primers can no longer bind to one another. During the PCR, all primers, template molecules and products formed by the PCR, that can hybridize to one another, to primers or template molecules, are bound covalently to the surface and are thus isolated from one another. Thus, at the individual locations on the surface of the reaction support, there is formation of simple systems of few primers and molecules bound covalently to the surface of the reaction support, which arise through the extension of these primers and can serve as template for further primers at the location. It is thus possible to carry out many thousands or hundreds of thousands of individual PCRs, isolated from one another, in the reaction support, without it being possible for undesirable cross-reactions to occur between the individual PCRs. The only soluble, freely diffusing components present in the reaction support are the constituents of a PCR reaction known by a person skilled in the art, such as buffer constituents, enzymes, building blocks such as triphosphates etc., but not specific nucleic acids or derivatives.

### 6.19 Quantitative (Allele-Specific) Real-Time PCR

In another preferred embodiment, PCR reactions are carried out in the reaction support with the participation of molecules synthesized on the surface of the reaction support, functioning as primers. The progress of the reaction, i.e. the amount of nucleic acid synthesized during the PCR reaction, is observed by particular methods during the reaction. For example, methods that are known by a person skilled in the art are used for this, such as the use of molecular beacons, scorpion primers, intercalators, minor groove binders, sunrise primers and the like. A signal, e.g. a fluorescence signal, is read at various points of time during the PCR reaction.

This signal can then be employed for quantification of the particular molecules contained in the sample mixture used. Alternatively the signals can be used to obtain information about the sequence of the molecules contained in the sample. For example, mutations can be clarified, such as SNPs, deletions or insertions known by a person skilled in the art. This method is used especially preferably in combination with the PCR method described in 6.18.

### 6.20 Ultra-Longmers

In another preferred embodiment, molecules synthesized on the surface of the reaction support are used as primers. After specific binding to particular template molecules, these are extended by primer extension, so that very long molecules form, bound to the surface of the reaction support, so-called ultra-longmers. Preferably a PCR can also be carried out with an antisense primer known by a person skilled in the art, so that the length of the extended primer after a PCR can be defined by the position of this antisense primer. After washing the reaction support under stringent conditions, a reaction support is then obtained which contains long, single-stranded probe molecules with known sequences and optionally defined lengths. Just one sequence is obtained at high purity per addressable location on the surface of the reaction support. The resultant probe molecules are then available for binding desired sample molecules from complex sample mixtures. The length of the probe molecules is preferably 50, 100, 200, 300, 400, 500, 600, 700, 800, 900, 1000, 1100, 1200, 1300, 1400, 1500, 2000, 3000, 4000, 5000 or 10000 nucleotides or nucleotide derivatives or nucleotide analogs. The aforementioned production of said very long probe molecules on the surface of the reaction support is especially preferred for probe molecules consisting of DNA. These are preferably used for binding sequences of genomic DNA. These synthesis steps can result in an array comprising a large number of these ultralongmers.

In a preferred embodiment these long fragments can be used e.g. for production of synthetic genes. If a copy of a sample material is made in the reaction support by the primer extension and/or PCR described, the reaction support can be copied once again by a primer extension and the resultant molecules bound noncovalently to the reaction support are removed from the reaction support. Alternatively techniques with a labile linker molecule as described in 6.17 can be used, and permit detachment of the extended primer molecules from the reaction support. These can be used for assembling longer fragments and for expressing or producing RNAs or proteins encoded in the sequence of the molecules in vitro or in vivo or, without prior further assembly, for expressing or producing RNAs or proteins encoded in the sequence of the molecules in vitro or in vivo or for storing them in the form of clones. Owing to the great length (100-10000 nucleotides) of the individual molecule copies of the sample material that were generated by primer extension and the large number of the individual sequences, sequence information in the range from 1000000 to 1000000000 nucleotides can thus be imaged on the reaction support. Said reaction supports can be used several times and represent a copy of the sample material used. This can in particular be used for preparing e.g. fingerprints on the basis of the DNA or RNA sequence information of e.g. pathogens, bioweapons or other organisms.

The long molecules covalently bound to the surface of the reaction support can, as they are a copy of the sample material, be sequenced directly in the reaction support instead of the sample material and therefore provide the same sequence information as sequencing of the sample material itself. Methods known by a person skilled in the art and the sequencing methods described in 2.3 and 6.5 can be used for this. Sequencing provides, at the same time, quality assurance of the reaction support generated by copying the sample material, applicable for further use of the reaction support.

### 6.21 Amplification of Molecules Through Interaction of Polymerases and Nucleases in the Reaction Support

In another preferred embodiment, molecules synthesized on the surface of the reaction support are used as primers. In this case it is possible to use so-called hairpin structures, which possess intramolecular hybridization regions, which are completely or partially double-stranded and have a free 3′ end that can be extended by a polymerase. Alternatively primer molecules can be hybridized to the molecules synthesized on the surface of the reaction support and can be extended by a polymerase. Prior to this, using particular methods, the primers can be linked (crosslinked) covalently to the molecules synthesized on the surface of the reaction support. Psoralen, for example, can be used here.

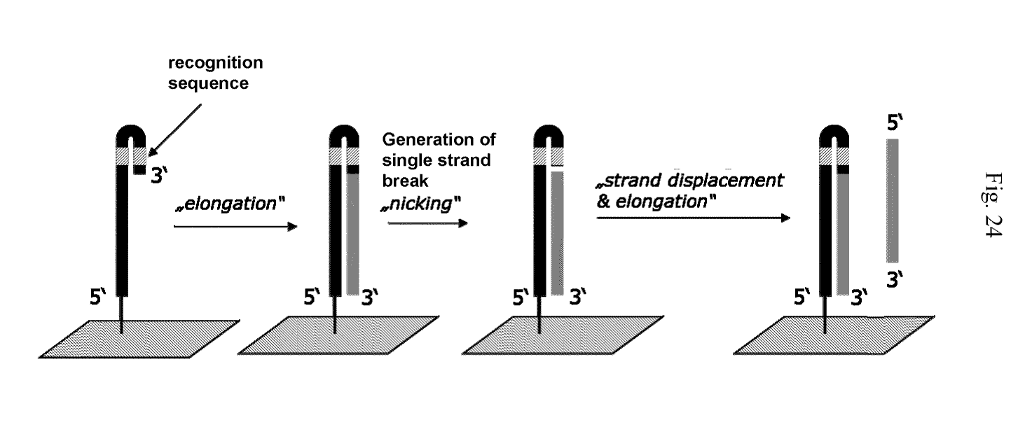

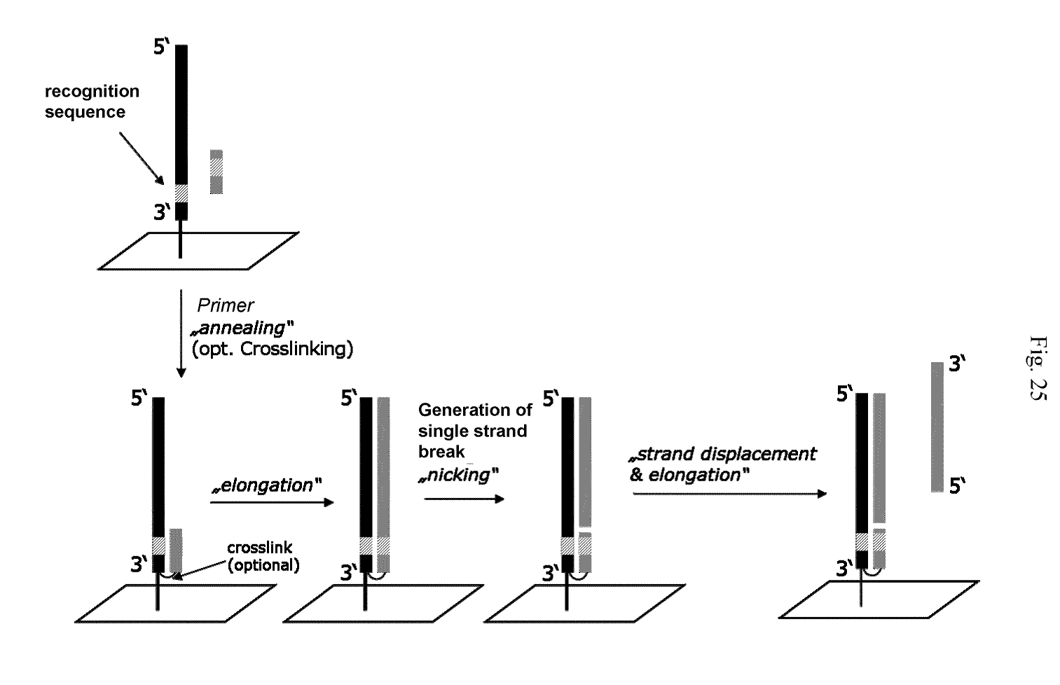

The probe molecules synthesized on the surface of the reaction support additionally contain a recognition sequence for nicking endonucleases known by a person skilled in the art. With sequential or simultaneous processing of the reaction support thus prepared with polymerases and nicking endonucleases, amplification of molecules can take place. After extension of the primer or of the hairpin by the nicking endonuclease, the newly synthesized strand, which was linked to the primer by the polymerase, can be cut at one point. Owing to the cut, the previously extended primer is again available as substrate for a polymerase. This is extended again, with the polymerase displacing the previously synthesized strand that has been detached from the primer, which is known by a person skilled in the art as strand displacement. Alternatively, the strand can also be displaced by a temperature change. This process can take place repeatedly, so that amplification of the molecules synthesized by the polymerase and cut off by the nicking endonuclease takes place. FIGS. 24 and 25 illustrate this preferred embodiment. Preferably, mixtures of polymerases and nicking endonucleases can be used, so that new synthesis and cutting off of the strands to be amplified can proceed in a mixture in a reaction support, without the need to change mixtures. Thermostable enzymes can also be used for this. Enzymes that can be used are for example the Klenow fragment of E. coli DNA polymerase I and mutants, such as 3′-5′-exonuclease deficient mutants, Bacillus stearothermophilus (Bst) DNA polymerase, Phi29 DNA polymerase, the endonucleases N.AlwI, N.BstNBI and others. The newly formed molecules are preferably DNA oligonucleotides or derivatized DNA oligonucleotides or DNA oligonucleotide analogs. These possess a length of preferably 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34.35, 36, 37, 38, 39, 40, 41, 42, 43, 44, 45, 46, 47, 48, 49, 50, 51, 52, 53, 54, 55, 56, 57, 58, 59, 60.61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 nucleotides.

6.22 Crosslinking of Hybridized Molecules with the Probe Molecules Synthesized in the Reaction Support

In another preferred embodiment, other molecules are bound and covalently linked to the molecules synthesized on the surface of the reaction support. Various methods, known by a person skilled in the art, are used for the crosslinking of biomolecules. Preferably nucleic acids are crosslinked with one another, i.e. the molecules synthesized on the surface of the reaction support are oligonucleotides or derivatives or analogs of DNA or RNA. These are hybridized sequence-specifically to oligonucleotides or derivatives or analogs of DNA or RNA introduced into the reaction support and are crosslinked with one another. For this it is possible for example to use crosslinking methods that are known by a person skilled in the art, based for example on a psoralen unit, or on aldehydes or ketones or radical-forming, or carbene-forming or nitrene-forming chemical groups.

### 6.23 Assembly of Short Probe Molecules to Longer Molecules Directly in the Reaction Support

In another preferred embodiment, molecules synthesized in the reaction support are linked to one another specifically, directly in the reaction support, to form longer molecules. The molecules that are to be linked can have been synthesized chemically on the surface of the reaction support or can have been produced by certain enzymatic methods. Preferably, directly after synthesis or through cleavage, these are in soluble form and are no longer bound to the surface. Methods such as PCR, primer extension or the methods described in 6.21 are preferably used for this. Various molecules can be linked together in a defined order. This can take place for example at mutually complementary, specific binding sites contained in the molecules. These binding sites bring about specific, initially noncovalent association of the molecules. Using particular enzymes, for example ligases or polymerases, these molecules can now be linked to one another covalently. The length of the molecules to be linked can be 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34.35, 36, 37, 38, 39, 40, 41, 42, 43, 44, 45, 46, 47, 48, 49, 50, 51, 52, 53, 54, 55, 56, 57, 58, 59, 60.61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 nucleotides. Preferably oligonucleotides or derivatives or analogs of DNA or RNA are linked. This results in long fragments ranging from hundreds to thousands of nucleotides.

6.24 Control of the Assembly of Short Probe Molecules to Longer Molecules Directly in the Reaction Support Based on the Quantitative Proportions of Certain Probe Molecules and their Location in the Reaction Support

In another preferred embodiment, molecules synthesized in the reaction support are specifically linked together to longer molecules directly in the reaction support. The linkage, taking place as in 6.23, is controlled on the basis of certain properties of the reaction support. Preferably, the amount of substance of individual molecular species relative to the amount of substance of other molecules can be varied in a manner that is favorable to particular positioning on particular sites in the later, covalently linked target molecule. Especially preferably, the order of linkage and the position of the individual molecules in the later, covalently linked target molecule can also be controlled, by synthesizing the individual molecules at addressable locations on the surface of the reaction support in a manner such that their spatial position on the surface relative to one another promotes directed, specific linkage. For example, molecules that are to be linked together directly in the later, covalently linked target molecule and so should be next to one another can also be synthesized at adjacent locations of the reaction support. Based on physical effects, such as different rates of diffusion of the individual molecules to one another, controlled for example by different wavelengths, the timing of the collisions and hence of the linkage of individual molecules can also be controlled. For example, individual populations can be synthesized very close together in island-like groups and after detachment or after preparing soluble copies that are no longer linked to the surface, are preferably linked together in such a way that the diffusion paths are short and the molecules quickly collide. Within the islands, this results in longer, linked molecules composed of relatively few individual molecules, so that the complexity of association and linkage remains low. If several such islands are farther apart than the molecules within an island and if for example the linkage is complete and quicker than the diffusion of the nonlinked individual molecules from island to island, the molecules prelinked within an island can in each case diffuse to the next island and encounter the prelinked molecules there, after which they become linked to them. As a result, the number of molecules and the complexity during the individual linkings remain low and larger and larger fragments can be built up. The strength of this regulatory mechanism can be further controlled by using viscous solvents.

6.25 Use of the Reaction Support for the Detection of microRNAs and Other Small RNAs

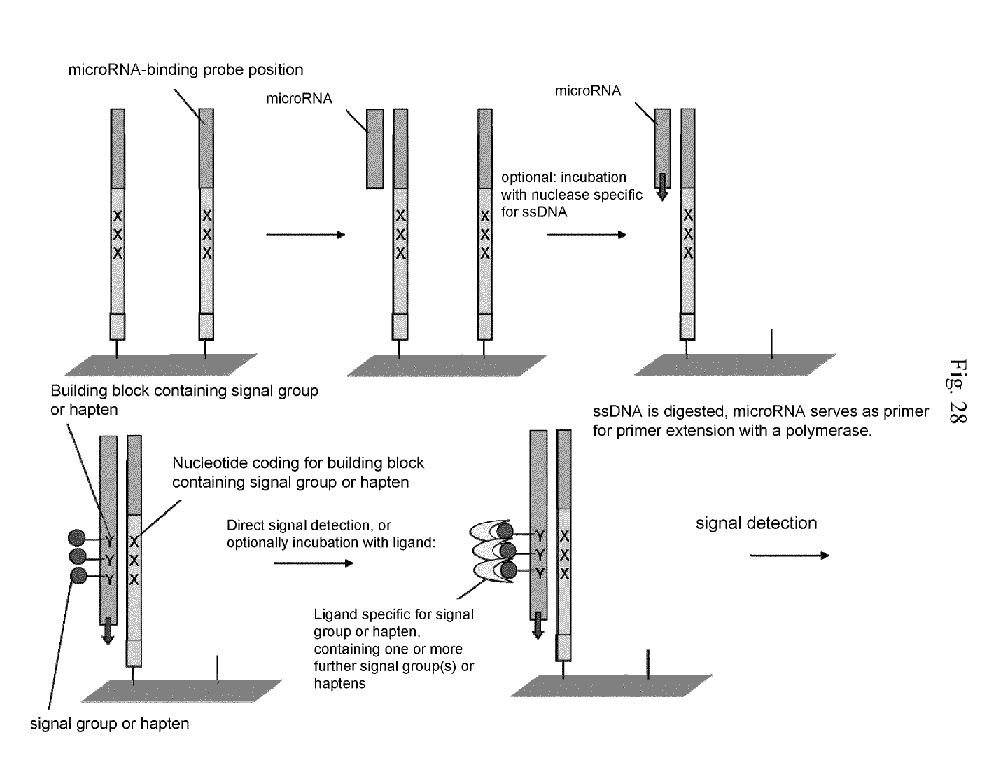

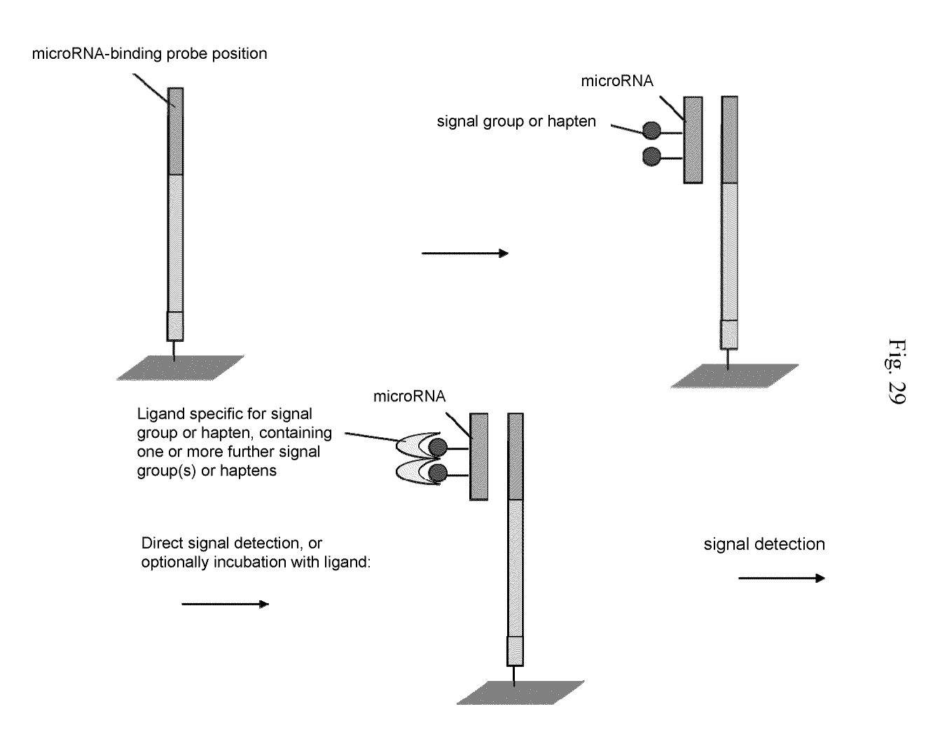

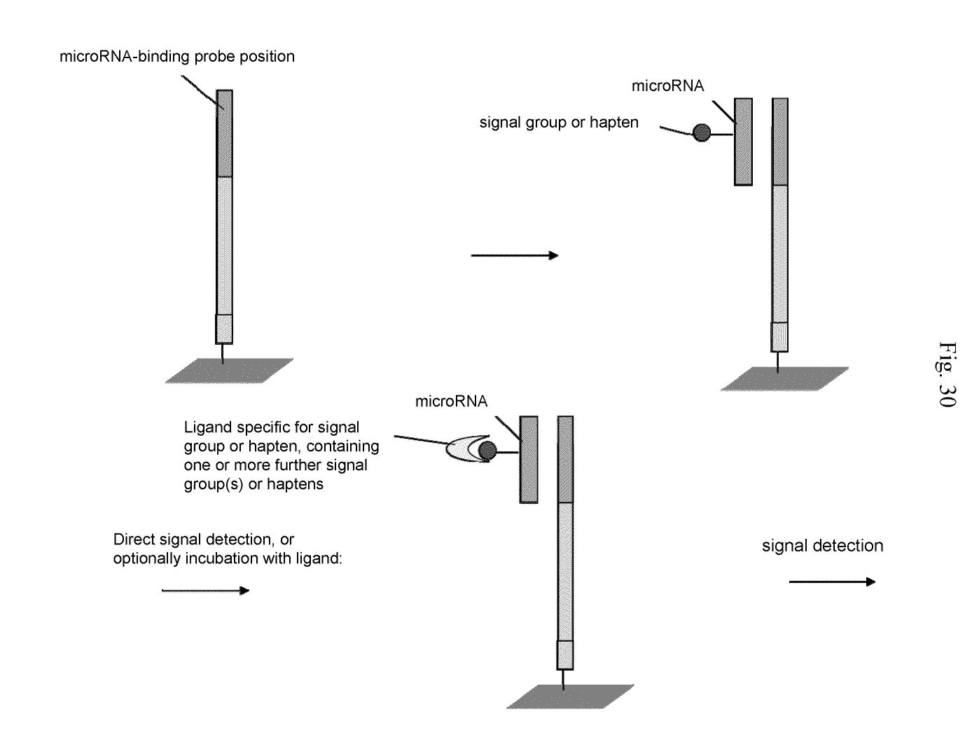

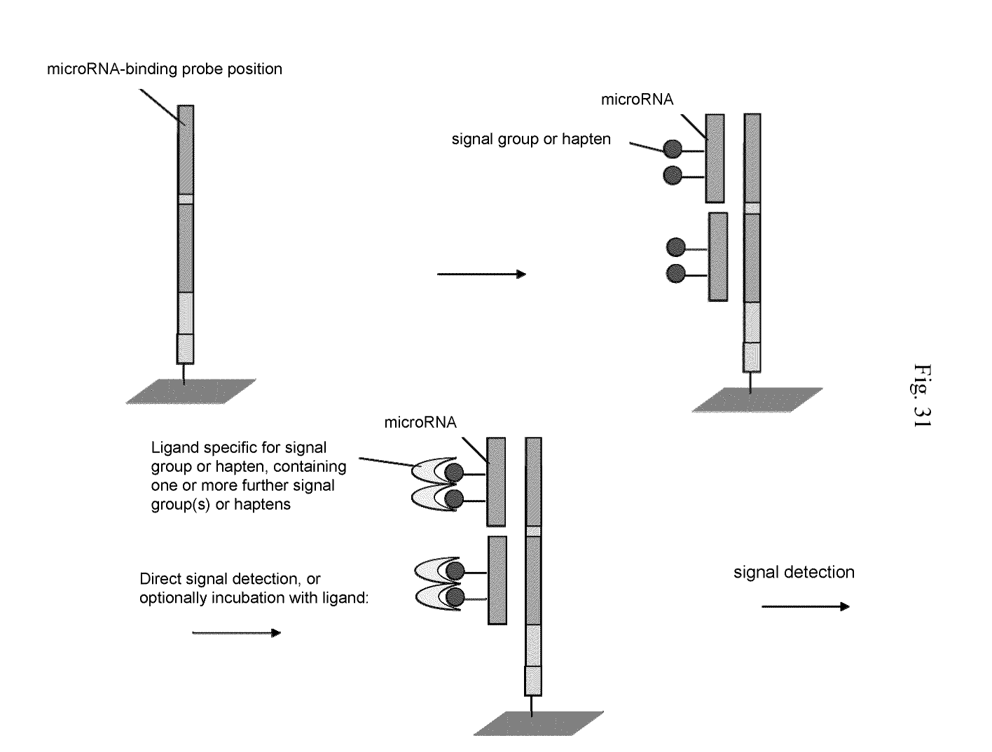

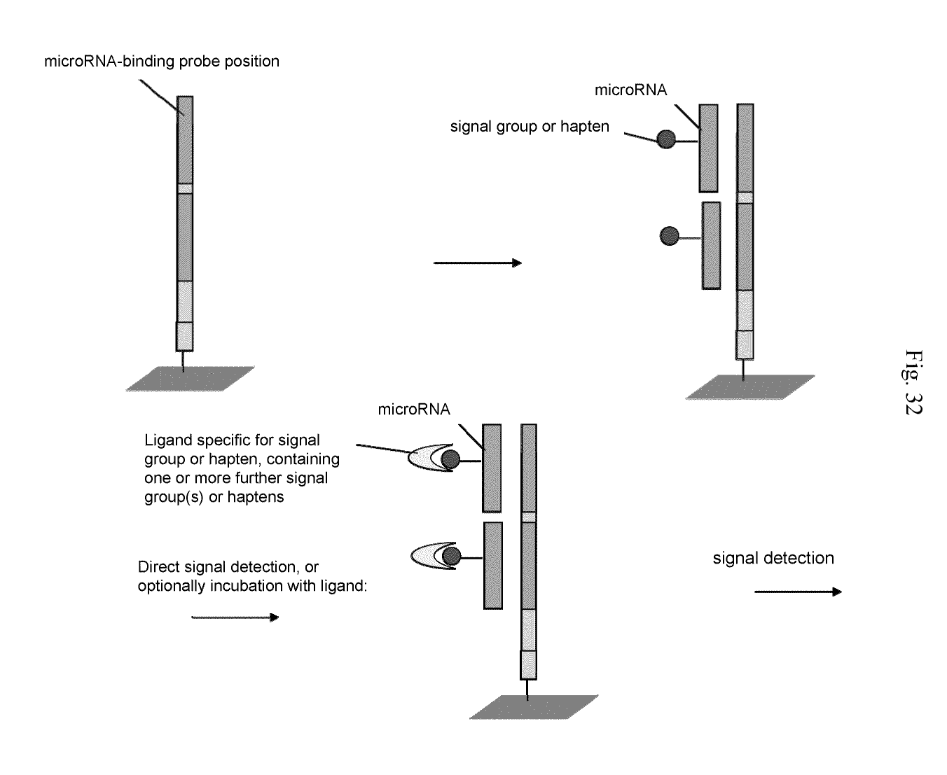

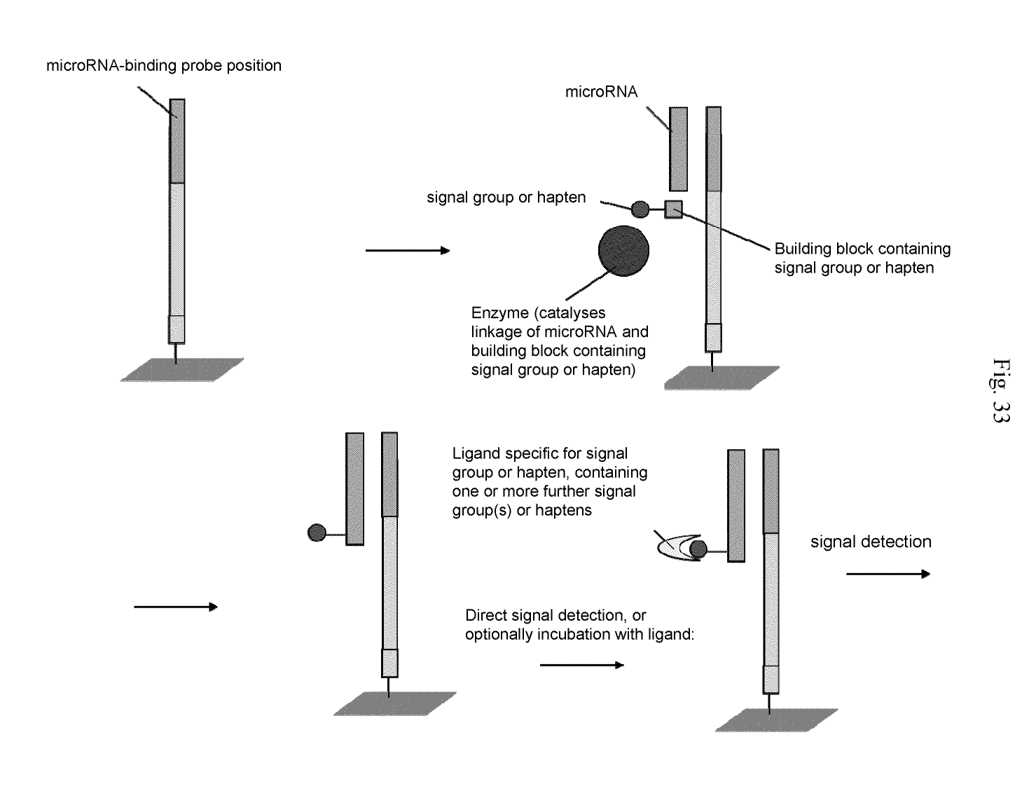

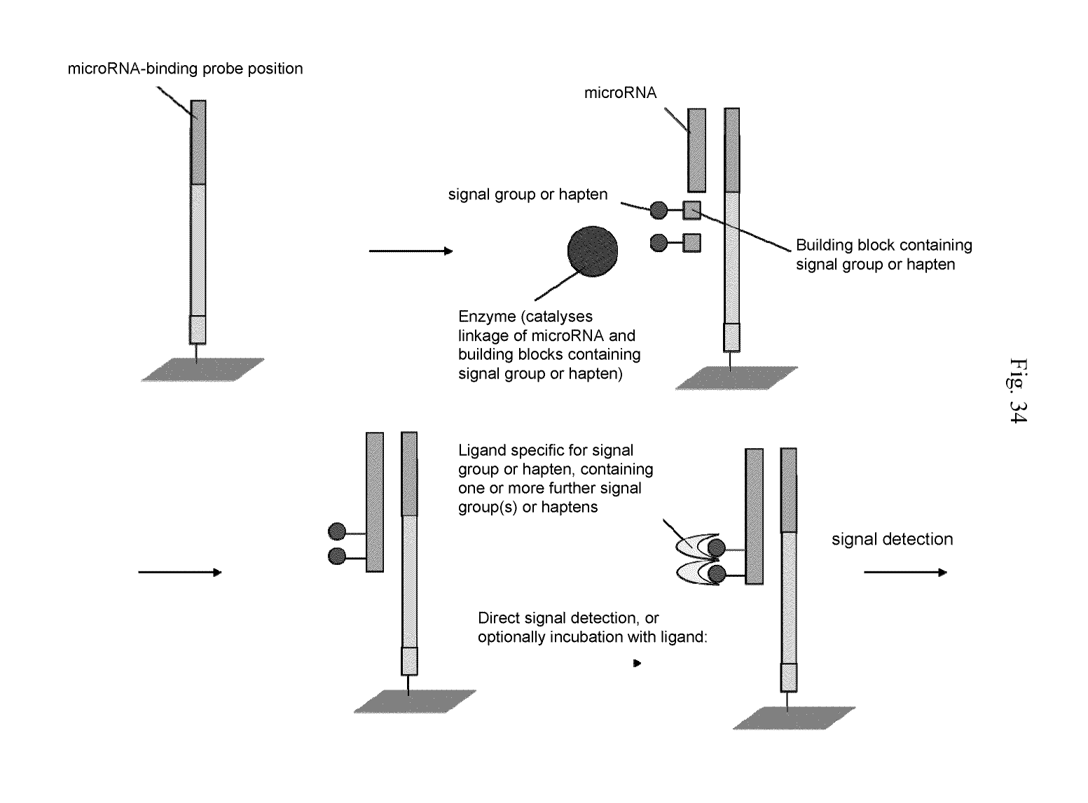

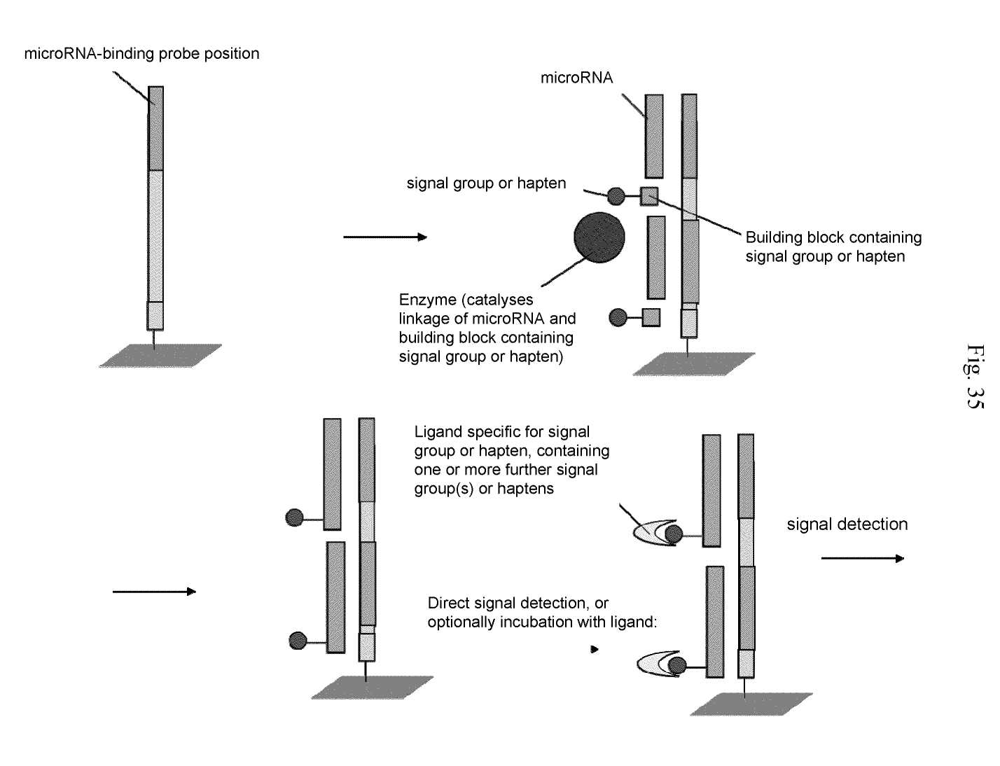

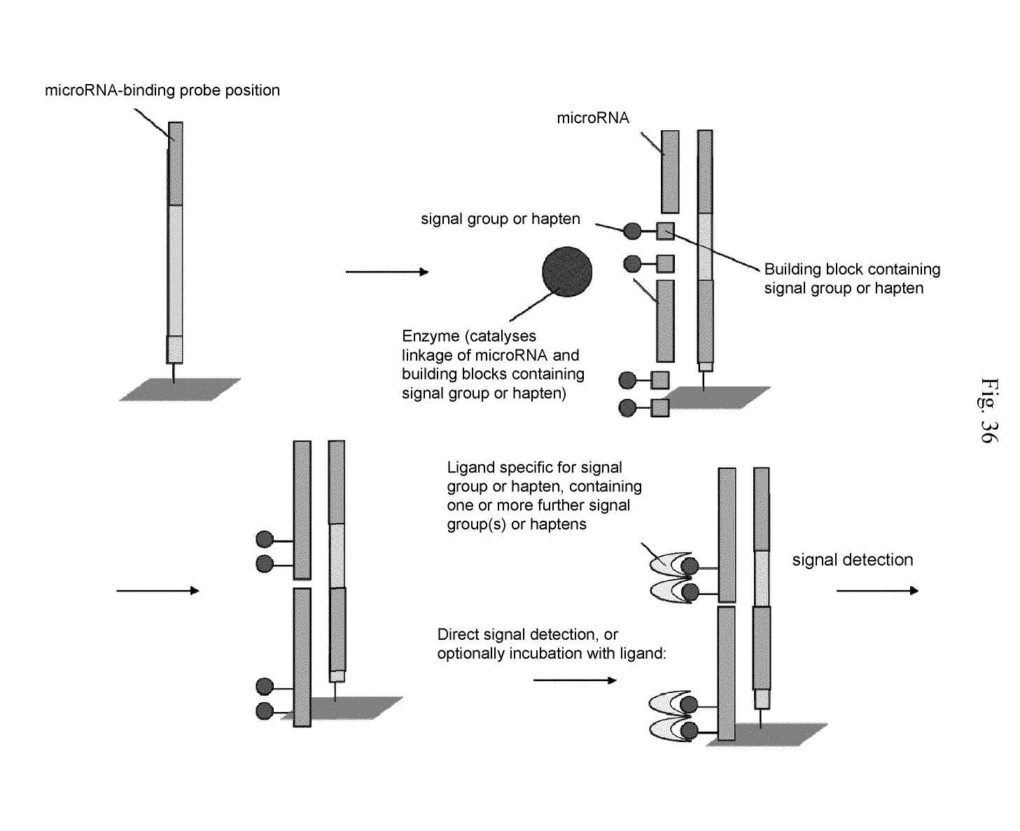

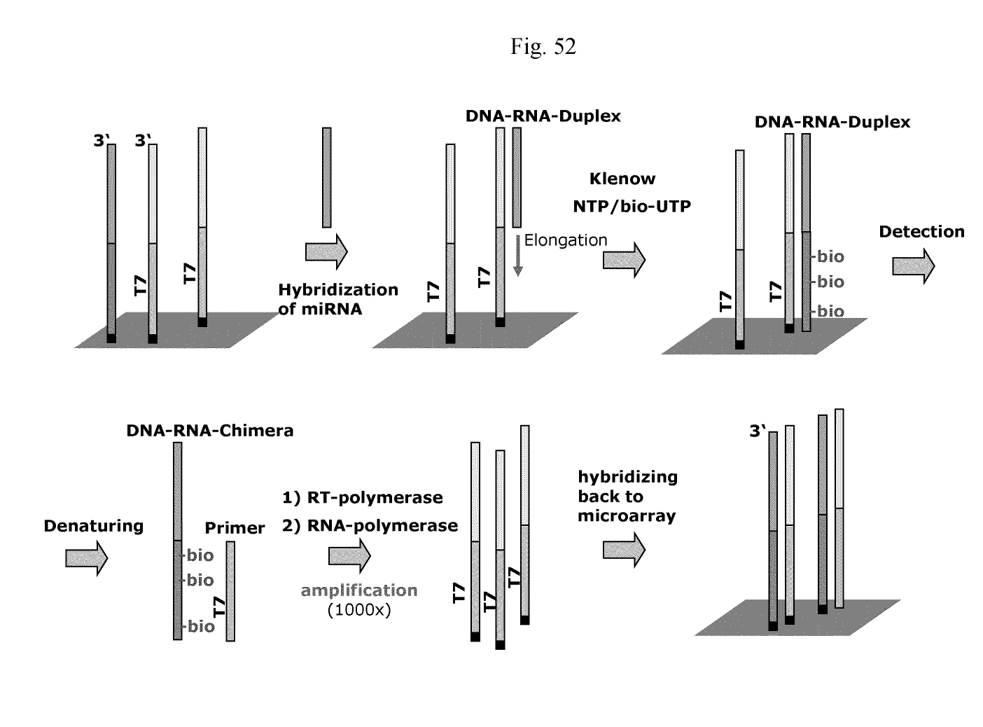

In another preferred embodiment, the receptors synthesized on the surface of the reaction support are used for detecting microRNAs (also called “miRNAs”) and other small RNAs in sample mixtures. FIGS. 28 to 36 and 44 to 53 illustrate this preferred embodiment. The receptors synthesized on the surface of the reaction support can preferably be linked to the surface via the 5′ or the 3′ end, so that the free end of the receptor is preferably either a 3′ or a 5′ end. Linkage can be direct or via a linker. The receptors preferably contain one or more binding sites, which specifically bind particular microRNAs or other small RNAs, i.e. hybridize to them. Preferably there are 1, 2, 3, 4, 5, 6, 7, 8, 9 or 10 binding sites, especially preferably 1, 2 or 3 binding sites. The binding sites can border directly on one another or can be separated by small intermediate regions of a predetermined length. These intermediate regions can have a length of 1, 2, 3, 4, 5, 6, 7, 8, 9, 10, 11, 12, 13, 14, 15, 16, 17, 18, 19 or 20 nucleotides.

The molecules synthesized on the surface of the reaction support are preferably oligonucleotides or derivatives or analogs of DNA or RNA with a length of preferably 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 individual building blocks such as nucleotides or derivatives or analogs of nucleotides. The microRNAs or other small RNAs can therefore be bound specifically and so can be detected in complex sample mixtures by detection techniques that are known by a person skilled in the art from microarray technology. Prior to introduction into the reaction support or also directly in the reaction support before, during or after binding to the molecules synthesized on the surface of the reaction support, the molecules to be detected can be linked to signal-emitting groups or haptens, making it possible for them to be detected, preferably from an optical signal. A large number of methods, known by a person skilled in the art, are available for marking (labeling), e.g. by direct labeling with biotin or fluorophors or indirectly during cDNA synthesis or amplification. Both chemical and enzymatic methods are known for this, e.g. based on cisplatin compounds, periodate-hydrazine labeling, T4 RNA ligase, poly(A) polymerase or coupling to aminomodified RNAs. The signal-emitting groups can in particular be fluorescent groups or fluorophors or FRET quenchers or FRET acceptor groups or luminescent groups, known by a person skilled in the art. These can be introduced directly or linked as groups with other chemical units, e.g. dendrimers, or with ligands, which bind hapten groups attached to the RNA beforehand. FIGS. 28-36 and 44 to 53 illustrate these preferred embodiments of the present invention.

Preferably, signal amplification can also be used, for example the introduction of a hapten such as biotin, the subsequent binding of a conjugate of streptavidin and a fluorophor or several fluorophors. Optionally, following that, another ligand can be bound, which in its turn is linked to one or more haptens or fluorophors, so that binding of a larger number of haptens or fluorophors occurs. Next, another ligand can bind, which in its turn is linked to one or more haptens or fluorophors, so that binding of a larger number of haptens or fluorophors occurs. This process can take place several times, preferably 1, 2, 3, 4, 5, 6, 7, 8, 9 or 10 times. Antibodies are preferably used as ligands; preferably biotin or digoxigenin as haptens. Preferably, oligonucleotides can also be used as primers in rolling-circle amplification, known by a person skilled in the art, and are linked to streptavidin. The streptavidin-biotin conjugate can have bound previously to biotin units, which were previously linked to hybrids of probe molecules and sample molecules.

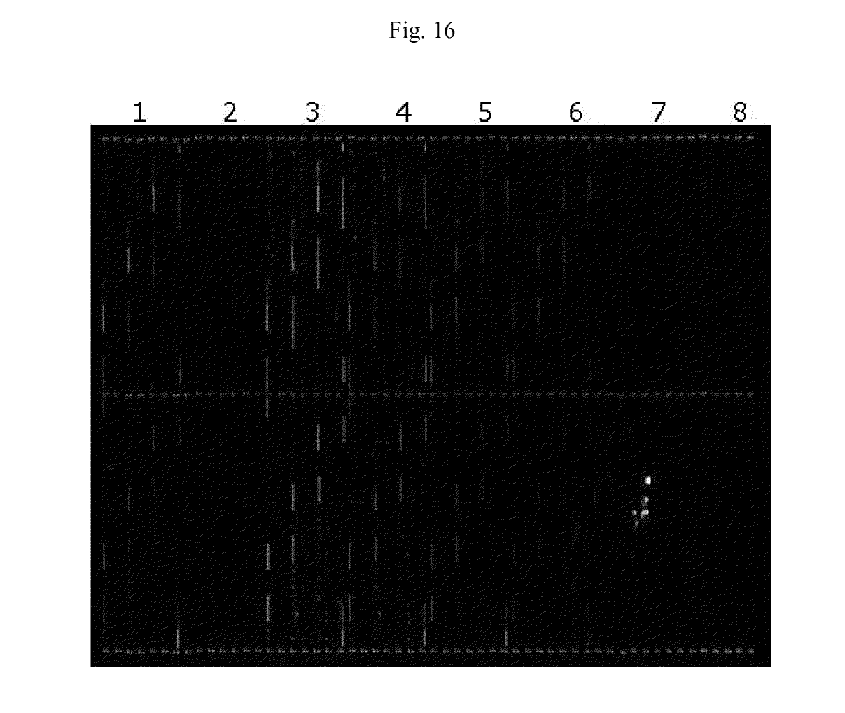

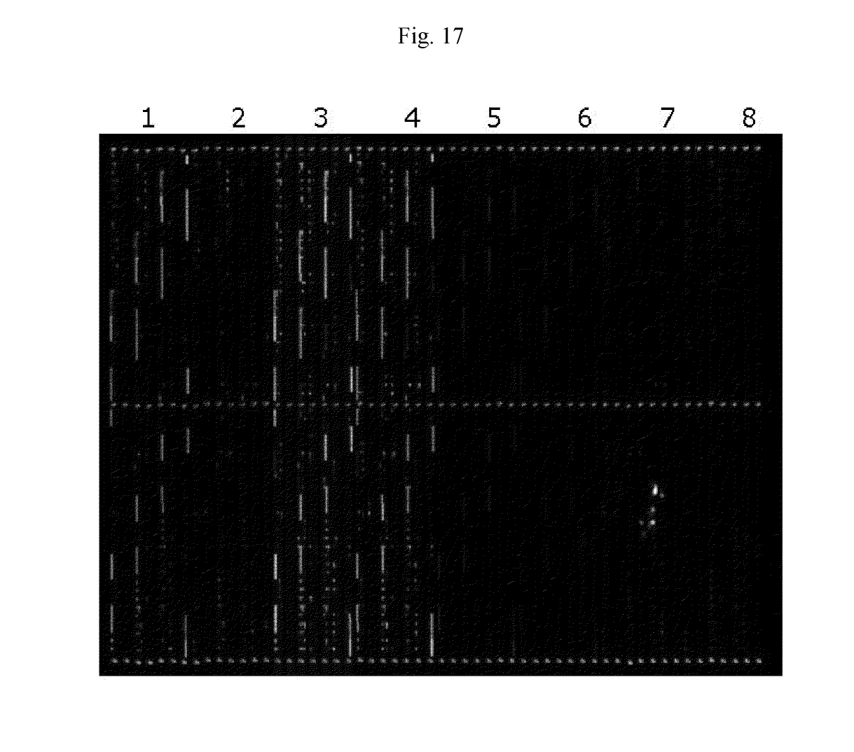

FIGS. 16 and 17 show data from successful experiments for this preferred embodiment.

In also preferred embodiments, microRNAs or other analytes on the surface of the reaction support are amplified. This amplification can be a single-strand amplification or a double-strand amplification. In further preferred embodiments the microRNA or some other analyte is detected before, during or after the amplification as described above by the insertion of labeled building blocks. In further preferred embodiments the microRNA or some other analyte is detected with DNA-intercalators, molecular beacons, Taqman probes and other methods known by a person skilled in the art.

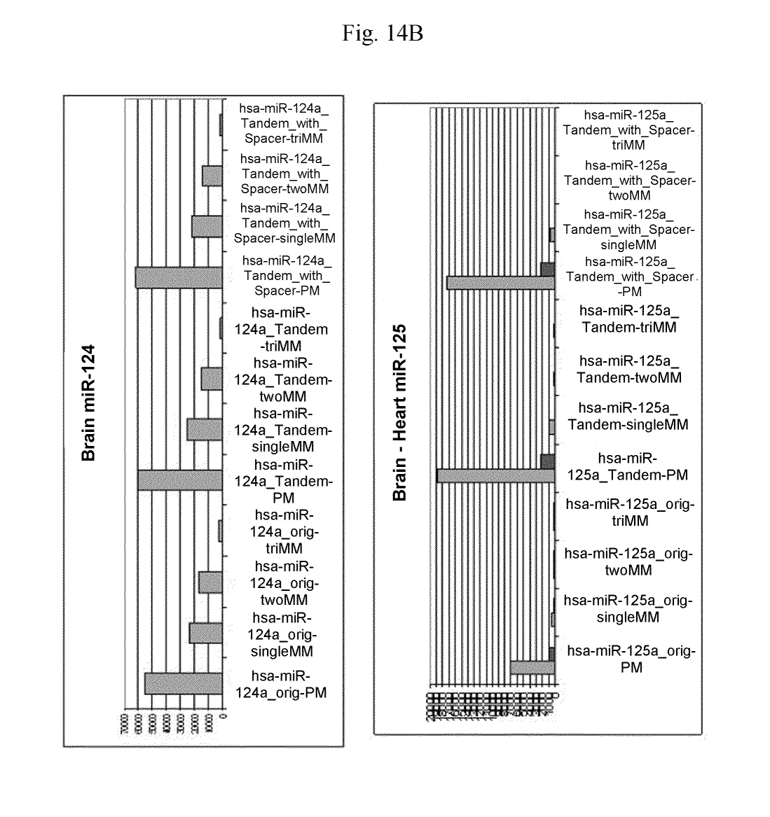

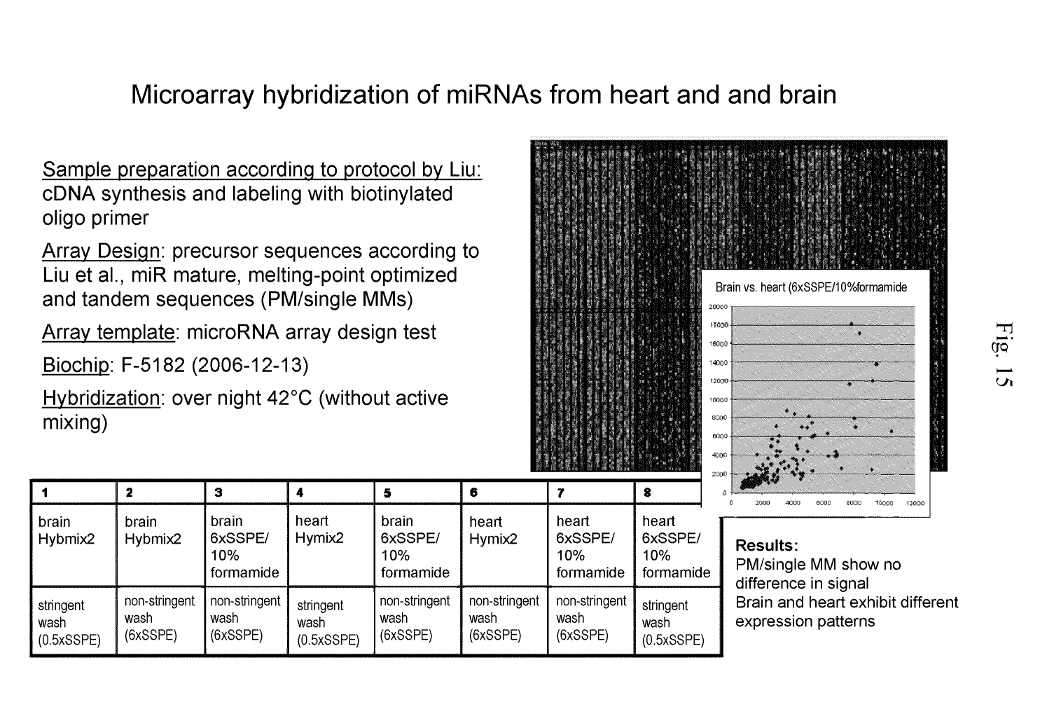

The molecules synthesized on the surface of the reaction support and used for the binding of particular miRNAs or other small RNAs can, with respect to their desired binding site or sites, be completely or only partially complementary to the sequence of the RNA that is to be bound. For example 1, 2, 3, 4, 5, 6, 7, 8, 9 or 10 individual bases can be noncomplementary to the sequence of the RNA that is to be bound. FIG. 14 shows data from successful experiments, in which microRNAs in complex sample mixtures of various tissues were detected in the manner described. Molecules with 1 or 2 binding sites were used for binding the microRNAs, which bordered on one another directly or were separated from one another by intermediate regions and were either completely complementary to the sequence of the RNA that is to be bound, or noncomplementary—at 1, 2, or 3 nucleotide positions—to the sequence of the RNA that is to be bound. FIG. 15 presents detailed optimization results with respect to temperature and buffer conditions for the specific detection of a large number of microRNAs from a complex sample mixture.

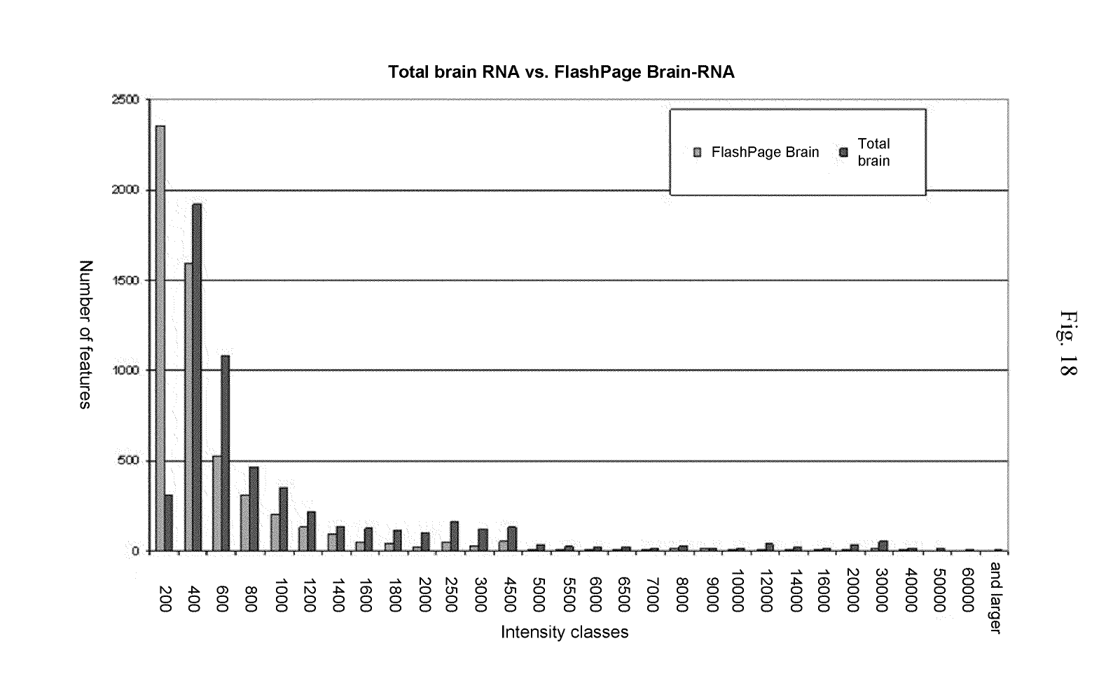

The type of sample mixture can vary: unpurified or completely or partially purified extracts from cells or tissues can be used. This can for example be total nucleic acid purification, total RNA purification or special purifications, which make enrichment of small RNAs, e.g. microRNAs, possible. Numerous methods for this are known by a person skilled in the art. FIG. 18 shows data from successful experiments, in which microRNAs from various tissues were detected in the manner described. The complexity of the sample mixture varied: complete RNA extracts were used, or small RNAs were enriched beforehand using a special purification process.

The RNAs to be detected can also be processed enzymatically in a particular manner directly in the reaction support, prior to detection. Lengthening of the bound RNAs by a polymerase is especially preferred, as in a primer extension known by a person skilled in the art, wherein one or more signal-emitting groups or haptens can be linked simultaneously to the RNA. This can preferably take place by insertion of nucleotides modified with signal-emitting groups or with haptens by a polymerase. The type and number of the nucleotides modified with signal-emitting groups or haptens can be determined by the composition of the probe molecule. This can preferably be effected with a template sequence, which genetically encodes the type and number of incorporated building blocks by the presence of particular nucleotides. Preferably, nucleases can also be used in the reaction support, which selectively cleave or hydrolyze or digest the molecules synthesized on the surface of the reaction support, that are not bound to an RNA, or selectively cleave or hydrolyze or digest the molecules synthesized on the surface of the reaction support, which have bound to an RNA. FIG. 28 illustrates this preferred embodiment of the invention. In an especially preferred embodiment, desired microRNAs and other small RNAs present in the sample mixture to be investigated are amplified specifically. Owing to the capability of producing a large number of different sequences in the reaction support and using these as primers, a large number of amplification reactions individually adapted to the respective RNAs to be amplified can be carried out in parallel. The full spectrum of amplification reactions that are known by a person skilled in the art is available. In particular the methods described in 6.1, 6.5, 6.6, 6.9, 6.15, 6.16, 6.18, 6.19, 6.20 and 6.21 can be used. The RNAs to be investigated can function either as template or as primer or both and/or can be linked to universal sequences before amplification.

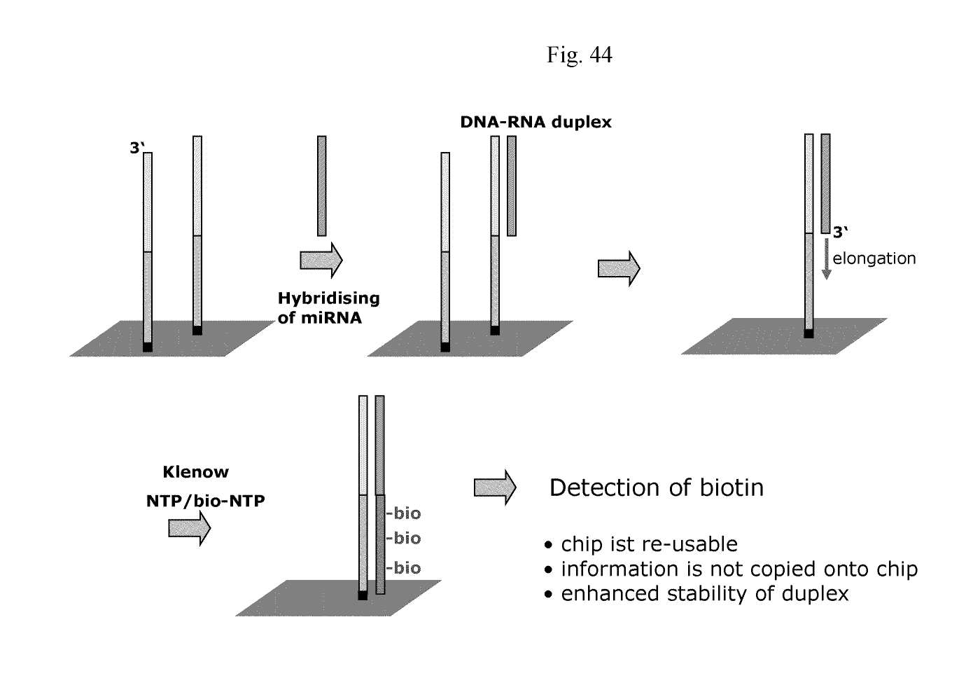

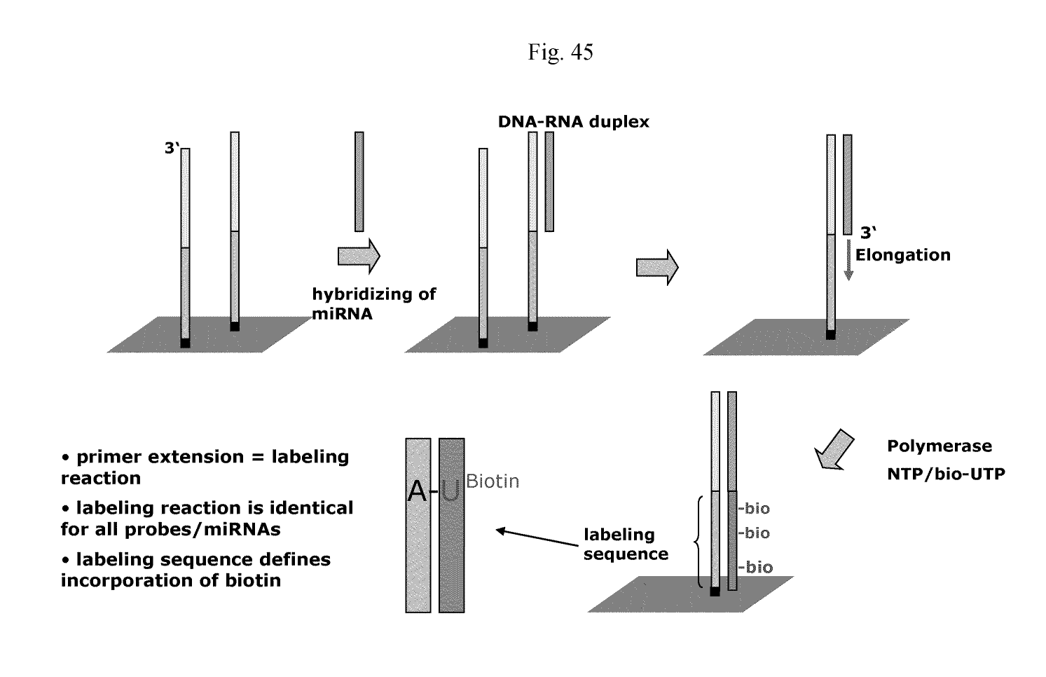

FIGS. 44 and 45 show a preferred embodiment in which the receptors are linked via the 5′ end to the surface of the support. The hybridization region with the microRNA is positioned on the free 3′ end of the receptor. The hybridization region is preferably arranged so that after the hybridization of the microRNA, the receptor still has at least 1, preferably 2, 3, 4, 5, 6, 7, 8, 9, 10, 11, 12, 13, 14, 15, 16, 17, 18, 19, 20, 21, 22, 23, 24, 25 or more nonhybridized receptor building blocks at the 5′ end. This nonhybridized 5′ region of the receptor preferably has 1, 2, 3, 4, 5, 6, 7 or more building blocks, which can serve as templates for building blocks containing signal groups, e.g. adenines when using biotin-marked uridinen as building blocks containing hapten groups. The free 3′ end of the hybridized microRNA serves as primer for the subsequent amplification. The amplification can consist of only single extension, preferably by a DNA-dependent DNA polymerase, for example Klenow fragment. Preferably, during this amplification, building blocks containing signal groups or haptens, preferably nucleotide building blocks, are incorporated. The amplification preferably comprises the covalent linkage of 1, 2, 3, 4, 5, 6, 7, 8, 9, 10, 11, 12, 13, 14, 15, 16, 17, 18, 19, 20, 21, 22, 23, 24, 25 or more building blocks. The result of this amplification, when using deoxynucleotide building blocks, is an RNA-DANN hybrid.

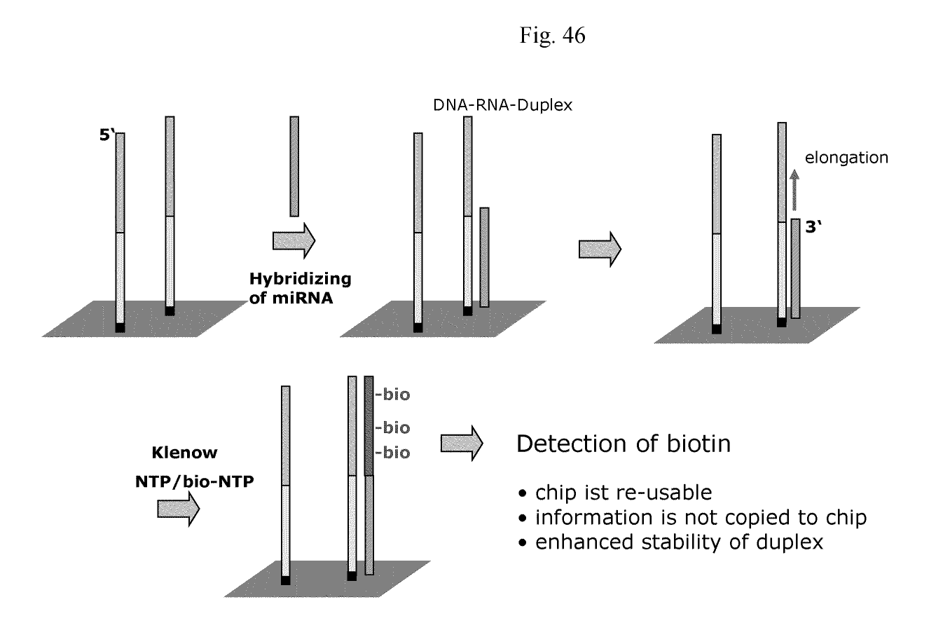

Alternatively, as shown in FIG. 46, the receptor can be linked to the surface of the support via the 3′ end. In this case the hybridization region with the microRNA is preferably located at the 3′ end of the receptor and is preferably arranged in such a way that, after hybridization of the microRNA, the receptor still has at least 1, preferably 2, 3, 4, 5, 6, 7, 8, 9, 10, 11, 12, 13, 14, 15, 16, 17, 18, 19, 20, 21, 22, 23, 24, 25 or more nonhybridized receptor building blocks at the 5′ end. Amplification preferably takes place starting from the 3′ end of the microRNA up to the end of the receptor. The nonhybridized 5′ region of the receptor is preferably selected as described previously.

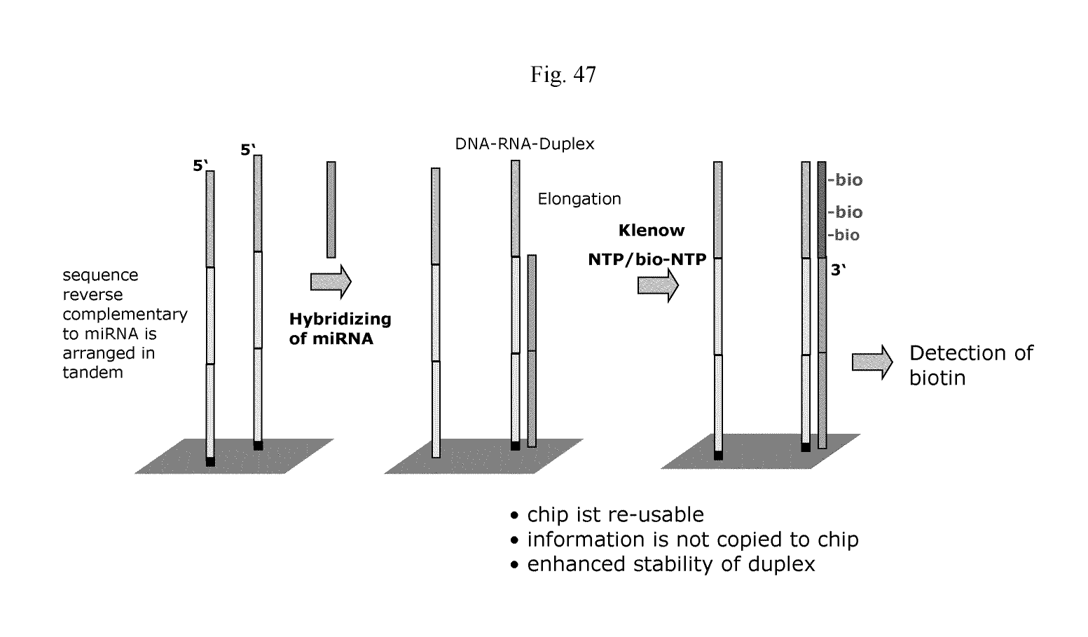

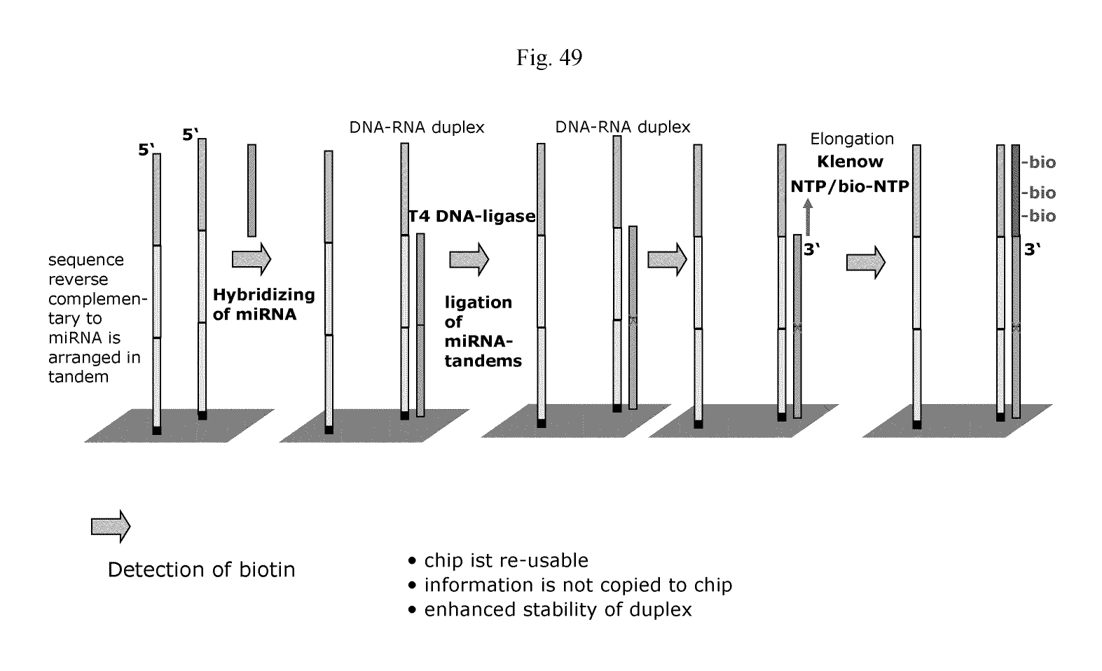

To increase the stability of the DNA-RNA duplex, the receptor can have 2, 3, 4, 5, 6, 7 or more, preferably 2 hybridization regions that are reverse-complementary to the microRNAs to be detected. Preferably these regions are arranged in such a way that there are no unhybridized receptor building blocks between the hybridized microRNAs. The receptors can be linked to the surface of the support via the 3′ end (see FIGS. 47 and 49) or can be linked to the surface of the support with the 5′ end. In a preferred embodiment the two, three, four or more microRNAs hybridized to the receptor are covalently linked by a suitable ligase, for example RNA ligase or T4 DNA ligase (see FIG. 49). In this embodiment, the amplification preferably takes place after the ligation of the microRNAs by a suitable polymerase, preferably a DNA-dependent DNA polymerase, for example Klenow fragment.

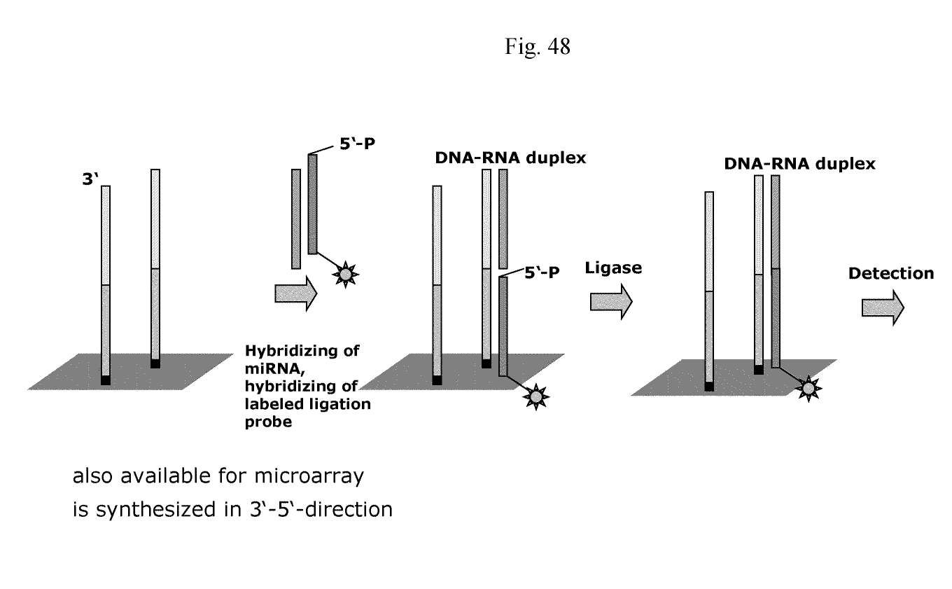

In another embodiment in which the receptor is linked either via the 5′ or 3′ end to the surface of the support, in addition to the analyte to be detected, e.g. the microRNA, one or two ligation probes are hybridized to the receptor either together with the analyte or before or after the hybridization of the analyte (see FIG. 48). In the case of simultaneous addition, the ligation probe(s) is/are for example added to the sample to be analyzed and mixed with it. The ligation probe(s) has/have a sequence that allows it/them to hybridize to the receptor 3′ and/or 5′ of the microRNA to be detected. Preferably the receptor sequence and the sequence of the ligation probes are selected in such a way that after hybridization of the microRNA and the ligation probe(s), no unhybridized receptor building blocks are arranged between the respective free 3′ and 5′ ends. The ligation probe has at least one signal-emitting group or detectable group, for example a hapten and at least one group that can be linked by a ligase to a free 3′-OH group or 5′-phosphate group of the microRNA, for example an OH group or a monophosphate group. Ligation preferably takes place in a separate step subsequent to the hybridization with a suitable ligase, for example RNA ligase or T4 DNA ligase. If using a heat-resistant ligase, however, hybridization and ligation can also take place in one step, by which an acceleration of the detection reaction can be achieved. Detection of the group(s) contained in the ligation probe preferably takes place after washing the surface of the support, with a stringency that is preferably selected such that hybridized, nonligated microRNAs are washed away from the surface of the support.

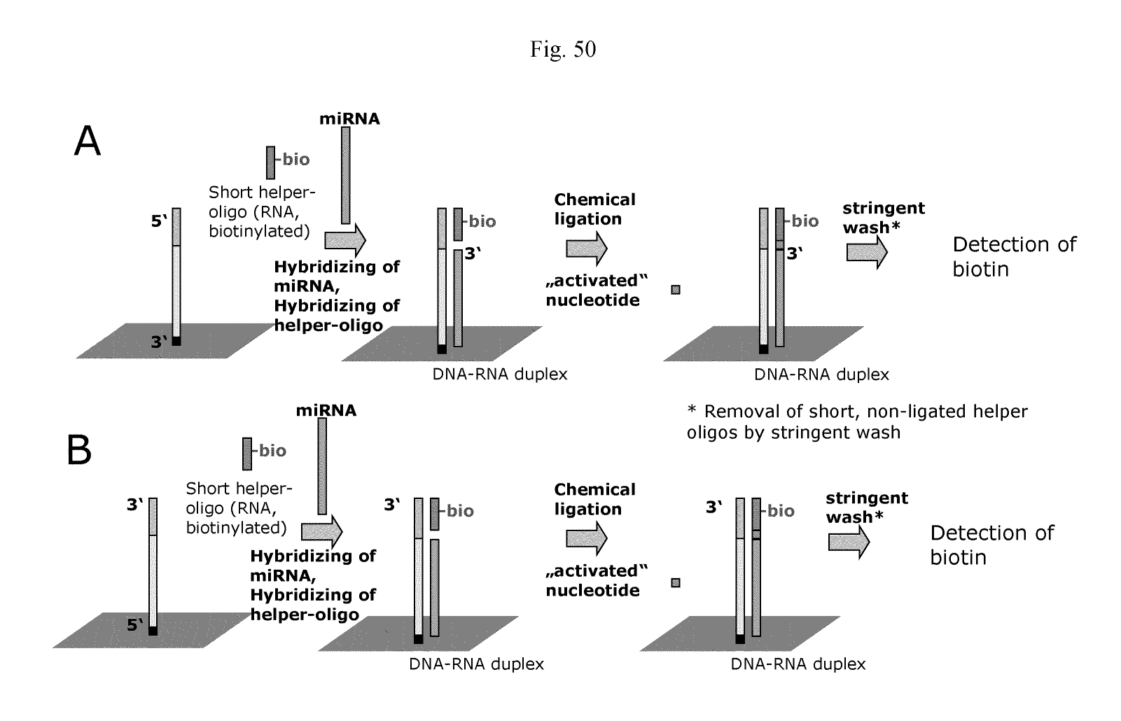

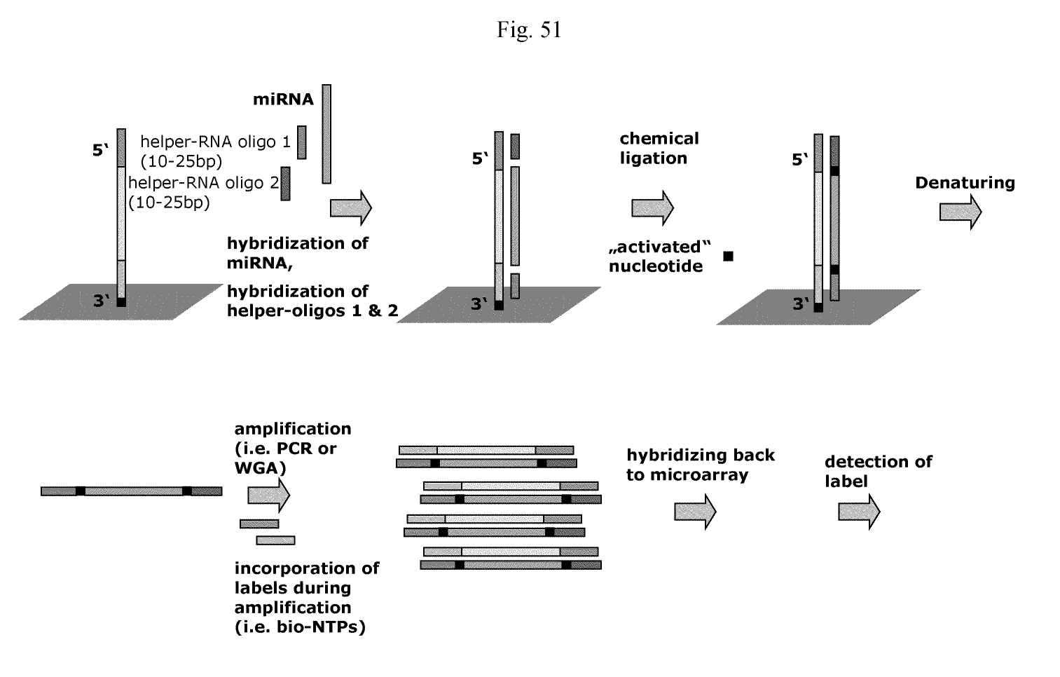

In a variant of the embodiment described in FIG. 48, instead of an enzymatic linkage of the 3′ end of the analyte, e.g. microRNA, a chemical linkage is achieved with suitable activated nucleotides. Suitable chemical linkages are described for example in international patent application WO 2006/063717 (the contents of this application, with respect to chemical ligation, are fully incorporated by reference in the present application). This linkage can take place both at the 3′ and at the 5′ end of the analyte, e.g. the microRNA. It is possible to use either only an activated nucleotide or an oligonucleotide with an activated nucleotide arranged on its 3′ or 5′ end (see FIGS. 50 and 51). When an activated oligonucleotide is linked to the analyte, in particular a microRNA, the sequence is selected in such a way that it is complementary to the receptor sequence, which is arranged 5′ and/or 3′ next to the receptor sequence to which the analyte is hybridized. In this case a sequence-specific hybridization of the oligonucleotide takes place first, followed by a chemical linkage to the hybridized microRNA. Preferably the oligonucleotide and/or the activated nucleotide comprise a signal-emitting and/or a detectable group, for example hapten-containing groups, in particular biotin. In an especially preferred embodiment, one or two short helper oligonucleotides are hybridized to the receptor before, after or together with the analyte, in particular the microRNA. The helper oligonucleotide is preferably 3, 4, 5, 6, 7, 8, 9, 10, 11, 12, 13, 14, 15, 16, 17, 18, 19 or 20 nucleotides long and has a sequence that allows the helper oligonucleotides to hybridize 3′ and/or 5′ next to the analyte, in particular the microRNA. Hybridization of the one or the two helper oligonucleotides and of the analyte is followed by chemical ligation using an activated nucleotide or oligonucleotide. When using an activated nucleotide, the sequence of the helper oligonucleotide is selected in such a way that after the hybridization an unhybridized receptor nucleotide is arranged between the respective ends of the helper oligonucleotide and of the analyte. When using an activated oligonucleotide, the sequences of the helper oligonucleotide and of the receptor are selected in such a way that after the hybridization of the analyte and of the helper oligonucleotide to the receptor, the number of unhybridized receptor nucleotides between the 3′ or 5′ end of the helper oligonucleotide and the 5′ or 3′ end of the analyte corresponds to the number of nucleotide building blocks of the activated oligonucleotide. When using a helper oligonucleotide, this can contain signal-emitting and/or detectable groups, for example hapten-containing groups, in particular biotin. In this case the activated oligonucleotide can additionally contain a signal-emitting and/or detectable group. For example, helper oligonucleotide and activated nucleotide or activated oligonucleotide can in each case contain the groups of a FRET pair, so that a FRET pair is only present after chemical linkage of the helper oligos to the analyte has taken place. The step of chemical ligation, which in some embodiments can take place together with hybridization, is followed by the detection step. Preferably, before detection, the surface of the support is washed under conditions of stringency that are preferably selected so that hybridized, nonligated helper oligonucleotides are washed away from the surface of the support.

In a preferred embodiment, the first amplification step or ligation step in the previously described method can be followed by one or more further amplification steps. For this purpose, the first amplificate (see FIGS. 45 to 47) or the ligation product produced by chemical (see FIG. 50 or 51) or enzymatic ligation (see FIGS. 48 and 49) is removed from the receptor (denatured) by heating. By adding one or two oligonucleotides (primers) that hybridize to the first amplificate or ligation product, the amplificate or the ligation product can be amplified linearly (using a primer) or exponentially (using two primers). The sequence(s) of the primer or primers is/are preferably selected in such a way that they hybridize to the part added on in the first amplification or by the ligation of the analyte, in particular the microRNA. In some embodiments the primer or primers are selected in such a way that it or they both hybridize to the analyte and to the part added on in the first amplification or the ligated part. This can ensure higher specificity for amplificates of particular analytes. If the first amplificates or ligation products of different receptors are to be amplified simultaneously, it is preferable that the primer or primers hybridize exclusively to the sequences that were added on by ligation or the first amplification of the various analytes, as in this way one amplification reaction amplifies all various first amplificates or ligation products. In preferred embodiments, in this amplification reaction the primers and/or the nucleotide building blocks can be labeled with signal-emitting or detectable groups. In another step, the amplification can be followed by a back-hybridization to the receptors on the surface of the support. Detection of the amplificates takes place optionally after a stringent washing step on the surface of the support.

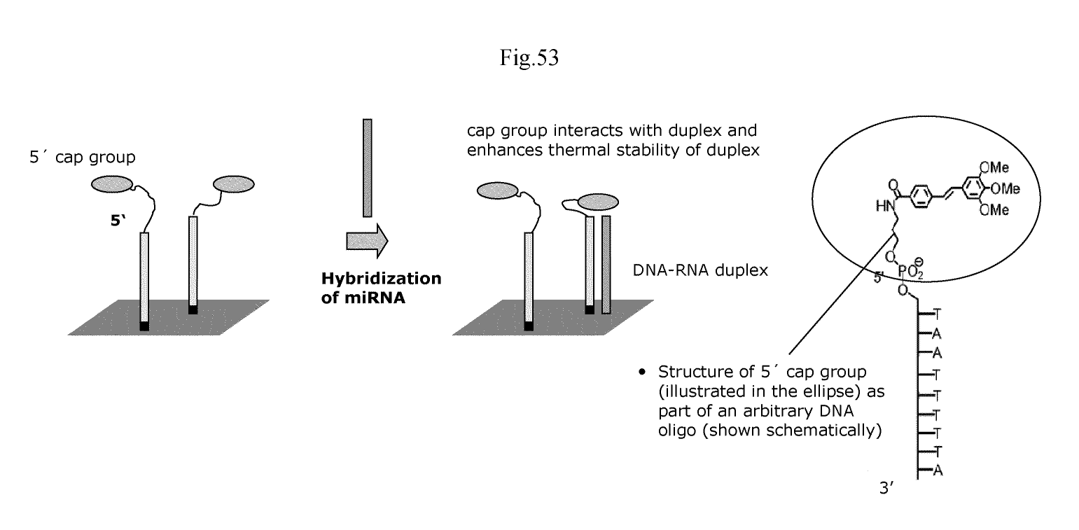

In another preferred embodiment a Cap group is located on the free end of the receptor, preferably on the free 5′ end of the receptor (see FIG. 53). In the case of hybridization of an analyte, preferably a microRNA, near or at the free end of the receptor. “Cap groups” in the sense of the invention interact with the duplex formed by hybridization and stabilize it. Suitable stabilizing Cap groups are known by a person skilled in the art and comprise for example substituted or unsubstituted bicyclic or tricyclic aromatics or heteroaromatics and stilbenes, in particular trans-stilbene (e.g. trans-1,2,3-trimethoxy-5-stilbene). Especially preferred Cap groups are capable of intercalating into the duplex formed and, through the intercalation, bring about stabilization of the duplex, i.e. Cap groups that are especially preferred are DNA-intercalators.

Overall, use of the molecular-biological processing equipment described for the detection, quantification and characterization of microRNAs and other small RNAs represents a considerable improvement over the prior art. As the number of naturally occurring small RNAs such as microRNAs is still unknown and new RNAs are being discovered all the time in very short periods, adaptation of analytical techniques to the conditions obtaining in each case, i.e. number and type of RNAs to be investigated, is urgently required. By making it possible to synthesize and test a large number of different probe molecules on the surface of the reaction support in a very short time, with the processing equipment described it is possible to react very rapidly to changes of the molecular species to be investigated and adapt the analytical techniques.

### 6.26 Use of the Reaction Support for the Detection and Typing of Pathogens

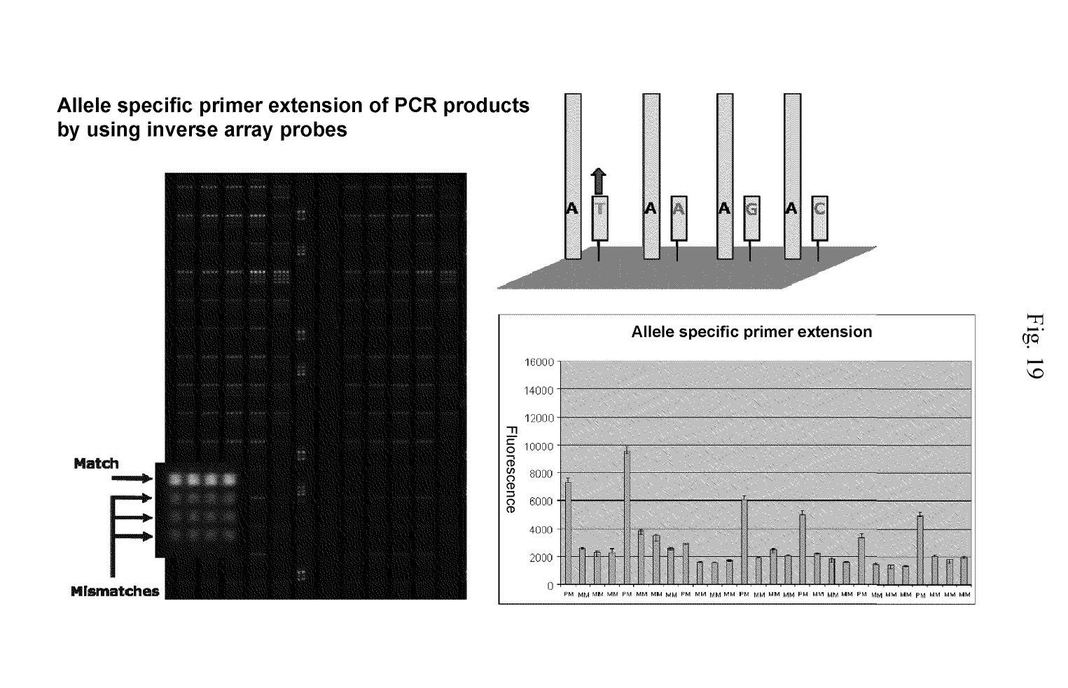

In another preferred embodiment the molecules synthesized on the surface of the reaction support are used for detecting and characterizing microorganisms and/or pathogens. In this, preferably particular nucleic acids that are characteristic of a particular pathogen or microorganism are bound selectively by the molecules synthesized on the surface of the reaction support. The nucleic acids used for this are preferably genes, mRNAs, genomic RNA or DNA or other RNAs of the pathogen. In addition, molecules synthesized on the surface of the reaction support are used for analyzing the identity of individual nucleotides in these nucleic acids. It is thus possible to elucidate mutations such as nucleotide substitutions, deletions or insertions. The methods used for this are based on the selective hybridization of molecules synthesized on the surface of the reaction support. Numerous methods for detecting such mutations are known by a person skilled in the art. As well as hybridization, which proceeds selectively for certain mutations, numerous other enzyme-based methods that are known by a person skilled in the art are used. The use of polymerases, nucleases and ligases is especially preferred. In all these cases the nucleic acids to be investigated are bound and/or used as substrate, and the efficiency of a reaction catalyzed by a particular enzyme changes when a mutation is present in the nucleic acid to be investigated. Alternatively, said efficiency can also change when, through binding of the nucleic acid to be investigated to the molecules synthesized on the surface of the reaction support, certain nucleotide pairs are produced, which for example differ in that they form pairs known by a person skilled in the art as Watson-Crick base pairs, or other pairs. Numerous techniques for said methods of detection are known by a person skilled in the art, such as APEX, allele-specific hybridization, allele-specific primer extension, allele-specific PCR, ASA, Taqman assays, molecular beacons, minisequencing, SSCP-PCR, mismatch cleavage, OLA, branch migration assay (BMA), pyrosequencing, dynamic allele specific hybridization (DASH), multiplex automated primer extension assay (MAPA) and many more. The methods described in 6.1, 6.5, 6.6, 6.9, 6.15, 6.16, 6.18, 6.19, 6.20 and 6.21 can also be used. FIG. 19 illustrates such an assay and shows data from successful identifications of individual nucleotide positions in a DNA sample. Use of these methods is especially preferred for differentiating pathogenic strains from one another and for making typing of pathogens possible. All species of pathogens occur in nature with various genotypes, which often differ from one another by just a few mutations, but have different properties, such as infection potential, resistance to certain medicinal products and much more. In an especially preferred embodiment, desired nucleic acids that are present in the sample mixture to be investigated, and originate from pathogens or in themselves represent pathogens, are amplified specifically. Owing to the ability to produce a large number of different sequences in the reaction support and use these as primers, a large number of amplification reactions individually adapted to the respective RNAs to be amplified can be carried out in parallel. The entire spectrum of amplification reactions that are known by a person skilled in the art is available. In particular, the methods described in 6.1, 6.5, 6.6, 6.9, 6.15, 6.16, 6.18, 6.19, 6.20 and 6.21 can be used. The nucleic acids to be investigated can then function either as template or as primer or both, and can be linked to universal sequences prior to amplification.

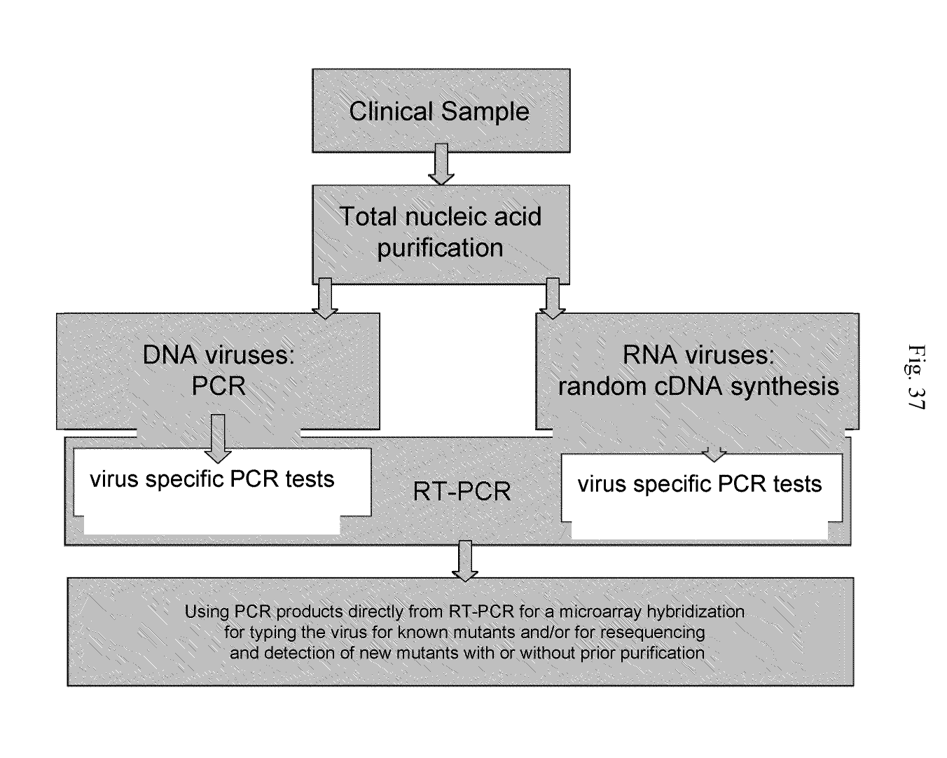

Molecular diagnostics is now employed in many areas, in order to detect microorganisms and viruses and quantify them in samples. In particular, quantitative real-time PCR is used for this. In current procedures, samples are for example investigated in individual PCRs or multiplex-PCRs for the content of a particular pathogen. If the test proves positive, it is followed by a large number of further investigations based on quantitative real-time PCR, in order to elucidate the precise genotype of the pathogenic species. This may for example be necessary for deciding about the nature of a chemotherapy or for setting up epidemiological investigations or for monitoring the populations of particular strains or for discovering the emergence of new strains. In an especially preferred embodiment, the products of the first real-time PCR, which was employed for detecting a pathogen, are used directly in the reaction support for elucidating the precise genotype of the pathogenic species. In that case the reaction support can be designed in such a way that both already known genotypes and new genotypes can be detected. FIG. 37 illustrates this preferred embodiment of the invention. Overall, use of the molecular-biological processing equipment described for the detection, quantification and genotyping of pathogens represents a considerable improvement over the prior art. As the genotypes of pathogens are highly variable, adaptation of analytical techniques to the conditions obtaining in each case is urgently required. By making it possible to synthesize and test a large number of different probe molecules on the surface of the reaction support in a very short time, with the processing equipment described it is possible to react very rapidly to changes of the samples to be investigated, such as pathogens, and adapt the analytical techniques.

### 6.27 Rapid Prototyping in the Molecular-Biological Processing Equipment Described

In another preferred embodiment, the processing equipment described is used for empirically testing probe molecules so that a large number of suitable probe molecules can be prepared very quickly for particular applications. It is known that the binding properties of probe molecules, e.g. oligonucleotides or derivatives or analogs of DNA or RNA, cannot be predicted very accurately. No bioinformatic methods exist for predicting the binding power and/or specificity of said probe molecules relative to desired sample molecules. There is therefore a need for empirical testing of probe molecules. After such an experiment, probes can then be selected in a “Rapid Prototyping Process” for particular quality criteria, which fulfill the desired conditions and are used in subsequent experiments. If we wish to investigate a sample for which there are still no known suitable probe molecules, or the latter are only suitable to a limited extent, it is then possible in model experiments to generate, test and/or optimize a number of probes very rapidly, which can then be used in later experiments for samples of this kind. In this way, the great flexibility of the molecular-biological processing equipment described offers a considerable advantage over the prior art. Most of the methods known by a person skilled in the art for the production of a large number of different probe molecules are either very slow or cannot do it at an economically acceptable price. In contrast, the great flexibility of the processing equipment described offers the possibility of generating, testing and/or optimizing a very large number of probe molecules in a very short time and at low cost, so as to react quickly to changes in the nature of the sample molecules to be investigated, i.e. the analytical technique can be adapted individually.

### 7 EXPERIMENTS

The invention is explained below, on the basis of the following experiments:

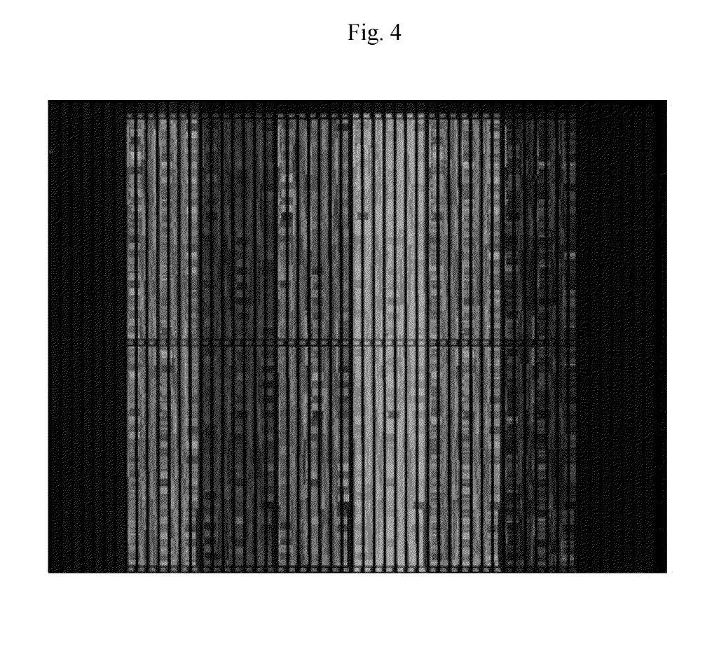

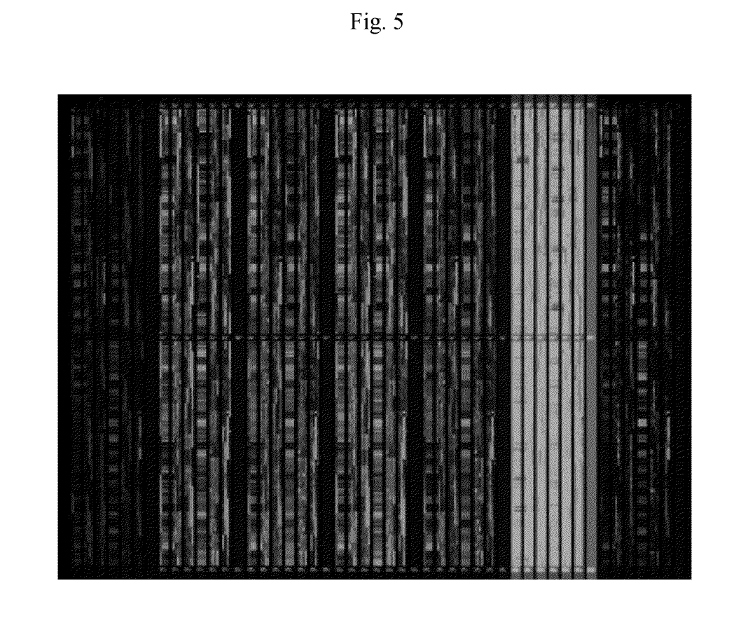

For the data shown in FIGS. 4 and 5, DNA probes with a length between 21-23 nucleotides were synthesized as in published methods in a microfluidic reaction support (Baum, M. et al., Nucleic Acids Research, 2003, 31, e151 and references cited there). Inverse synthetic chemistry was used, so that the probes were bound by the 5′ end to the surface of the reaction support and had a free 3′ end. The experiments were carried out using an external reaction unit, which made filling and temperature control of the reaction support possible.

The reaction support was filled with a mixture of 200 nM PCR product and 33 to 200 μM of each of the four dNTP (with 33% of the TTP replaced with Biotin-16-dUTP (Roche)) in the reaction buffer prepared by the manufacturer for the respective DNA polymerase, heated for 5 min at 80° C. and cooled to room temperature over the space of 20 min. The reaction support was thermostated at 37° C. for mesophilic DNA polymerases and at 72° C. for thermostable DNA polymerases, the mixture was removed and the reaction support was filled with the same mixture, which contained 1 μL enzyme per 15 μL. After a reaction time of 10 min, the reaction support was washed with 500 μL water. Within the molecular-biological processing equipment, the reaction support was incubated with a buffer solution containing streptavidin-phycoerythrin, washed and analyzed by fluorescence measurement.

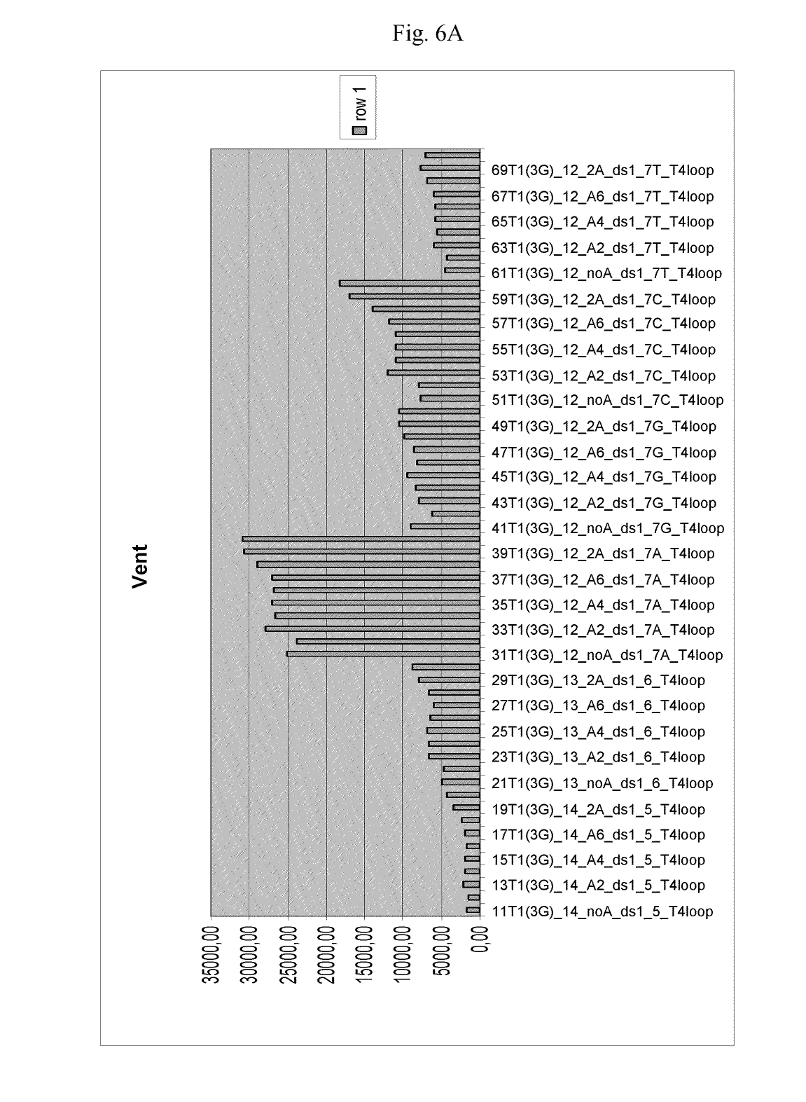

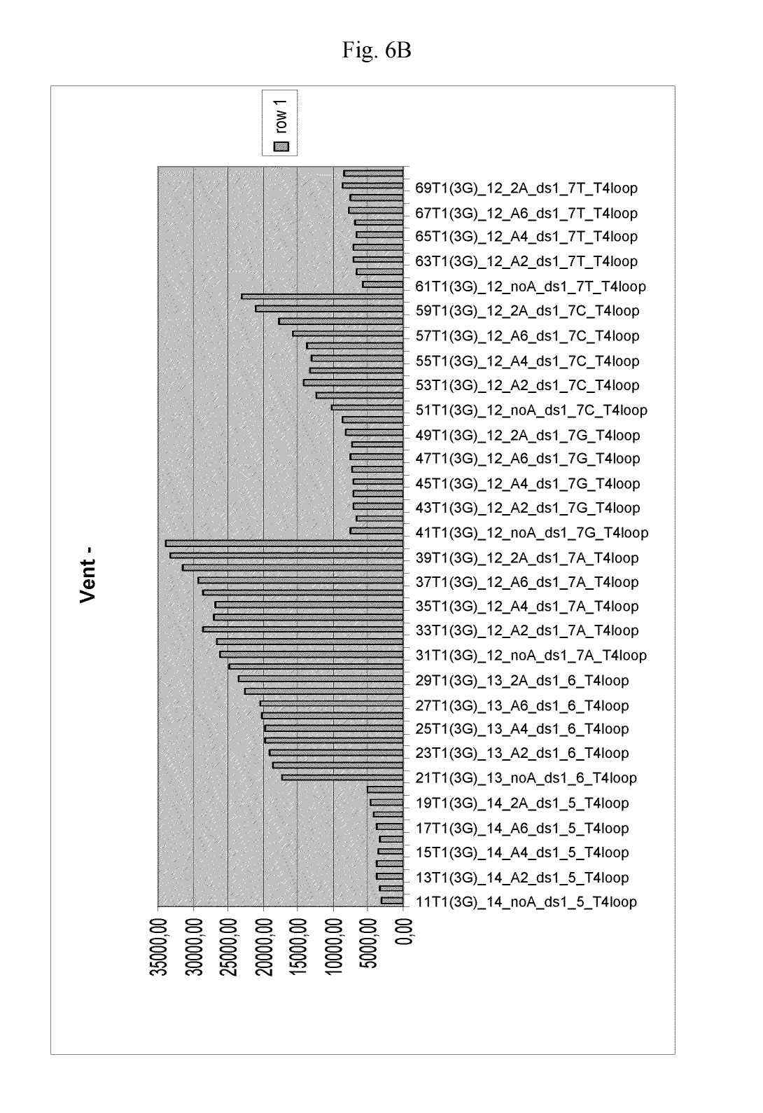

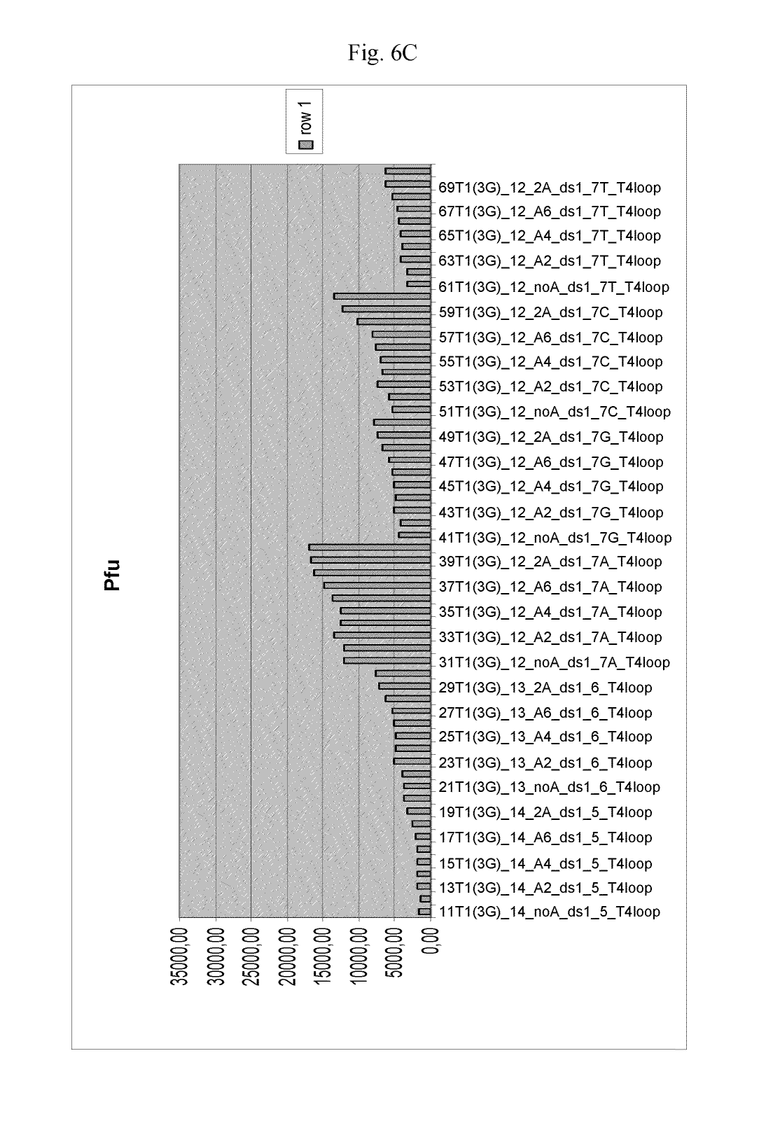

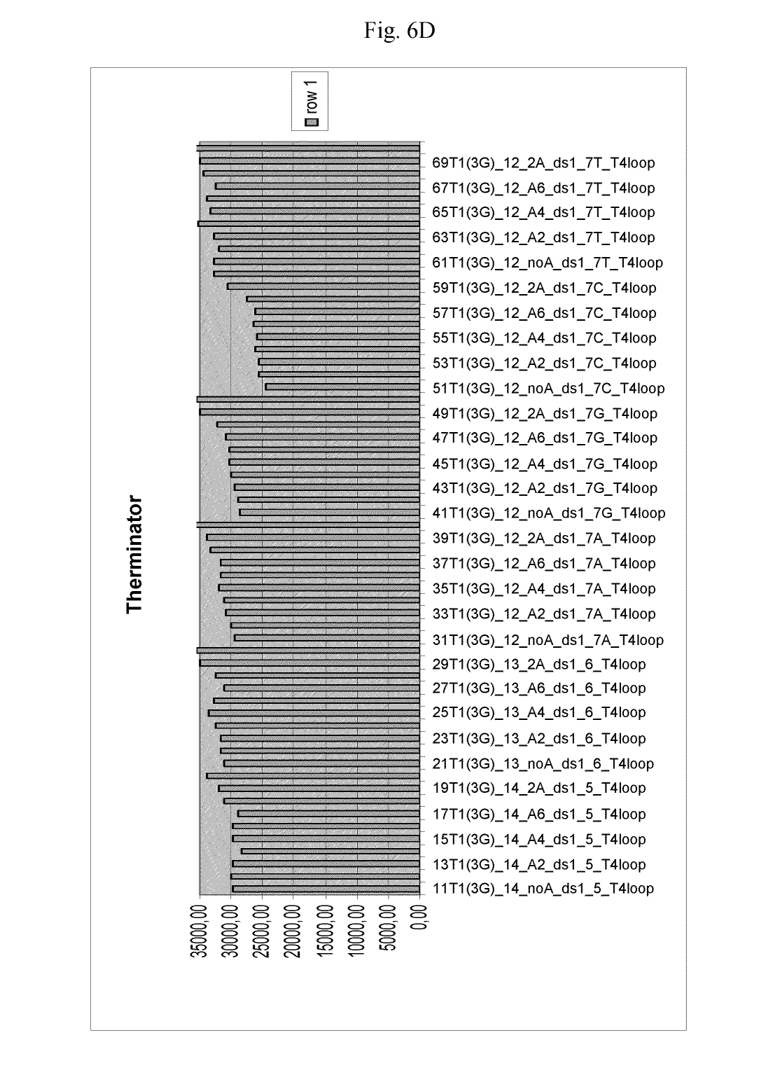

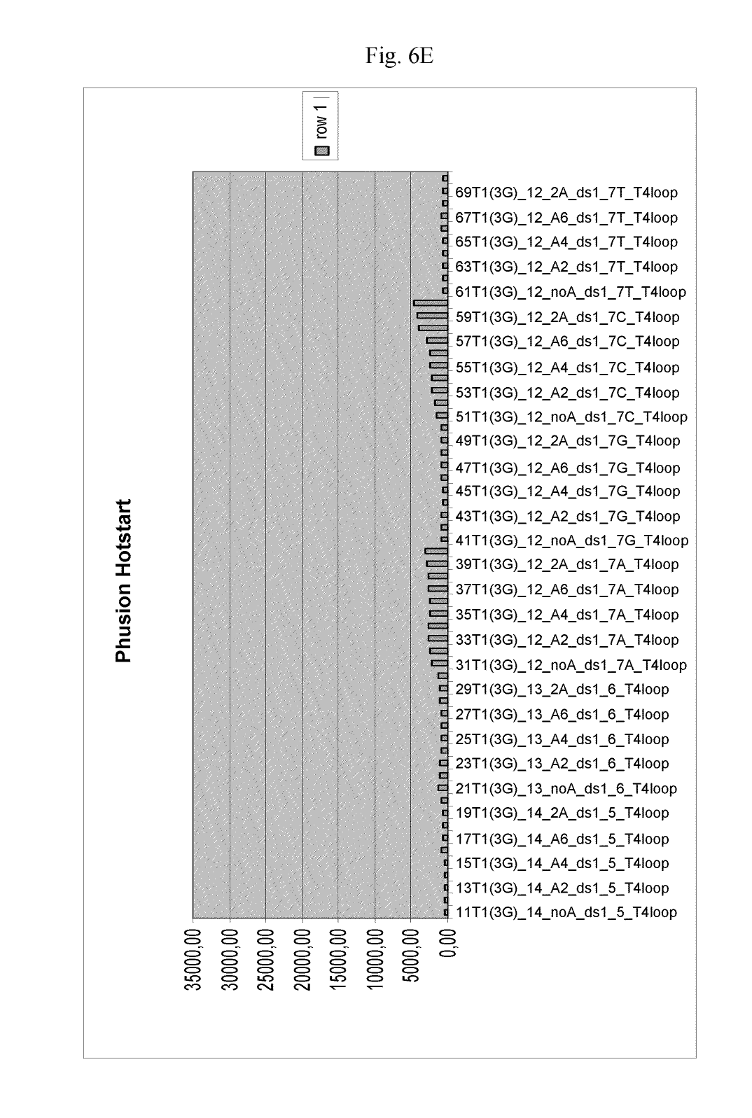

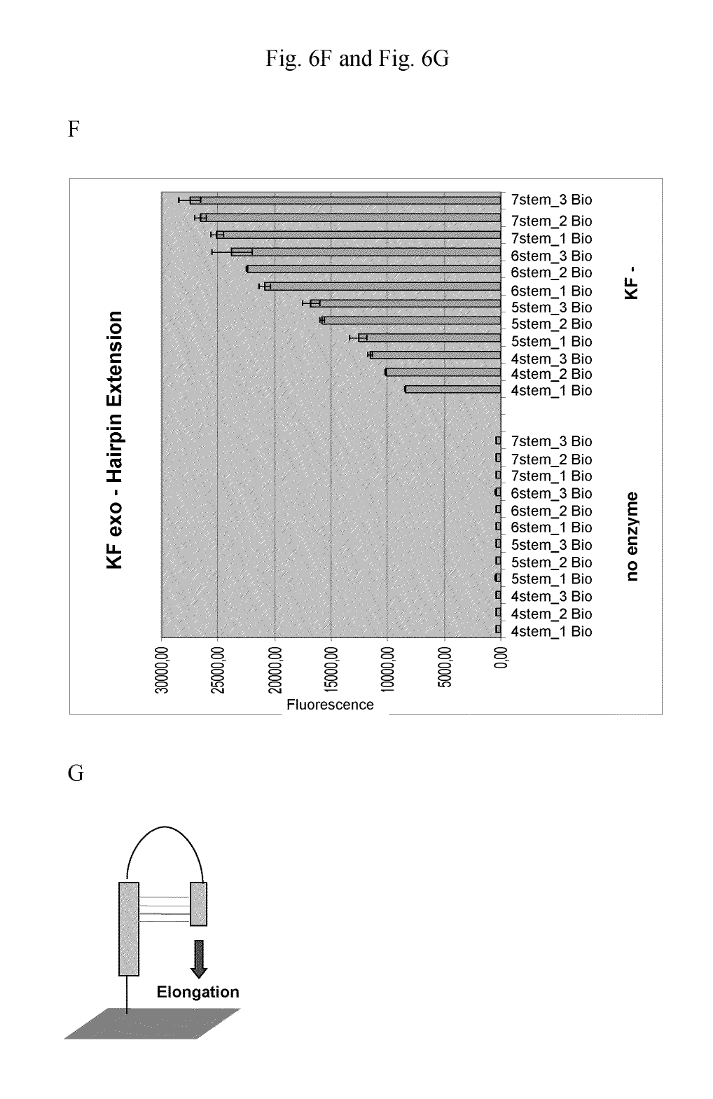

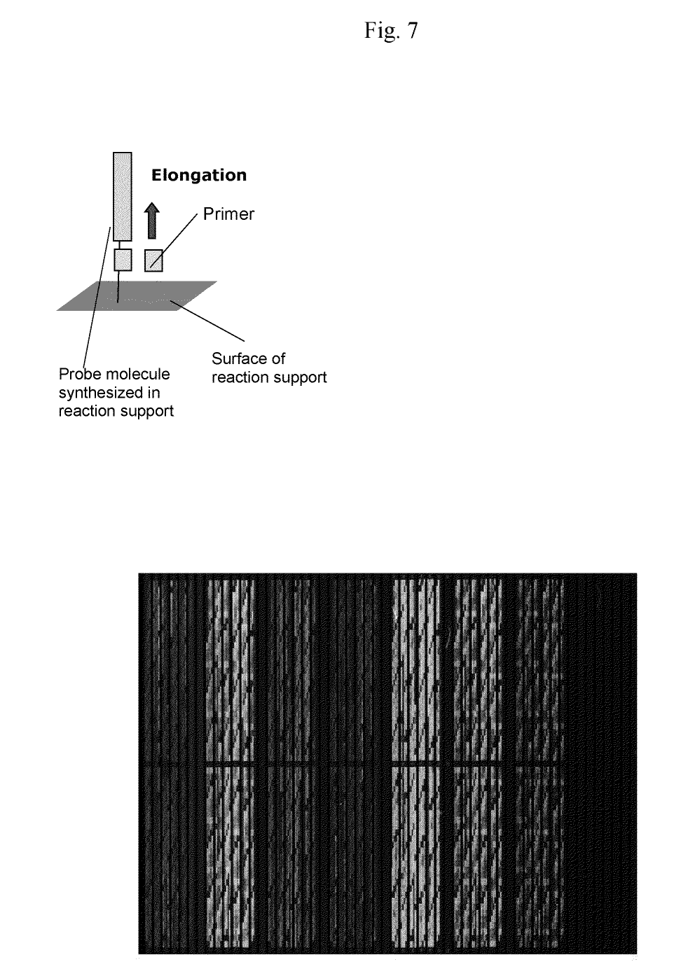

For the data shown in FIGS. 6 A-D, DNA probes were produced as above, which contain a self-complementary sequence and consequently form a hairpin structure, which possesses a paired 3′ end, which can be used by a DNA polymerase as primer. The probes had a length of 27 and 30 nucleotides, the length of the self-complementary region varied between 4-7 nucleotides, the loop between the self-complementary regions was in each case a TTTT sequence. The experiments were carried out as in the experiments described above relating to FIGS. 4 and 5.

For the data shown in FIGS. 7-9, probes were synthesized with a length between 30 and 50 nucleotides, which had various binding sites for primers, so that on hybridization, a primer-template complex forms with a single-strand region that can be copied by a DNA polymerase. The experiments were carried out as in the experiments described above relating to FIGS. 4 and 5, except that instead of the PCR product, corresponding primers were present at a concentration of 13 μM in the reaction mixture.

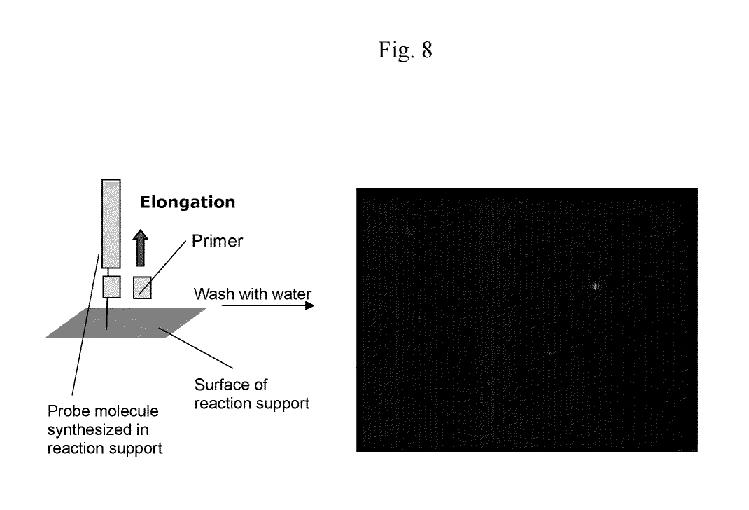

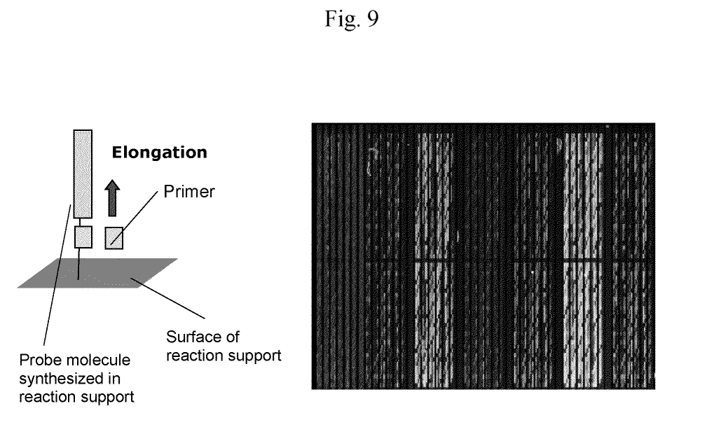

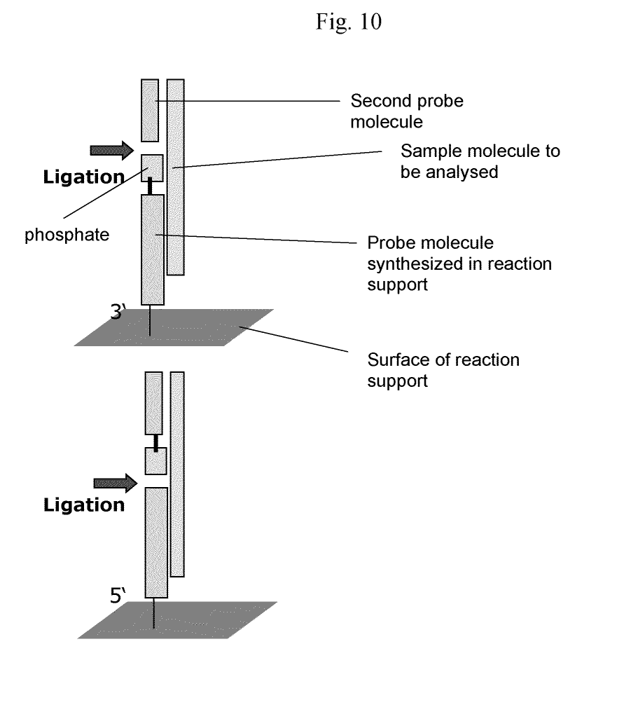

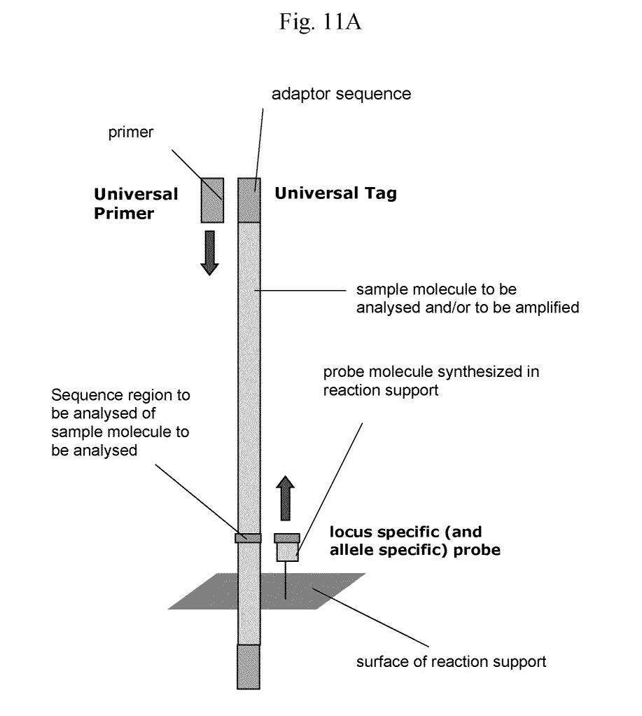

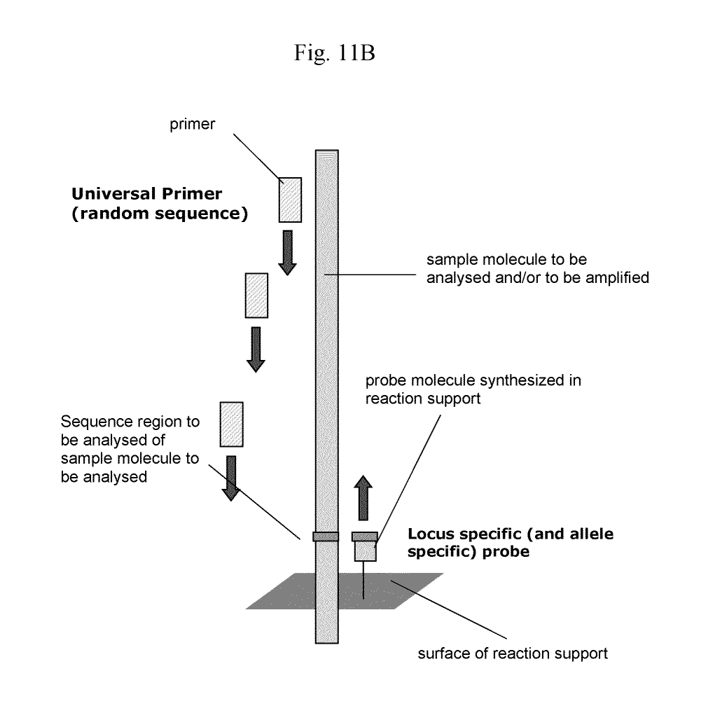

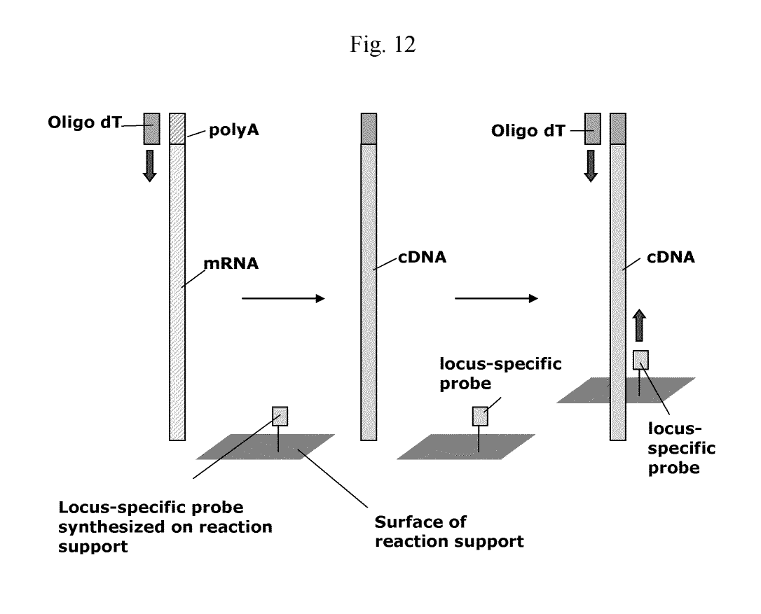

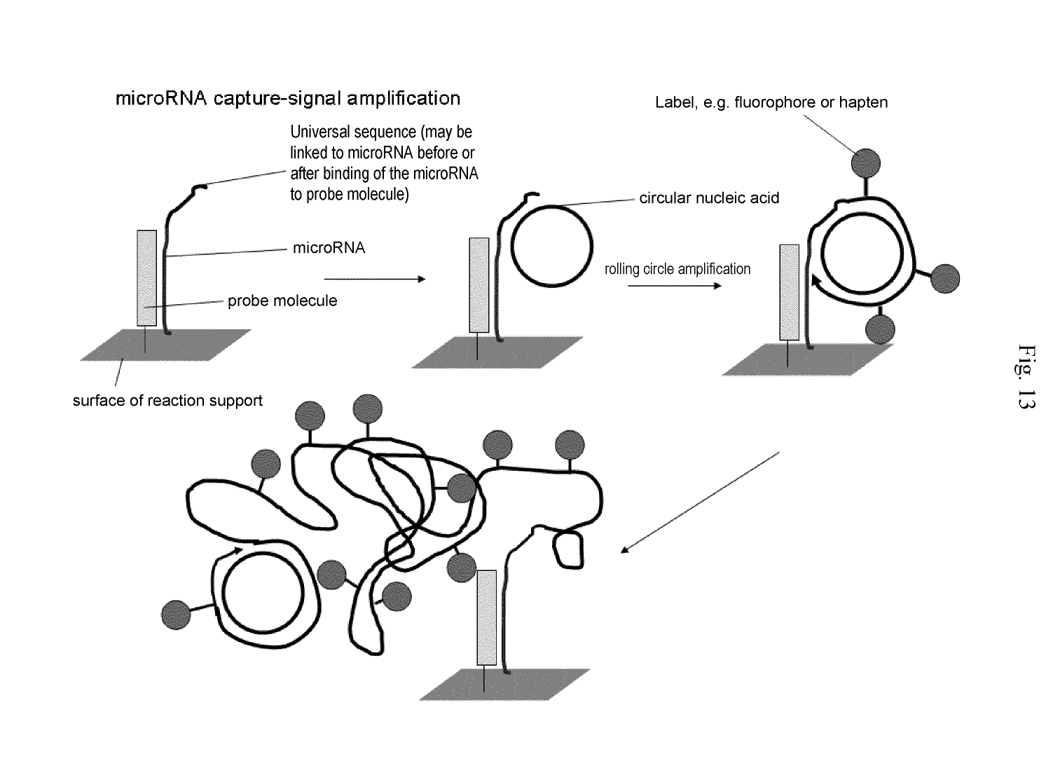

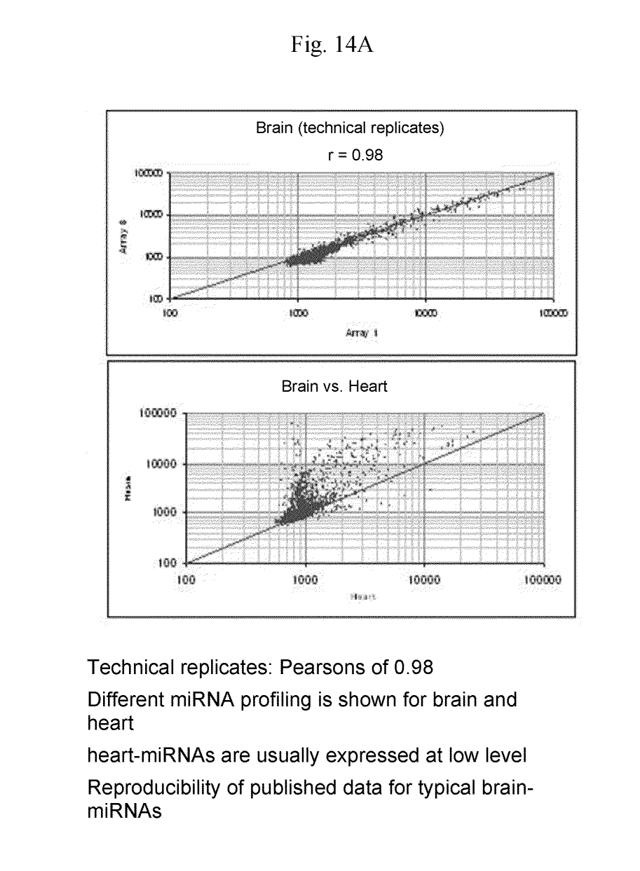

For the data shown in FIG. 8, after analysis of the reaction support by fluorescence measurement it was washed with water at 80° C. and analyzed again under the same conditions.

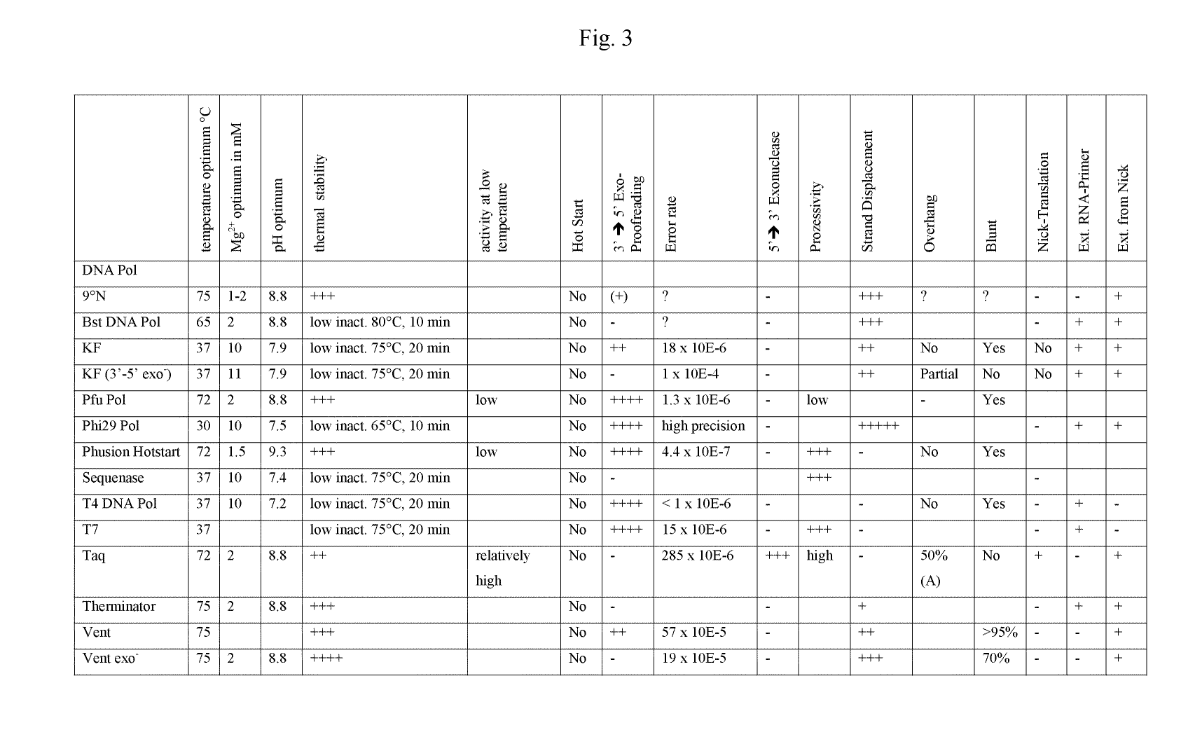

For the experiments shown in FIGS. 14-18, 1-5 μg of complete RNA was fractionated and purified by means of the flashPAGE kit that is known by a person skilled in the art; or fragmented and used further directly; or used directly; and labeled by means of the mirVana kit known by a person skilled in the art, according to the manufacturer's protocol. The purified, labeled RNA samples were introduced into the reaction support and were incubated at specified temperatures, with the buffer conditions as described in FIG. 15.

At the end of incubation, the solution was removed from the reaction support. The reaction support was incubated within the molecular-biological processing equipment with a buffer solution containing streptavidin-phycoerythrin, washed and analyzed by fluorescence measurement. Optionally, signal amplification was carried out as described in the description of FIGS. 16 and 17 (FIGS. 16 and 17).

For the data shown in FIGS. 19 and 23, DNA probes were synthesized with a length between 21-50 nucleotides as in the published methods in a microfluidic reaction support (Baum, M. et al., Nucleic Acids Research, 2003, 31, e151 and references cited there). For the data shown in FIG. 19, inverse synthetic chemistry was used, so that the probes were bound by the 5′ end to the surface of the reaction support and had a free 3′ end. For the data shown in FIG. 23, regular synthetic chemistry was used, so that the probes were bound by the 3′ end to the surface of the reaction support and had a free 5′ end. For the experiments, an external reaction unit was used, which made filling and temperature control of the reaction support possible.

FIG. 19: The reaction support was filled with a mixture of 200 nM PCR product and 200 μM of each of the four dNTP (with 33% of the TTP replaced with Biotin-16-dUTP (Roche)) in the reaction buffer for the respective DNA polymerase prepared by the manufacturer, heated for 5 min at 80° C. and cooled to room temperature in the space of 20 min. The reaction support was thermostated at 68° C., the mixture was removed and the reaction support was filled with the same mixture, which contained 1 μL of a Taq polymerase per 15 μL. After a reaction time of 15 min, the reaction support was washed with 500 μL. The reaction support was incubated within the molecular-biological processing equipment with a buffer solution containing streptavidin-phycoerythrin, washed and analyzed by fluorescence measurement.

FIG. 23: The reaction support was filled with a mixture of 13 μM of a primer and 33 μM of each of the four dNTP (with 33% of the TTP replaced with Biotin-16-dUTP (Roche)) in reaction buffer “2” of the company NEB, heated for 5 min at 80° C. and cooled to room temperature in the space of 20 min. The reaction support was thermostated at 37° C., the mixture was removed and the reaction support was filled with the same mixture, which contained 1 μL Klenow fragment (3′-5′-exo-). After a reaction time of 30 min the reaction support was washed with 500 μL water. The reaction support was incubated within the molecular-biological processing equipment with a buffer solution containing streptavidin-phycoerythrin, washed and analyzed by fluorescence measurement. The reaction support was washed with hot water at 80° C., and the same reaction was carried out once again, this time without Biotin-16-dUTP. The reaction support was washed with 25% ammonia solution, the eluate was dried, used as template in a PCR reaction, and investigated using gel electrophoresis.

For the data shown in FIGS. 20-22, DNA probes were synthesized as for the data shown in FIG. 19. For the experiments, an external reaction unit was used, which made filling and temperature control of the reaction support possible.

The reaction support was filled with a mixture of 5 μM primer, ˜214 μM PCR product, 200 μM of each of the four dNTP (with 33% of the TTP replaced with Biotin-16-dUTP (Roche)) and 0.1 U/μL Taq DNA polymerase (NEB) in 20 mM Tris-HCl pH=8.8, 10 mM KCl, 10 mM ammonium sulfate and 2 mM magnesium sulfate. Next the reaction support was submitted to a temperature profile as shown in FIG. 20 (s=seconds). At the end of the temperature program the solution was removed from the reaction support. The reaction support was incubated within the molecular-biological processing equipment with a buffer solution containing streptavidin-phycoerythrin, washed and analyzed by fluorescence measurement.

1. A molecular-biological processing equipment comprising
(a) a device configured to undertake an in-situ synthesis of arrays of receptors,
(b) one or more elements configured to execute one or more fluidic steps,
(c) a detection unit configured to detect an optical or electrical signal,
(d) a programmable unit configured to control the in-situ synthesis, and
(e) a programmable unit configured to control the fluidic steps, and to detect, store and manage measurement data.

2. The molecular-biological processing equipment as claimed in claim 1, wherein the processing equipment has one or more flow cells and in that the one or more fluidic steps in the one or more flow cells takes 1 min or less.

3. The molecular-biological processing equipment as claimed in claim 1, wherein the processing equipment has one or more flow cells and in that a fluid volume in the one or more flow cells is equal to 40% or less of a volume of a feed line connected to a fluid reservoir.

4. The molecular-biological processing equipment as claimed in claim 1, wherein the processing equipment has one or more flow cells and that in the execution of the fluidic steps at least 2 different reagents are introduced into the one or more flow cells.

5. The molecular-biological processing equipment as claimed in claim 4, wherein in the execution of the fluidic steps the at least two different reagents are introduced in 10 min or less into the one or more flow cells.

6. The molecular-biological processing equipment as claimed in claim 1, wherein the receptors comprise oligopeptides, polypeptides, oligonucleotides, polynucleotides or combinations thereof.

7. The molecular-biological processing equipment as claimed in claim 1, additionally comprising a light source matrix that optionally is programmable.

8. The molecular-biological processing equipment as claimed in claim 1, wherein the device comprises a support having several predetermined positions that are configured to immobilize the receptors.

9. The molecular-biological processing equipment as claimed in claim 8, wherein particular predetermined positions are configured to immobilize different receptors.

10. The molecular-biological processing equipment as claimed in claim 8, wherein the device comprises means for feeding fluids into the support and for withdrawing fluids from the support.

11. The molecular-biological processing equipment as claimed in claim 8, wherein the detection unit comprises a detection matrix comprising several detectors that are assigned to the predetermined positions on the support.

12. The molecular-biological processing equipment as claimed in claim 11, wherein the support is located between the light source matrix and the detection matrix.

13. The molecular-biological processing equipment as claimed in claim 8, wherein the receptors comprise nucleic acids, nucleic acid analogs or combinations thereof, and that in one or more of the predetermined positions the receptors, in the absence of an analyte that can specifically bind thereto, at least partially form a secondary structure.

14. The molecular-biological processing equipment as claimed in claim 13, wherein the secondary structure comprises a hairpin structure.

15. The molecular-biological processing equipment as claimed in claim 8, wherein the receptors comprise nucleic acids, nucleic acid analogs or combinations thereof, and in that the receptors comprise several different types of receptor building blocks.

16. A method of analysis of a nucleic acid sequence of a nucleic acid analyte comprising:
(a) in-situ synthesis of at least one oligonucleotide probe in at least one synthesis region in a flow cell;
(b) addition of at least one single-stranded or double-stranded nucleic acid analyte to the flow cell; and
(c) ligation or sequence-specific hybridization of the nucleic acid analyte to the oligonucleotide probe;
wherein at least one template-dependent nucleic acid synthesis step is accompanied by a change in an optical or electrical signal.

17. The method as claimed in claim 16, wherein an internal volume of the flow cell is equal to 40% or less of a volume of a feed line connected to a fluid reservoir.

18. The method as claimed in claim 16, wherein the flow cell is configured such that a fluidic step takes 1 min or less.

19. The method as claimed in claim 16, additionally comprising at least one step of nucleic acid amplification before the at least one template-dependent nucleic acid synthesis step.

20. The method as claimed in claim 16, wherein the nucleic acid analyte is selected from the group comprising:
a microRNA,
a cDNA corresponding to a microRNA,
a nucleic acid with pathogenic action,
a nucleic acid obtained from a pathogen,
a genomic DNA,
a cDNA and combinations thereof.

21. A method of amplification of a target nucleic acid comprising:
(a) in-situ synthesis of at least one oligonucleotide probe in at least one synthesis region in a flow cell;
(b) addition of at least one single-stranded or double-stranded nucleic acid analyte to the flow cells;
(c) ligation or sequence-specific hybridization of the nucleic acid analyte to the oligonucleotide probe; and
(d) at least one cycle of nucleic acid amplification.

22. The method as claimed in claim 21, wherein the nucleic acid analyte is selected from the group comprising:
a microRNA,
a cDNA corresponding to a microRNA,
a nucleic acid with pathogenic action,
a nucleic acid obtained from a pathogen,
a genomic DNA,
a cDNA and combinations thereof.

23. A method of amplification of a target nucleic acid comprising:
(a) in-situ synthesis of at least one oligonucleotide probe in at least one synthesis region in a flow cell; wherein the oligonucleotide probe has intramolecular hybridization regions; wherein one of the intramolecular hybridization regions is positioned at the 3′ end of the oligonucleotide probe; and wherein a recognition sequence for a nicking endonuclease
(i) is present in the hybridization region at the 3′ end of the oligonucleotide probe, or
(ii) can be generated by a sequence-dependent extension of the hybridization region at the 3′ end of the oligonucleotide probe, or
(iii) is partially present in the hybridization region at the 3′ end of the oligonucleotide probe and can be completed by a sequence-dependent extension of the hybridization region at the 3′ end of the oligonucleotide probe;
(b) sequence-specific hybridization of the intramolecular hybridization regions of the oligonucleotide probe to one another;
(c) addition of a DNA polymerase;
(d) sequence-dependent synthesis of a complementary DNA strand by the DNA polymerase starting from the 3′ end of the oligonucleotide probe;
(e) addition of a nicking endonuclease;
(f) production of a recognition-sequence-specific single-strand break by the nicking endonuclease;
(g) sequence-dependent synthesis of a new complementary DNA strand by the DNA polymerase starting from the single-strand break produced in (f) with displacement of the previously synthesized complementary DNA strand; and
(h) optionally single or multiple repetition of steps (f) and (g);
wherein step (c) can take place before, during or after step (b); and wherein step (e) can take place before, during or after one of steps (b), (c) or (d).

24. A method of amplification of a target nucleic acid comprising:
(a) in-situ synthesis of at least one oligonucleotide probe in at least one synthesis region in a flow cell;
(b) addition of a primer molecule; wherein the primer is designed so that, at least at its 3′ end, it has a region that is complementary to the oligonucleotide probe; and wherein a recognition sequence for a nicking endonuclease
(i) is present in the region complementary to the oligonucleotide probe of the primer, or
(ii) can be generated by a sequence-dependent extension of the region complementary to the oligonucleotide probe, or
(iii) is partially present in the region complementary to the oligonucleotide probe of the primer and can be completed by a sequence-dependent extension of the region complementary to the oligonucleotide probe of the primer;
(c) sequence-specific hybridization of the primer molecule to the oligonucleotide probe;
(d) addition of a DNA polymerase;
(e) sequence-dependent synthesis of a complementary DNA strand by the DNA polymerase starting from the 3′ end of the primer molecule;
(f) addition of a nicking endonuclease;
(g) production of a recognition-sequence-specific single-strand break by the nicking endonuclease;
(h) sequence-dependent synthesis of a new complementary DNA strand by the DNA polymerase starting from the single-strand break produced in (g) with displacement of the previously synthesized complementary DNA strand; and
(i) optionally single or multiple repetition of steps (g) and (h);
wherein step (d) can take place before, during or after one of the steps (b) or (c); and wherein step (f) can take place before, during or after one of steps (b), (c), (d) or (e).

25. A method of amplification of a target nucleic acid comprising:
(a) in-situ synthesis of a plurality of at least one first oligonucleotide probe in at least one synthesis region in a flow cell;
(b) in-situ synthesis of a plurality of at least one second oligonucleotide probe in at least one synthesis region in a flow cell; wherein the distance between any two oligonucleotide probes in each case is selected so that they cannot bind to one another; wherein in each case appropriate first and second oligonucleotide probes are synthesized in the same synthesis region.
(c) addition of at least one single-stranded or double-stranded nucleic acid analyte to the flow cell;
(d) ligation or sequence-specific hybridization of the nucleic acid analyte to a first oligonucleotide probe;
(e) addition of a DNA polymerase;
(f) sequence-dependent synthesis of a complementary DNA strand by the DNA polymerase starting from the 3′ end of the first oligonucleotide probe;
(g) ligation or sequence-specific hybridization of the DNA strand newly synthesized in (f) to a second oligonucleotide probe;
(h) sequence-dependent synthesis of a complementary DNA strand by the DNA polymerase starting from the 3′ end of the second oligonucleotide probe;
(i) optionally ligation or sequence-specific hybridization of the DNA strand newly synthesized in (h) to a first oligonucleotide probe; and
(j) optionally single or multiple repetition of steps (f) to (i);
wherein step (e) can take place before, during or after one of steps (b), (c) or (d).

26. The method as claimed in claim 25, further comprising:
(A) a stringent washing step after step (d); or
(B) a stringent washing step after step (f).

27. The method as claimed in claim 21, wherein the amount of newly synthesized nucleic acids is determined in real time.

28. A method of production of a support for the determination of nucleic acid analytes by hybridization, comprising:
(a) provision of a supporting material and
(b) stepwise construction of an array of several different receptors selected from nucleic acids and nucleic acid analogs on the support by spatially-specific and/or time-specific immobilization of receptor building blocks at respective predetermined positions on or in the supporting material,
wherein for the synthesis of the receptors several different sets of synthetic building blocks are used, in order to obtain asymmetric receptors.

29. A method of production of a support for the determination of nucleic acid analytes by hybridization, comprising:
(a) provision of a supporting material and
(b) stepwise construction of an array of several different receptors selected from nucleic acids and nucleic acid analogs on the support by spatially specific and/or time-specific immobilization of receptor building blocks at respective predetermined positions on or in the supporting material,
wherein in one or more of the predetermined positions the nucleotide sequences of the receptors are selected in such a way that the receptors, in the absence of an analyte that can bind specifically to them, are at least partially in the form of a secondary structure.

30. A method of determination of analytes, comprising:
(a) provision of a support with several predetermined regions, on which in each case different receptors, selected from nucleic acids and nucleic acid analogs, are immobilized, wherein in one or more of the predetermined regions the receptors consist of several different types of receptor building blocks,
(b) contacting the support with a sample containing analytes and
(c) determining the analytes from their binding to the receptors immobilized on the support, wherein the binding of an analyte to a receptor specifically bindable thereto leads to a detectable change in signal.

31. A method of determination of analytes, comprising:
(a) provision of a support with several predetermined regions, on which in each case different receptors, selected from nucleic acids and nucleic acid analogs, are immobilized, wherein in one or more of the predetermined regions the receptors, in the absence of an analyte that can bind specifically to them, are at least partially in the form of a secondary structure,
(b) contacting the support with a sample containing analytes and
(c) determining the analytes from their binding to the receptors immobilized on the support, wherein the binding of an analyte to a receptor specifically bindable thereto comprises the detection of the opening of the secondary structure that is present in the absence of the analyte.

32. The method as claimed in claim 30, wherein the analyte is selected from the group comprising:
a microRNA,
a cDNA corresponding to a microRNA,
a nucleic acid with pathogenic action,
a nucleic acid obtained from a pathogen,
a genomic DNA,
a cDNA and combinations thereof.

33. A method of determination of analytes, comprising:
(a) provision of a support with several predetermined regions, on which in each case different receptors, selected from nucleic acids and nucleic acid analogs, are immobilized; wherein each individual receptor comprises at least one hybridization region, to which an analyte can hybridize specifically;
(b) contacting the support with a sample containing analytes;
(c) execution of a primer extension reaction; wherein the analyte functions as primer; wherein building blocks carrying one or more signal-emitting groups and/or one or more haptens, are incorporated in the primer extension reaction; and
(d) determination of the analyte from the incorporation of building blocks containing signal groups or haptens.

34. The method as claimed in claim 33, further comprising:
incubation with a ssDNA-specific nuclease between step (b) and (c).

35. A method of determination of analytes, comprising:
(a) provision of a support with several predetermined regions, on which in each case different receptors, selected from nucleic acids and nucleic acid analogs, are immobilized; wherein each individual receptor comprises at least one hybridization region, to which an analyte can hybridize specifically;
(b) contacting the support with a sample containing analytes; wherein the analytes in the sample were linked, before, during or after the contacting, to one or more signal-emitting groups and/or to one or more haptens;
(c) determination of the analytes by detecting the signal-emitting group(s) or the hapten or haptens in the analyte.

36. The method as claimed in claim 33, wherein the analyte is a nucleic acid, selected from the group comprising:
a microRNA,
a cDNA corresponding to a microRNA,
a nucleic acid with pathogenic action,
a nucleic acid obtained from a pathogen,
a genomic DNA,
a cDNA and combinations thereof.

37. A method of amplification of analytes, comprising:
(a) provision of a support with several predetermined regions, on which in each case different receptors, selected from nucleic acids and nucleic acid analogs, are immobilized; wherein each individual receptor has, at its 3′ end, a hybridization region to which an analyte can hybridize specifically;
(b) contacting the support with a sample containing analytes; and
(c) execution of a primer extension reaction; wherein the various receptors function as primers, so that a double-stranded nucleic acid comprising an analyte and an extended receptor, is obtained.

38. The method as claimed in claim 37, further comprising following step (c):
(d) thermal denaturation of the double-stranded nucleic acid obtained in step (c);
(e) setting of reaction conditions that permit hybridization of analyte and nonextended receptors;
(f) execution of a primer extension reaction, with the various nonextended receptors functioning as primers; and
(g) optionally repetition of steps (d) to (f).

39. The method as claimed in claim 37, wherein in the primer extension reaction (c) according to claim 37 bears one or more signal-emitting groups and/or one or more haptens.

40. The method as claimed in claim 39, further comprising during one of steps (c) to (g) or after one of steps (c) to (g):
determination of the analyte from the incorporation of the signal-group-containing and/or hapten-containing building blocks.

41. The method as claimed in claim 37; wherein the analyte is an RNA; wherein the various receptors additionally have a region with a primer sequence 1, in the 5′ position to the hybridization region, and further comprising following step (c):
(d) ligation of a nucleic acid cassette, which has a region with a primer sequence 2, to the double-stranded nucleic acid obtained in step (c);
(e) execution of a two-strand synthesis;
(f) execution of at least one cycle of an amplification reaction with addition of a primer with primer sequence 1 and a primer with primer sequence 2.

42. The method as claimed in claim 41, wherein in step (e) and/or step (f) building blocks are incorporated that bear one or more signal-emitting groups and/or one or more haptens.

43. The method as claimed in claim 42, further comprising during one of steps (e) to (f) or after one of steps (e) to (f):
determination of the analyte from the incorporation of the signal-group-containing and/or hapten-containing building blocks.

44. The method as claimed in claim 37, wherein the analyte is a nucleic acid, selected from the group comprising:
a microRNA,
a cDNA corresponding to a microRNA,
a nucleic acid with pathogenic action,
a nucleic acid obtained from a pathogen,
a genomic DNA,
a cDNA and combinations thereof.

45. A method of production of a support for nucleic acid analysis and/or synthesis, comprising:
(a) providing a supporting material and
(b) stepwise constructing an array of several different receptors comprising nucleic acids or nucleic acid analogs on the support by spatially-specific and/or time-specific immobilization of receptor building blocks at respective predetermined positions on or in the supporting material, wherein in at least one synthesis region, at least 2 different receptors are synthesized by an orthogonal chemical method.

46. The method as claimed in claim 16, wherein the method comprises using the molecular-biological processing equipment as claimed in claim 1.

47. A reagent kit, comprising a supporting material and at least two different sets of building blocks configured to synthesize receptors on the supporting material.

48. The reagent kit as claimed in claim 47 further comprising reaction liquids.

49. The reagent kit as claimed in claim 47, wherein the building blocks comprise deoxyribonucleotides, ribonucleotides, N3′-P5′-phosphoroamidate (NP) building blocks, locked nucleic acid (LNA) building blocks, morpholinophosphorodiamidate (MF) building blocks, 2′-O-methoxyethyl (MOE) building blocks, 2′-fluoro-arabino-nucleic acid (FANA) building blocks, phosphorothioate (PS) building blocks, 2′-O-methyl (OMe) building blocks, peptide nucleic acid (PNA) building blocks, or combinations thereof.

50. A method comprising applying the molecular-biological processing equipment as claimed in claim 1 for an application comprising:
detection and/or for the isolation of nucleic acids;
sequencing;
point mutation analysis;
analysis of genomes, genome variations, genome instabilities and/or chromosomes;
typing of pathogens;
genotyping;
gene-expression or transcriptome analysis;
analysis of cDNA libraries;
production of substrate-bound cDNA libraries or cRNA libraries;
production of arrays for the production of synthetic nucleic acids; nucleic acid double strands and/or synthetic genes;
production of arrays of primers, ultra-longmers, probes for homogeneous assays, molecular beacons and/or hairpin probes;
production of arrays for the production, optimization and/or development of antisense molecules;
further processing of the analytes or target molecules for logically downstream analysis on the microarray, in a sequencing process, in an amplification process or for analysis in gel electrophoresis;
the production of processed RNA libraries for subsequent steps, selected from: translation in vitro or in vivo or modulation of gene expression by iRNA or RNAi;
production of sequences that are then cloned by vectors or in plasmids or directly; and/or
ligation of nucleic acids in vectors or plasmids.

51. The method as claimed in claim 50, wherein the sequencing is a sequencing-by-synthesis method.

52. The method as claimed in claim 50, wherein the point mutation analysis is an SNP-analysis or a detection of new SNPs.

53. The method as claimed in claim 50, wherein the arrays produced on primers can be used for primer-extension methods, strand displacement amplification, polymerase chain reaction (PCR), site directed mutagenesis or rolling circle amplification.

54. The method as claimed in claim 50, wherein the logically downstream analysis on the microarray is an amplification method, selected from strand displacement amplification, polymerase chain reaction and rolling circle amplification.

55. The method as claimed in claim 50, wherein the nucleic acid to be detected and/or to be isolated is selected from the group comprising:
a microRNA,
a cDNA corresponding to a microRNA,
a nucleic acid with pathogenic action,
a nucleic acid obtained from a pathogen,
a genomic DNA,
a cDNA and combinations thereof.
